(19) United States
(12) Patent Application Publication
(10) Pub. No.: US 2020/0230221 A1
(43) Pub. Date: Jul. 23, 2020

(54) COMPOSITIONS FOR CHIMERIC ANTIGEN RECEPTOR T CELL THERAPY AND USES THEREOF

(71) Applicant: Massachusetts Institute of Technology, Cambridge, MA (US)

(72) Inventors: Darrell J. IRVINE, Arlington, MA (US); Leyuan MA, Brookline, MA (US)

(21) Appl. No.: 16/644,893

(22) PCT Filed: Sep. 19, 2018
(86) PCT No.: PCT/US2018/051764

§ 371 Date: Mar. 5, 2020

Related U.S. Application Data
(60) Provisional application No. 62/560,588, filed on Sep. 19, 2017.

(51) Int. Cl. A61K 39/00; A61K 47/54; C07K 14/47; A61K 39/39; A61K 35/17; A61K 9/00

(52) U.S. Cl. CPC A61K 39/0011; A61K 47/543; A61K 9/0029; A61K 39/39; A61K 35/17; C07K 14/4748

(57) ABSTRACT

The disclosure describes amphiphilic ligand conjugates comprising a chimeric antigen receptor (CAR) ligand, a lipid (diacyl lipid), a linker (hydrophilic polymers, hydrophilic amino acids, polysaccharides), compositions and methods of using the constructs are claimed, for example, to stimulate proliferation of CAR expressing cells.

### RELATED INFORMATION PARAGRAPH

This application claims the benefit of the priority date of U.S. Provisional Application No. 62/560,588, filed on Sep. 19, 2017, the content of which is hereby incorporated by reference in its entirety.

### BACKGROUND

Dramatic advances are happening in the clinical treatment of cancer using immunotherapy. One of the most powerful treatments developed to date is adoptive cell therapy with chimeric antigen receptor T cells (CAR T cells or CAR-T). CAR-T are autologous lymphocytes from a patient transduced with a synthetic antigen receptor, formed by fusing an antigen-binding domain to the CD3 signaling chain from the T cell receptor complex, and a costimulatory domain from one of multiple well known co-receptors that provide supporting signals during T cell activation. CAR-T cells have shown dramatic complete responses in hematologic malignancies, and the FDA recently approved a CAR-T therapy for treatment of B cell leukemia.

However, CAR-T cells currently are simply infused into patients, and receive no additional stimulation except through encounter of tumor cells in vivo, which lack many of the key signaling cues normally provided to T cells to promote their full effector function. In addition, CAR-T cells fail to functionally persist in some patients, and show generally poor responses in solid tumors. Accordingly, there exists a need for agents that improve CAR-T cell therapy.

### SUMMARY OF DISCLOSURE

The present disclosure is based, at least in part, on the discovery that chimeric antigen receptor (CAR) ligands are delivered efficiently to lymph nodes by use of an amphiphile conjugate which binds human serum albumin and partitions into membranes of resident antigen presenting cells (APCs), thereby co-displaying a CAR-T cell ligand on the cell surface together with native cytokine/receptor co-stimulation signals. Without being bound by theory, it is believed that these dual properties of amphiphile conjugates (i.e., lymph node targeting and membrane insertion) combine to enable a booster vaccine for CAR-T cells, which expands CAR-T cells efficiently in vivo, increases their functionality, and enhances anti-tumor activity.

It has been demonstrated that an amphiphilic ligand conjugate comprising either a tag or a tumor-associated antigen activated and induced proliferation of T cells expressing a CAR comprising a tag or tumor-associated antigen binding domain, or both. Notably, such amphiphilic ligand conjugates retained this activity in vivo, thus allowing for expansion and activation of CAR-T cells after administration to a subject. Further, administration of amphiphilic ligand conjugates of the disclosure also resulted in significantly increased CAR-T infiltration into tumors, and tumor-infiltrating CAR-T cells exhibited enhanced reactivity against tumor cells despite surface expression of checkpoint inhibitors PD1 and TIM3. Treatment with amphiphilic ligand conjugates of the disclosure with CAR-T cell therapy significantly delayed tumor growth and prolonged survival.

The present disclosure is also based, at least in part, on the discovery that the amphiphilic ligand conjugates described herein overcome the poor responses of CAR-T cells shown in solid tumors. As demonstrated herein, administration of CAR-T cells expressing a tumor-associated antigen were capable of delaying tumor growth of solid tumors and increasing the survival of tumor-bearing mice when administered in combination with an amphiphilic ligand conjugate, compared to control and CAR-T cells alone.

Further, the disclosure is based, at least in part, on the discovery that the enhanced efficacy of CAR-T cell therapy in combination an amphiphilic ligand conjugate of the disclosure is maintained in lymphreplete conditions. Current CAR-T cell therapy requires lymphodepletion, which is associated with serious toxicities. As shown herein, CAR-T cell therapy in combination with an amphiphilic ligand conjugate of the disclosure resulted in delayed tumor growth and increased survival of lymphreplete tumor-bearing mice. The delayed tumor growth and increased survival was comparable to lymphodepleted mice that received the same therapeutic regimen. Without wishing to be bound by theory, these results indicate administration of an amphiphilic ligand conjugate of the disclosure may negate the need for lymphodepletion prior to CAR-T cell therapy, thereby mitigating toxicity in a subject.

Accordingly, in one aspect the present disclosure provides an amphiphilic ligand conjugate comprising a chimeric antigen receptor (CAR) ligand, and a lipid operably linked to the CAR ligand. In some aspects, the lipid inserts in a cell membrane under physiological conditions. In some aspects, the lipid binds to albumin under physiological conditions. In some aspects, the lipid inserts in a cell membrane under physiological conditions and binds albumin under physiological conditions. In some aspects, the amphiphilic ligand conjugate comprises a lipid which traffics to lymph nodes and inserts into cell membranes of resident antigen presenting cells (APCs), thereby co-displaying a CAR-T cell ligand on the cell surface together with native cytokine/receptor co-stimulation signals.

In any of the foregoing or related aspects, the amphiphilic ligand conjugate of the disclosure comprises a diacyl lipid. In some aspects, the diacyl lipid comprises acyl chains comprising 12-30 hydrocarbon units. In some aspects, the diacyl lipid comprises acyl chains comprising 14-25 hydrocarbon units. In some aspects, the diacyl lipid comprises acyl chains comprising 16-20 hydrocarbon units. In some aspects, the diacyl lipid comprises acyl chains comprising 12, 13, 14, 15, 16, 17, 18, 19, 20, 21, 22, 23, 24, 25, 26, 27, 28, 29 or 30 hydrocarbon units. In some aspects, the diacyl lipid comprises acyl chains comprising 18 hydrocarbon units.

In any of the foregoing or related aspects, the amphiphilic ligand conjugate comprises a CAR ligand operably linked to the lipid via a linker. In some aspects, the linker is selected from the group consisting of hydrophilic polymers, a string of hydrophilic amino acids, polysaccharides, or a combination thereof. In some aspects, the linker comprises “N” consecutive polyethylene glycol units, wherein N is between 25-50.

In other aspects, the disclosure provides an amphiphilic ligand conjugate comprising, a CAR ligand operably linked to a diacyl lipid via a linker, wherein the diacyl lipid comprises acyl chains comprising 12-30 hydrocarbon units, and wherein the linker comprises “N” consecutive polyethylene glycol units, wherein N is between 25-50.

In any of the foregoing or related aspects, the amphiphilic ligand conjugate of the disclosure comprises a CAR ligand that is a tag. In some aspects, the tag is selected from the group consisting of fluorescein isothiocyanate (FITC), streptavidin, biotin, dinitrophenol, peridinin chlorophyll protein complex, green fluorescent protein, phycoerythrin (PE), horse radish peroxidase, palmitoylation, nitrosylation, alkalanine phosphatase, glucose oxidase, and maltose binding protein.

In other aspects, the amphiphilic ligand conjugate comprises a CAR ligand that is a tumor-associated antigen, or a fragment thereof. Exemplary tumor antigens include one or more of CD19, CD20, CD22, k light chain, CD30, CD33, CD123, CD38, ROR1, ErbB2, ErbB3/4, EGFr vIII, carcinoembryonic antigen, EGP2, EGP40, mesothelin, TAG72, PSMA, NKG2D ligands, B7-H6, IL-13 receptor a 2, MUC1, MUC16, CA9, GD2, GD3, HMW-MAA, CD171, Lewis Y, G250/CALX, HLA-AI MAGE A1, HLA-A2 NY-ESO-1, PSC1, folate receptor-α, CD44v7/8, 8H9, NCAM, VEGF receptors, 5T4, Fetal AchR, NKG2D ligands, CD44v6, TEM1, and/or TEM8.

In other aspects, the disclosure provides an amphiphilic ligand conjugate comprising, a lipid operably linked to fluorescein isothiocyanate (FITC) via a polyethylene glycol moiety. In yet other aspects, the disclosure provides an amphiphilic ligand conjugate comprising a lipid operably linked to a fragment of a tumor-associated antigen (e.g., CD19, CD20, CD22, HER2, EGFRvII) via a polyethylene glycol moiety. In some aspects, the lipid is 1,2-distearoyl-sn-glycero-3-phosphoethanolamine (DSPE) and the polyethylene glycol moiety is PEG-2000.

In any of the foregoing or related aspects, the amphiphilic ligand conjugate of the disclosure comprises a lipid, wherein the lipid is 1,2-distearoyl-sn-glycero-3-phosphoethanolamine (DSPE). In some aspects, the amphiphilic ligand conjugate of the disclosure comprises 1,2-distearoyl-sn-glycero-3-phosphoethanolamine (DSPE) linked to a CAR ligand via PEG-2000.

In another aspect, the disclosure provides an amphiphilic ligand conjugate comprising, 1,2-distearoyl-sn-glycero-3-phosphoethanolamine (DSPE) operably linked to fluorescein isothiocyanate (FITC) via a polyethylene glycol moiety. In other aspects, the disclosure provides an amphiphilic ligand conjugate comprising, 1,2-distearoyl-sn-glycero-3-phosphoethanolamine (DSPE) operably linked to fragment of a tumor-associated antigen (e.g., CD19, CD20, CD22, HER2, EGFRvII) via a polyethylene glycol moiety.

In yet other aspects, the disclosure provides an amphiphilic ligand conjugate comprising, 1,2-distearoyl-sn-glycero-3-phosphoethanolamine (DSPE) operably linked to fluorescein isothiocyanate (FITC) via PEG-2000. In yet further aspects, the disclosure provides an amphiphilic ligand conjugate comprising 1,2-distearoyl-sn-glycero-3-phosphoethanolamine (DSPE) operably linked to fragment of a tumor-associated antigen (e.g., CD19, CD20, CD22, HER2, EGFRvII) via PEG-2000.

In any of the foregoing or related aspects, the amphiphilic ligand conjugate of the disclosure comprises a CAR ligand which binds to a CAR, wherein the CAR comprises a co-stimulation domain.

In any of the foregoing or related aspects, the amphiphilic ligand conjugate of the disclosure comprises a CAR ligand which binds to a CAR, wherein the CAR comprises a bispecific binding domain. In some aspects, the bispecific binding domain comprises a tag binding domain and a tumor-associated antigen binding domain (e.g., CD19, CD20, CD22, HER2, EGFRvII). In some aspects, the bispecific binding domain comprises a first tumor-associated antigen binding domain (e.g., CD19, CD20, CD22, HER2, EGFRvII) and a second tumor associated antigen binding domain (e.g., CD19, CD20, CD22, HER2, EGFRvII). In some aspects, the bispecific binding domain comprises a tag binding domain and a tumor-associated antigen binding domain, and wherein the CAR ligand is a tag. In some aspects, the bispecific binding domain comprises a first tumor-associated antigen binding domain and a second tumor-associated antigen binding domain, and wherein the CAR ligand comprises a first or second tumor-associated antigen, or fragment thereof.

In any of the foregoing or related aspects, the amphiphilic ligand conjugate of the disclosure comprises a CAR ligand comprising a tag, and the CAR comprises a tag binding domain. In other aspects, the CAR ligand is a tumor-associated antigen or a fragment thereof, and the CAR comprises a tumor-associated antigen binding domain.

In another aspect, the disclosure provides an amphiphilic ligand conjugate comprising a diacyl lipid operably linked to a tag, wherein the tag binds to a CAR comprising a tag binding domain. In another aspect, the disclosure provides an amphiphilic ligand conjugate comprising a diacyl lipid operably linked to a tag via a polyethylene glycol moiety, wherein the tag binds to a CAR comprising a tag binding domain.

In another aspect, the disclosure provides an amphiphilic ligand conjugate comprising a diacyl lipid operably linked to a tag, wherein the tag binds to a CAR comprising a tag binding domain and a tumor-associated antigen binding domain. In another aspect, the disclosure provides an amphiphilic ligand conjugate comprising a diacyl lipid operably linked to a tag via a polyethylene glycol moiety, wherein the tag binds to a CAR comprising a tag binding domain and a tumor-associated antigen binding domain.

In another aspect, the disclosure provides an amphiphilic ligand conjugate comprising a diacyl lipid operably linked to a tumor-associated antigen or fragment thereof, wherein the tumor-associated antigen binds to a CAR comprising a tumor-associated antigen binding domain (e.g., CD19, CD20, CD22, HER2, EGFRvII). In another aspect, the disclosure provides an amphiphilic ligand conjugate comprising a diacyl lipid operably linked to a tumor-associated antigen or fragment thereof via a polyethylene glycol moiety, wherein the tumor-associated antigen or fragment thereof binds to a CAR comprising a tumor-associated antigen binding domain binding domain. In some aspects, the CAR comprises a first tumor-associated antigen binding domain and a second tumor-associated antigen binding domain, wherein the amphiphilic ligand conjugate comprises either the first or second tumor-associated antigen.

In other aspects, the disclosure provides a composition comprising an amphiphilic ligand conjugate as described herein, and a pharmaceutically acceptable carrier.

In another aspects, the disclosure provides an immunogenic composition comprising a composition as described herein, and an adjuvant.

In some aspects, the immunogenic composition comprises an adjuvant, wherein the adjuvant is an amphiphilic oligonucleotide conjugate comprising an immunostimulatory oligonucleotide conjugated to a lipid, with or without a linker, and optionally a polar compound. In some aspects, the immunostimulatory oligonucleotide binds a pattern recognition receptor. In some aspects, the immunostimulatory oligonucleotide comprises CpG. In some aspects, the immunostimulatory oligonucleotide is a ligand for a toll-like receptor.

In any of the foregoing aspects, the amphiphilic oligonucleotide conjugate comprises a linker, wherein the linker is an oligonucleotide linker. In some aspects, the oligonucleotide linker comprises “N” consecutive guanines, wherein N is between 0-2. In some aspects, the lipid of the amphiphilic oligonucleotide conjugate is a diacyl lipid. In some aspects, the diacyl lipid comprises acyl chains comprising 12-30 hydrocarbon units. In some aspects, the diacyl lipid comprises acyl chains comprising 14-25 hydrocarbon units. In some aspects, the diacyl lipid comprises acyl chains comprising 16-20 hydrocarbon units. In some aspects, the diacyl lipid comprises acyl chains comprising 12, 13, 14, 15, 16, 17, 18, 19, 20, 21, 22, 23, 24, 25, 26, 27, 28, 29 or 30 hydrocarbon units. In some aspects, the diacyl lipid comprises acyl chains comprising 18 hydrocarbon units.

In other aspects, the immunogenic composition comprises an adjuvant, wherein the adjuvant is a cyclic di-GMP (CDG).

In another aspect, the disclosure provides methods of activating, expanding or increasing proliferation of CAR-T cells in a subject, comprising administering to the subject an amphiphilic ligand conjugate, composition or immunogenic composition described herein. In some aspects, the proliferation of CAR(−) T cells is not increased in the subject. In some aspects, the CAR comprises a tag binding domain and the CAR ligand of the amphiphilic ligand conjugate is a tag. In some aspects, the CAR comprises a tumor-associated antigen binding domain and the CAR ligand of the amphiphilic ligand conjugate is a tumor-associated antigen or fragment thereof. In some aspects, the CAR comprises a tag binding domain and a tumor-associated antigen binding domain, and the CAR ligand of the amphiphilic ligand conjugate is a tag. In some aspects, the CAR comprises a first tumor-associated antigen binding domain and a second tumor-associated antigen binding domain, and the CAR ligand of the amphiphilic ligand conjugate is the first or second tumor-associated antigen, or fragment thereof.

In yet other aspects, the disclosure provides methods of reducing or decreasing a size of a tumor or inhibiting a tumor growth in a subject in need thereof, comprising administering to the subject an amphiphilic ligand conjugate, composition or immunogenic composition described herein, wherein the subject is receiving or has received CAR-T cell therapy. In some aspects, the CAR comprises a tag binding domain and the CAR ligand of the amphiphilic ligand conjugate is a tag. In some aspects, the CAR comprises a tumor-associated antigen binding domain and the CAR ligand of the amphiphilic ligand conjugate is a tumor-associated antigen or fragment thereof. In some aspects, the CAR comprises a tag binding domain and a tumor-associated antigen binding domain, and the CAR ligand of the amphiphilic ligand conjugate is a tag. In some aspects, the CAR comprises a first tumor-associated antigen binding domain and a second tumor-associated antigen binding domain, and the CAR ligand of the amphiphilic ligand conjugate is the first or second tumor-associated antigen, or fragment thereof.

In further aspects, the disclosure provides methods of inducing an anti-tumor response in a subject with cancer, comprising administering to the subject an amphiphilic ligand conjugate, composition or immunogenic composition described herein, wherein the subject is receiving or has received CAR-T cell therapy. In some aspects, the CAR comprises a tag binding domain and the CAR ligand of the amphiphilic ligand conjugate is a tag. In some aspects, the CAR comprises a tumor-associated antigen binding domain and the CAR ligand of the amphiphilic ligand conjugate is a tumor-associated antigen or fragment thereof. In some aspects, the CAR comprises a tag binding domain and a tumor-associated antigen binding domain, and the CAR ligand of the amphiphilic ligand conjugate is a tag. In some aspects, the CAR comprises a first tumor-associated antigen binding domain and a second tumor-associated antigen binding domain, and the CAR ligand of the amphiphilic ligand conjugate is the first or second tumor-associated antigen, or fragment thereof.

In another aspects, the disclosure provides methods of stimulating an immune response to a target cell population or target tissue expressing an antigen in a subject, the method comprising administering to the subject CAR-T cells targeted to the antigen, and an amphiphilic ligand conjugate, composition or immunogenic composition described herein. In some aspects the immune response is a T-cell mediated immune response or an anti-tumor immune response. In some aspects, the target cell population or target tissue is tumor cells or tumor tissue. In some aspects, the CAR comprises a tag binding domain and the CAR ligand of the amphiphilic ligand conjugate is a tag. In some aspects, the CAR comprises a tumor-associated antigen binding domain and the CAR ligand of the amphiphilic ligand conjugate is a tumor-associated antigen or fragment thereof. In some aspects, the CAR comprises a tag binding domain and a tumor-associated antigen binding domain, and the CAR ligand of the amphiphilic ligand conjugate is a tag. In some aspects, the CAR comprises a first tumor-associated antigen binding domain and a second tumor-associated antigen binding domain, and the CAR ligand of the amphiphilic ligand conjugate is the first or second tumor-associated antigen, or fragment thereof.

In another aspect, the disclosure provides methods of stimulating an immune response to a target cell population or target tissue expressing an antigen in a subject, the method comprising administering to the subject CAR-T cells targeted to the antigen, and an amphiphilic ligand conjugate, composition or immunogenic composition described herein, wherein the target cell population or target tissue is a population of cells or tissue infected with a virus. In some aspects, the virus is human immunodeficiency virus (HIV). In some aspects, the immune response is a T-cell mediated immune response. In some aspects, the CAR comprises a tag binding domain and the CAR ligand of the amphiphilic ligand conjugate is a tag.

In further aspects, the disclosure provides methods of treating a subject having a disease, disorder or condition associated with expression or elevated expression of an antigen, comprising administering to the subject CAR-T cells targeted to the antigen, and an amphiphilic ligand conjugate, composition or immunogenic composition described herein. In some aspects, the antigen is a viral antigen or caner antigen. In some aspects, the CAR comprises a tag binding domain and the CAR ligand of the amphiphilic ligand conjugate is a tag. In some aspects, the CAR comprises a tumor-associated antigen binding domain and the CAR ligand of the amphiphilic ligand conjugate is a tumor-associated antigen or fragment thereof. In some aspects, the CAR comprises a tag binding domain and a tumor-associated antigen binding domain, and the CAR ligand of the amphiphilic ligand conjugate is a tag.

In any of the foregoing aspects, the method comprises administration of the amphiphilic ligand conjugate, the composition or the immunogenic composition to the subject prior to receiving CAR-T cells. In other aspects, the method comprises administration of the amphiphilic ligand conjugate, the composition or the immunogenic composition to the subject after receiving CAR-T cells. In another aspects, the method comprises administration of the amphiphilic ligand conjugate, the composition or the immunogenic composition to the subject with CAR-T cells administered simultaneously.

In any of the foregoing of related aspects, the amphiphilic ligand conjugate of the disclosure is trafficked to the lymph nodes. In some aspects, the amphiphilic ligand conjugate is trafficked to the inguinal lymph node and auxiliary lymph node. In some aspects, the amphiphilic ligand conjugate is inserted into the membrane of antigen presenting cells upon trafficking to the lymph nodes. In some aspects, the antigen presenting cells are medullary macrophages, CD8+ dendritic cells, and/or CD11b+ dendritic cells.

In any of the foregoing aspects, the CAR ligand is retained in the lymph nodes for at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, or at least 25 days.

In any of the foregoing aspects, wherein the CAR ligand is a tag and the CAR comprises a tag binding domain, the methods further comprise administering a formulation of tagged proteins, and wherein the tag binding domain binds the tagged proteins. In some aspects, the protein of the tagged protein is an antibody or an antigen-binding fragment thereof. In some aspects, the tag binding domain is an antibody or an antigen-binding fragment thereof. In some aspects, the formulation of tagged proteins is administered to the subject prior to administration of the CART cells and amphiphilic ligand conjugate, composition, or immunogenic composition. In other aspects, the formulation of tagged proteins is administered to the subject concurrently with administration of the CAR-T cells and amphiphilic ligand conjugate, composition, or immunogenic composition. In yet other aspects, the formulation of tagged proteins is administered to the subject after administration of the CAR-T cells and amphiphilic ligand conjugate, composition, or immunogenic composition.

In any of the foregoing aspects, the CAR-T cells are administered prior to administration of the amphiphilic ligand conjugate, composition, or immunogenic composition. In other aspects, the CAR-T cells are administered after administration of the amphiphilic ligand conjugate, composition, or immunogenic composition. In yet other aspects, the CAR-T cells are administered concurrently with administration of the amphiphilic ligand conjugate, composition, or immunogenic composition.

In any of the foregoing aspects, an amphiphilic ligand conjugate, composition or immunogenic composition described herein is administered parenterally at a non-tumor draining lymph node, parenterally at a tumor-draining lymph node, or intratumorally.

In any of the foregoing aspects, the subject has cancer. In any of the foregoing aspects, the subject is human.

In another aspect, the disclosure provides a kit comprising a container comprising a composition an amphiphilic ligand conjugate described herein, an optional pharmaceutically acceptable carrier, and a package insert comprising instructions for administration of the composition for treating or delaying progression of cancer in an individual receiving CAR-T cell therapy. In some aspects, the CAR comprises a tag binding domain and the CAR ligand of the amphiphilic ligand conjugate is a tag. In some aspects, the CAR comprises a tumor-associated antigen binding domain and the CAR ligand of the amphiphilic ligand conjugate is a tumor-associated antigen or fragment thereof. In some aspects, the CAR comprises a tag binding domain and a tumor-associated antigen binding domain, and the CAR ligand of the amphiphilic ligand conjugate is a tag. In some aspects, the CAR comprises a first tumor-associated antigen binding domain and a second tumor-associated antigen binding domain, and the CAR ligand of the amphiphilic ligand conjugate is the first or second tumor-associated antigen, or fragment thereof.

In yet other aspects, the disclosure provides a kit comprising a medicament comprising a composition comprising an amphiphilic ligand conjugate described herein, an optional pharmaceutically acceptable carrier, and a package insert comprising instructions for administration of the medicament alone or in combination with a composition comprising an adjuvant and an optional pharmaceutically acceptable carrier, for treating or delaying progression of cancer in an individual receiving CAR-T cell therapy. In some aspects, the CAR comprises a tag binding domain and the CAR ligand of the amphiphilic ligand conjugate is a tag. In some aspects, the CAR comprises a tumor-associated antigen binding domain and the CAR ligand of the amphiphilic ligand conjugate is a tumor-associated antigen or fragment thereof. In some aspects, the CAR comprises a tag binding domain and a tumor-associated antigen binding domain, and the CAR ligand of the amphiphilic ligand conjugate is a tag. In some aspects, the CAR comprises a first tumor-associated antigen binding domain and a second tumor-associated antigen binding domain, and the CAR ligand of the amphiphilic ligand conjugate is the first or second tumor-associated antigen, or fragment thereof.

In other aspects, the disclosure provides a kit comprising a container comprising a composition comprising an amphiphilic ligand conjugate described herein, an optional pharmaceutically acceptable carrier, and a package insert comprising instructions for administration of composition vaccine for activating, expanding or increasing proliferation of CAR-T cells in an individual receiving CAR-T cell therapy. In some aspects, the CAR comprises a tag binding domain and the CAR ligand of the amphiphilic ligand conjugate is a tag. In some aspects, the CAR comprises a tumor-associated antigen binding domain and the CAR ligand of the amphiphilic ligand conjugate is a tumor-associated antigen or fragment thereof. In some aspects, the CAR comprises a tag binding domain and a tumor-associated antigen binding domain, and the CAR ligand of the amphiphilic ligand conjugate is a tag. In some aspects, the CAR comprises a first tumor-associated antigen binding domain and a second tumor-associated antigen binding domain, and the CAR ligand of the amphiphilic ligand conjugate is the first or second tumor-associated antigen, or fragment thereof.

In some aspects, the disclosure provides a kit comprising a medicament comprising a composition comprising an amphiphilic ligand conjugate described herein, an optional pharmaceutically acceptable carrier, and a package insert comprising instructions for administration of the medicament alone or in combination with a composition comprising an adjuvant and an optional pharmaceutically acceptable carrier, for activating, expanding or increasing proliferation of CAR-T cells in an individual receiving CAR-T cell therapy. In some aspects, the CAR comprises a tag binding domain and the CAR ligand of the amphiphilic ligand conjugate is a tag. In some aspects, the CAR comprises a tumor-associated antigen binding domain and the CAR ligand of the amphiphilic ligand conjugate is a tumor-associated antigen or fragment thereof. In some aspects, the CAR comprises a tag binding domain and a tumor-associated antigen binding domain, and the CAR ligand of the amphiphilic ligand conjugate is a tag. In some aspects, the CAR comprises a first tumor-associated antigen binding domain and a second tumor-associated antigen binding domain, and the CAR ligand of the amphiphilic ligand conjugate is the first or second tumor-associated antigen, or fragment thereof.

In any of the foregoing aspects, the kit comprises an adjuvant and instructions for administration of the adjuvant for treating or delaying progression of cancer in an individual receiving CAR-T cell therapy. In some aspects, the adjuvant is an amphiphilic oligonucleotide conjugate comprising an immunostimulatory oligonucleotide as described herein.

In another aspect, the disclosure provides use of an amphiphilic ligand conjugate, composition, or immunogenic composition described herein, for activating, expanding or increasing proliferation of CAR-T cells in an individual receiving CAR-T cell therapy.

In yet other aspects, the disclosure provides use of an amphiphilic ligand conjugate, composition, or immunogenic composition described herein, for treating or delaying progression of cancer in an individual.

In another aspect, the disclosure provides use of an amphiphilic ligand conjugate, composition, or immunogenic composition described herein, in the manufacture of a medicament for treating or delaying progression of cancer in an individual.

In other aspects, the disclosure provides a kit comprising a medicament comprising a composition comprising an amphiphilic ligand conjugate described herein, an optional pharmaceutically acceptable carrier, and a package insert comprising instructions for administration of the composition for treating or delaying progression of a viral infection in an individual receiving CAR-T cell therapy. In some aspects, the kit comprises a formulation of tagged proteins and instructions for administration of the formulation of tagged proteins, wherein the CAR comprises a tag binding domain that binds the tagged proteins. In some aspects, the kit comprises an adjuvant and instructions for administration of the adjuvant for treating or delaying progression of a viral infection in an individual receiving CAR-T cell therapy. In some aspects, the adjuvant is an amphiphilic oligonucleotide conjugate described herein.

### BRIEF DESCRIPTION OF THE DRAWINGS

FIG. 1A provides schematic representations of amphiphilic ligand conjugates comprising a lipid tail (e.g., DSPE) conjugated to a small molecule (top), short linear peptide (middle) or protein domain (bottom) via a PEG-2000 linker.

FIG. 1B provides a schematic illustrating the interaction between an antigen presenting cell decorated with an amphiphilic ligand conjugate comprising a chimeric antigen receptor (CAR) ligand, and a CAR-T cell.

FIG. 2A provides a schematic representation of the domain structure and orientation of a transmembrane anti-FITC CAR.

FIG. 2B provides a graph of flow cytometric data depicting the extent of anti-FITC CAR surface expression following retroviral transduction into primary mouse T cells.

FIG. 2C provides a graph depicting the quantification of IFNγ produced by anti-FITC CAR-T cells following interaction with K562 cells decorated with various concentration of DSPE-PEG-FITC as indicated. ***p<0.0001, **p<0.01, *p<0.05.

FIG. 2D provides a graph depicting the percentage of cell death of DSPE-PEG-FITC coated DC2.4 cells 6 hours after co-culture with FITC-CAR-T cells, at effector to target (E:T) ratio of 10:1. ***p<0.0001, **p<0.01, *p<0.05.

FIG. 3A provides a graph depicting the extent of DSPE-PEG-FITC retention (measured by radiant efficiency) in lymph nodes removed from mice after subsequent days following vaccination with DSPE-PEG-FITC or FITC alone at various doses as indicated.

FIG. 3B provides a graph depicting DSPE-PEG-FITC uptake by different lymphoid populations in draining inguinal lymph nodes 24 hours after subcutaneous injection.

FIG. 3C provides a graph of flow cytometric data depicting the uptake of DSP-PEG-FITC at various doses by three different APCs following subcutaneous injection.

FIG. 4 provides a graph depicting the proliferation index of FITC CAR-T cells in inguinal lymph nodes primed by PBS, c-di-GMP (CDG), DSPE-PEG-FITC or DSPE-PEG-FITC+CDG. The effect of PBS and CDG alone were evaluated one day post vaccination.

FIG. 5 provides a graph depicting DSPE-PEG-FITC display on antigen presenting cell surface, with or without CDG, in lymph node cell populations. Lymph nodes were collected 24 hours and 3 days after DSPE-PEG-FITC vaccination+/−CDG. ***p<0.0001, **p<0.01, *p<0.05.

FIG. 6 provides graphs depicting the mean fluorescence intensity (MFI) of various co-stimulatory molecules on DSPE-PEG-FITC uptaking CD11c+ cells with or without CDG. ***p<0.0001, **p<0.01, *p<0.05.

FIG. 7 provides a schematic depicting an experimental timeline (top) and a graph showing the percentage of CD45.1 FITC CAR-T cells with two rounds of DSPE-PEG-FITC vaccination in lymphodepleted CD45.2 mice (bottom). ***p<0.0001, **p<0.01, *p<0.05.

FIG. 8 provides a schematic depicting an experimental timeline (top) and a graph showing the percentage of CD45.1 FITC CAR-T cells with two rounds of DSPE-PEG-FITC vaccination in lymphreplete CD45.2 mice. ***p<0.0001, **p<0.01, *p<0.05.

FIG. 9 provides a graph showing antibody response over time against repeated DSPE-PEG-FITC vaccination. ***p<0.0001, **p<0.01, *p<0.05.

FIG. 10A provides a schematic showing an EGFRvIII peptide conjugated to DSPE-PEG.

FIG. 10B shows surface expression of EGFRvIII CAR on murine T cells after immunization with DSPE-PEG-EGFRvIII.

FIG. 10C shows proliferation of EGFRvIII CAR T cells in lymph nodes 48 hours after DSPE-PEG-EGFRvIII vaccination as determined by cell trace violet tracking.

FIG. 11A provides a graph depicting the quantification of IFNγ produced by EGFRvIII CAR-T cells or control T cells following interaction with CT-2A glioma cells with or without EGFRvIII expressed on the cell surface. ***p<0.0001, **p<0.01, *p<0.05.

FIG. 11B provides a graph depicting the percentage of cell death of CT-2A glioma cells harboring wildtype EGFR or EGFRvIII after co-culturing with EGFRvIII CAR-T cells or control T cells. ***p<0.0001, **p<0.01, *p<0.05.

FIG. 12 provides a graph depicting the percentage of EGFRvIII CAR T cells in mice that received DSPE-PEG-EGFRvIII (“VAX”) or control vaccination.

FIG. 13 provides a graph showing cytokine (IFNγ and TNFα) secretion of circulating CAR T or non-CAR T cells (n=5) in response to EGFRvIII-expressing target cells with or without DSPE-PEG-EGFRvIII (“VAX”) in vitro.

FIG. 14 provides a schematic depicting the experimental timeline (top) and a graph showing tumor-infiltration of EGFRvIII CAR-T cells as measured by the number of CAR-T cells per mg of tumor in mice implanted with EGFRvIII expressing CT-2A cells and administered DSPE-PEG-EGFRvIII (“PepVIII Vax”).

FIG. 15 provides a graph showing cytokine (IFNγ and TNFα) secretion of tumor infiltrating CAR-T cells in response to PBS or DSPE-PEG-EGFRvIII (“VAX”).

FIG. 16 provides graphs depicting expression level of granzyme B (left) and proliferation as determined by Ki67 (right) of tumor infiltrating CAR-T cells in response to PBS or DSPE-PEG-EGFRvIII (“PepVIII Vax”).

FIG. 17 provides a graph depicting the expression of PD-1 and TIM3 on tumor infiltrating EGFRvIII CAR T cells with or without DSPE-PEG-EGFRvIII (“VAX”).

FIG. 18A provides a graph showing tumor volume in CT-2A tumor bearing mice treated with EGFRvIII CAR-T+/−DSPE-PEG-EGFRvIII vaccination (“VAX”) under lymphodepletion conditions. ***p<0.0001, **p<0.01, *p<0.05.

FIG. 18B provides a Kaplan-Meier survival graph of the CT-2A tumor bearing mice of FIG. 18A.

FIG. 19 provides a schematic of a FITC-antigen bispecific CAR design targeting both FITC and the melanoma-associated antigen TRP1.

FIG. 20 provides a graph depicting FITC-TRP1 CAR expression on T cell surface.

FIG. 21 provides a graph depicting IFNγ secretion of FITC-TRP1 bispecific CAR T upon co-culturing with DSPE-PEG-FITC coated K562 cells or B16F10 cells. Monospecific FITC CAR T cells and TRP1 CAR T cells were included as control. ***p<0.0001, **p<0.01, *p<0.05.

FIG. 22 provides a graph depicting percentage of cell death of TRP1-expressing target cells when co-cultured with FITC-TRP1 bispecific CAR-T or monospecific TRP1 CAR T cells in vitro. Co-culture was set up for 6 hours at effector to target (E:T) ratio of 10:1.

FIG. 23 provides a graph depicting FITC-TRP1 CAR-T proliferation in lymph nodes 48 hours after DSPE-PEG-FITC vaccination as measured by cell trace violet tracking.

FIGS. 24A and 24B show tumor growth (FIG. 24A) and animal survival (FIG. 24B) of B16F10 tumor bearing mice treated with FITC-TRP1 bispecific CAR-T therapy alone or CAR-T plus DSPE-PEG-FITC vaccination (“VAX”) with lymphodepletion preconditioning.

FIG. 25 provides a graph depicting the number of FITC-TRP1 bispecific CAR-T in peripheral blood of mice receiving PBS or DSPE-PEG-FITC vaccination (“VAX”).

FIG. 26 provides a graph depicting the infiltration of FITC/TRP1-CAR T cells into B16F10 tumor in mice receiving PBS or DSPE-PEG-FITC vaccination.

FIGS. 27A and 27B show tumor growth (FIG. 27A) and animal survival (FIG. 27B) of lymphreplete B16F10 tumor bearing mice treated with FITC-TRP1 bispecific CAR-T therapy alone or CAR-T plus DSPE-PEG-FITC vaccination (“VAX”).

### DETAILED DESCRIPTION

Overview

Various diseases are characterized by the development of progressive immunosuppression in a patient. The presence of an impaired immune response in patients with malignancies has been particularly well documented. Cancer patients and tumor-bearing mice exhibit a variety of altered immune functions such as a decrease in delayed type hypersensitivity, a decrease in lytic function and proliferative response of lymphocytes. Augmenting immune functions in cancer patients could have beneficial effects for tumor control.

Chimeric antigen receptor (CAR) T cell therapy has been successful for treating hematologic malignancies. However, CAR-T cells fail to functionally persist in some patients and show generally poor responses in solid tumors. Current protocols for CAR-T therapy rely on infusions of large numbers of CAR-T cells, which can die out or rapidly lose functional activity against tumors. In preclinical animal models, it is known that expanding T cells in vivo through vaccination is one of the most effective strategies for bolstering the efficacy of T cell therapy, but a traditional vaccine cannot boost CAR-T through their chimeric antigen receptor.

Based on the present disclosure, enhancement of CAR-T activation and proliferation is achieved using an amphiphilic ligand conjugate comprising a ligand of the chimeric antigen receptor and a lipid. The amphiphilic ligand conjugates of the disclosure provide a solution to several shortcomings with current approaches toward the generation of therapeutic CAR-T cells by stimulating transferred CAR-T cells in vivo, which may lower the amount of infused CAR-T cells required for a durable therapeutic response and may mitigate the need for patient lymphodepletion.

### Definitions

Terms used in the claims and specification are defined as set forth below unless otherwise specified.

It must be noted that, as used in the specification and the appended claims, the singular forms “a,” “an” and “the” include plural referents unless the context clearly dictates otherwise.

As used herein, “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

As used herein, the term “adjuvant” refers to a compound that, with a specific immunogen or antigen, will augment or otherwise alter or modify the resultant immune response. Modification of the immune response includes intensification or broadening the specificity of either or both antibody and cellular immune responses. Modification of the immune response can also mean decreasing or suppressing certain antigen-specific immune responses. In certain embodiments, the adjuvant is a cyclic dinucleotide. In some embodiments, the adjuvant is an immunostimulatory oligonucleotide as described herein. In some embodiments, the adjuvant is administered prior to, concurrently, or after administration of an amphiphilic ligand conjugate, or composition comprising the conjugate. In some embodiments, the adjuvant is co-formulated in the same composition as an amphiphilic ligand conjugate.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and 0-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

An “amino acid substitution” refers to the replacement of at least one existing amino acid residue in a predetermined amino acid sequence (an amino acid sequence of a starting polypeptide) with a second, different “replacement” amino acid residue. An “amino acid insertion” refers to the incorporation of at least one additional amino acid into a predetermined amino acid sequence. While the insertion will usually consist of the insertion of one or two amino acid residues, the present larger “peptide insertions,” can be made, e.g. insertion of about three to about five or even up to about ten, fifteen, or twenty amino acid residues. The inserted residue(s) may be naturally occurring or non-naturally occurring as disclosed above. An “amino acid deletion” refers to the removal of at least one amino acid residue from a predetermined amino acid sequence.

As used herein, “amphiphile” or “amphiphilic” refers to a conjugate comprising a hydrophilic head group and a hydrophobic tail, thereby forming an amphiphilic conjugate. In some embodiments, an amphiphile conjugate comprises a chimeric antigen receptor (CAR) ligand and one or more hydrophobic lipid tails, referred to herein as an “amphiphilic ligand conjugate.” In some embodiments, the amphiphile conjugate further comprises a polymer (e.g., polyethylene glycol), wherein the polymer is conjugated to the one or more lipids or the CAR ligand.

The term “ameliorating” refers to any therapeutically beneficial result in the treatment of a disease state, e.g., cancer, including prophylaxis, lessening in the severity or progression, remission, or cure thereof.

As used herein, the term “antigenic formulation” or “antigenic composition” or “immunogenic composition” refers to a preparation which, when administered to a vertebrate, especially a mammal, will induce an immune response.

The term “antigen presenting cell” or “APC” is a cell that displays foreign antigen complexed with MHC on its surface. T cells recognize this complex using T cell receptor (TCR). Examples of APCs include, but are not limited to, dendritic cells (DCs), peripheral blood mononuclear cells (PBMC), monocytes (such as THP-1), B lymphoblastoid cells (such as C1R.A2, 1518 B-LCL) and monocyte-derived dendritic cells (DCs). Some APCs internalize antigens either by phagocytosis or by receptor-mediated endocytosis.

As used herein, the term “bispecific” or “bifunctional antibody” refers to an artificial hybrid antibody or fragment thereof having two different heavy/light chain pairs and two different binding sites. Bispecific antibodies can be produced by a variety of methods including fusion of hybridomas or linking of Fab′ fragments. See, e.g., Songsivilai & Lachmann, (1990) Clin. Exp. Immunol. 79:315-321; Kostelny et al., (1992) J. Immunol. 148:1547-1553.

As used herein, the term “chimeric antigen receptor (CAR)” refers to an artificial transmembrane protein receptor comprising (i) an extracellular domain capable of binding to at least one predetermined CAR ligand or antigen, or a predetermined CAR ligand and an antigen, (ii) an intracellular segment comprising one or more cytoplasmic domains derived from signal transducing proteins different from the polypeptide from which the extracellular domain is derived, and (iii) a transmembrane domain. The “chimeric antigen receptor (CAR)” is sometimes called a “chimeric receptor”, a “T-body”, or a “chimeric immune receptor (CIR).”

The phrase “CAR ligand” used interchangeably with “CAR antigen” means any natural or synthetic molecule (e.g., small molecule, protein, peptide, lipid, carbohydrate, nucleic acid) or part or fragment thereof that can specifically bind to a CAR (e.g., the extracellular domain of a CAR). In some embodiments, the CAR ligand is a tumor-associated antigen, or fragment thereof. In some embodiments, the CAR ligand is a tag. One of skill in the art can determine a suitable CAR ligand for use in an amphiphilic ligand conjugate based on the CAR being utilized in a cell therapy.

The “intracellular signaling domain” means any oligopeptide or polypeptide domain known to function to transmit a signal causing activation or inhibition of a biological process in a cell, for example, activation of an immune cell such as a T cell or a NK cell. Examples include ILR chain, CD28 and/or CD3 ζ.

As used herein, “cancer antigen” refers to (i) tumor-specific antigens, (ii) tumor-associated antigens, (iii) cells that express tumor-specific antigens, (iv) cells that express tumor-associated antigens, (v) embryonic antigens on tumors, (vi) autologous tumor cells, (vii) tumor-specific membrane antigens, (viii) tumor-associated membrane antigens, (ix) growth factor receptors, (x) growth factor ligands, and (xi) any other type of antigen or antigen-presenting cell or material that is associated with a cancer.

As used herein, “CG oligodeoxynucleotides (CG ODNs)”, also referred to as “CpG ODNs”, are short single-stranded synthetic DNA molecules that contain a cytosine nucleotide (C) followed by a guanine nucleotide (G). In certain embodiments, the immunostimulatory oligonucleotide is a CG ODN.

As used herein the term “co-stimulatory ligand” includes a molecule on an antigen presenting cell (e.g., an APC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (rCAM), CD30L, CD40, CD70, CD83, HLA-G, MICA, MICB, HVEM, lymphotoxin beta receptor, TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-IBB, OX40, CD30, CD40, PD-1, 1COS, lymphocyte function-associated antigen-1 (LFA-1), CD2, CD7, LIGHT, NKG2C, B7-H3, and a ligand that specifically binds with CD83.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules

A polypeptide or amino acid sequence “derived from” a designated polypeptide or protein refers to the origin of the polypeptide. Preferably, the polypeptide or amino acid sequence which is derived from a particular sequence has an amino acid sequence that is essentially identical to that sequence or a portion thereof, wherein the portion consists of at least 10-20 amino acids, preferably at least 20-30 amino acids, more preferably at least 30-50 amino acids, or which is otherwise identifiable to one of ordinary skill in the art as having its origin in the sequence.

Polypeptides derived from another peptide may have one or more mutations relative to the starting polypeptide, e.g., one or more amino acid residues which have been substituted with another amino acid residue or which has one or more amino acid residue insertions or deletions.

A polypeptide can comprise an amino acid sequence which is not naturally occurring. Such variants necessarily have less than 100% sequence identity or similarity with the starting molecule. In a preferred embodiment, the variant will have an amino acid sequence from about 75% to less than 100% amino acid sequence identity or similarity with the amino acid sequence of the starting polypeptide, more preferably from about 80% to less than 100%, more preferably from about 85% to less than 100%, more preferably from about 90% to less than 100% (e.g., 91%, 92%, 93%, 94%, 95%, 96%, 97%, 98%, 99%) and most preferably from about 95% to less than 100%, e.g., over the length of the variant molecule.

In one embodiment, there is one amino acid difference between a starting polypeptide sequence and the sequence derived therefrom. Identity or similarity with respect to this sequence is defined herein as the percentage of amino acid residues in the candidate sequence that are identical (i.e., same residue) with the starting amino acid residues, after aligning the sequences and introducing gaps, if necessary, to achieve the maximum percent sequence identity.

As used herein, the term antigen “cross-presentation” refers to presentation of exogenous protein antigens to T cells via MHC class I and class II molecules on APCs.

As used herein, the term “cytotoxic T lymphocyte (CTL) response” refers to an immune response induced by cytotoxic T cells. CTL responses are mediated primarily by CD8⁺ T cells.

As used herein, the term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts effective for this use will depend upon the severity of the disorder being treated and the general state of the patient's own immune system.

As used herein, the term “effector cell” or “effector immune cell” refers to a cell involved in an immune response, e.g., in the promotion of an immune effector response. In some embodiments, immune effector cells specifically recognize an antigen. Examples of immune effector cells include, but are not limited to, Natural Killer (NK) cells, B cells, monocytes, macrophages, T cells (e.g., cytotoxic T lymphocytes (CTLs). In some embodiments, the effector cell is a T cell.

As used herein, the term “immune effector function” or “immune effector response” refers to a function or response of an immune effector cell that promotes an immune response to a target.

As used herein, the term “hematological cancer” includes a lymphoma, leukemia, myeloma or a lymphoid malignancy, as well as a cancer of the spleen and lymph nodes. Exemplary lymphomas include both B cell lymphomas (a B-cell hematological cancer) and T cell lymphomas. B-cell lymphomas include both Hodgkin's lymphomas and most non-Hodgkin's lymphomas. Non-limiting examples of B cell lymphomas include diffuse large B-cell lymphoma, follicular lymphoma, mucosa-associated lymphatic tissue lymphoma, small cell lymphocytic lymphoma (overlaps with chronic lymphocytic leukemia), mantle cell lymphoma (MCL), Burkitt's lymphoma, mediastinal large B cell lymphoma, Waldenstrom macroglobulinemia, nodal marginal zone B cell lymphoma, splenic marginal zone lymphoma, intravascular large B-cell lymphoma, primary effusion lymphoma, lymphomatoid granulomatosis. Non-limiting examples of T cell lymphomas include extranodal T cell lymphoma, cutaneous T cell lymphomas, anaplastic large cell lymphoma, and angioimmunoblastic T cell lymphoma. Hematological malignancies also include leukemia, such as, but not limited to, secondary leukemia, chronic lymphocytic leukemia, acute myelogenous leukemia, chronic myelogenous leukemia, and acute lymphoblastic leukemia. Hematological malignancies further include myelomas, such as, but not limited to, multiple myeloma and smoldering multiple myeloma. Other hematological and/or B cell- or T-cell-associated cancers are encompassed by the term hematological malignancy.

As used herein, “immune cell” is a cell of hematopoietic origin and that plays a role in the immune response. Immune cells include lymphocytes (e.g., B cells and T cells), natural killer cells, and myeloid cells (e.g., monocytes, macrophages, eosinophils, mast cells, basophils, and granulocytes).

As used herein, an “immunostimulatory oligonucleotide” is an oligonucleotide that can stimulate (e.g., induce or enhance) an immune response.

The terms “inducing an immune response” and “enhancing an immune response” are used interchangeably and refer to the stimulation of an immune response (i.e., either passive or adaptive) to a particular antigen. The term “induce” as used with respect to inducing CDC or ADCC refer to the stimulation of particular direct cell killing mechanisms.

As used herein, a subject “in need of prevention,” “in need of treatment,” or “in need thereof,” refers to one, who by the judgment of an appropriate medical practitioner (e.g., a doctor, a nurse, or a nurse practitioner in the case of humans; a veterinarian in the case of non-human mammals), would reasonably benefit from a given treatment (such as treatment with a composition comprising an amphiphilic ligand conjugate).

The term “in vivo” refers to processes that occur in a living organism.

As used herein, the terms “linked,” “operably linked,” “fused”, or “fusion”, are used interchangeably. These terms refer to the joining together of two more elements or components or domains, by an appropriate means including chemical conjugation or recombinant DNA technology. Methods of chemical conjugation (e.g., using heterobifunctional crosslinking agents) are known in the art as are methods of recombinant DNA technology.

The term “lipid” refers to a biomolecule that is soluble in nonpolar solvents and insoluble in water. Lipids are often described as hydrophobic or amphiphilic molecules which allows them to form structures such as vesicles or membranes in aqueous environments. Lipids include fatty acids, glycerolipids, glycerophospholipids, sphingolipids, sterol lipids (including cholesterol), prenol lipids, saccharolipids, and polyketides. In some embodiments, the lipid suitable for the amphiphilic ligand conjugates of the disclosure binds to human serum albumin under physiological conditions. In some embodiments, the lipid suitable for the amphiphilic ligand conjugates of the disclosure inserts into a cell membrane under physiological conditions. In some embodiments, the lipid binds albumin and inserts into a cell membrane under physiological conditions. In some embodiments, the lipid is a diacyl lipid. In some embodiments, the diacyl lipid comprises more than 12 carbons. In some embodiments, the diacyl lipid comprises at least 13, at least 14, at least 15, at least 16, at least 17 or at least 18 carbons.

The term “mammal” or “subject” or “patient” as used herein includes both humans and non-humans and includes, but is not limited to, humans, non-human primates, canines, felines, murines, bovines, equines, and porcines.

“Nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19:5081, 1991; Ohtsuka et al., J. Biol. Chem. 260:2605-2608, 1985); and Cassol et al., 1992; Rossolini et al., Mol. Cell. Probes 8:91-98, 1994). For arginine and leucine, modifications at the second base can also be conservative. The term nucleic acid is used interchangeably with gene, cDNA, and mRNA encoded by a gene.

Polynucleotides of the present invention can be composed of any polyribonucleotide or polydeoxribonucleotide, which can be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, the polynucleotide can be composed of triple-stranded regions comprising RNA or DNA or both RNA and DNA. A polynucleotide can also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

In some embodiments, the peptides of the invention are encoded by a nucleotide sequence. Nucleotide sequences of the invention can be useful for a number of applications, including: cloning, gene therapy, protein expression and purification, mutation introduction, DNA vaccination of a host in need thereof, antibody generation for, e.g., passive immunization, PCR, primer and probe generation, and the like.

As used herein, “parenteral administration,” “administered parenterally,” and other grammatically equivalent phrases, refer to modes of administration other than enteral and topical administration, usually by injection, and include, without limitation, intravenous, intranasal, intraocular, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intracerebral, intracranial, intracarotid and intrasternal injection and infusion.

As generally used herein, “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

As used herein, the term “physiological conditions” refers to the in vivo condition of a subject. In some embodiments, physiological condition refers to a neutral pH (e.g., pH between 6-8).

“Polypeptide,” “peptide”, and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

As used herein, a “small molecule” is a molecule with a molecular weight below about 500 Daltons.

As used herein, the term “subject” includes any human or non-human animal. For example, the methods and compositions of the present invention can be used to treat a subject with a cancer or infection. The term “non-human animal” includes all vertebrates, e.g., mammals and non-mammals, such as non-human primates, sheep, dog, cow, chickens, amphibians, reptiles, etc.

The term “sufficient amount” or “amount sufficient to” means an amount sufficient to produce a desired effect, e.g., an amount sufficient to reduce the diameter of a tumor.

The term “T cell” refers to a type of white blood cell that can be distinguished from other white blood cells by the presence of a T cell receptor on the cell surface. There are several subsets of T cells, including, but not limited to, T helper cells (a.k.a. T_{H} cells or CD4⁺ T cells) and subtypes, including T_{H}1, T_{H}2, T_{H}3, T_{H}17, T_{H}9, and T_{FH} cells, cytotoxic T cells (i.e., T_{C} cells, CD8⁺ T cells, cytotoxic T lymphocytes, T-killer cells, killer T cells), memory T cells and subtypes, including central memory T cells (T_{CM} cells), effector memory T cells (T_{EM} and T_{EMRA} cells), and resident memory T cells (T_{RM} cells), regulatory T cells (a.k.a. T_{reg} cells or suppressor T cells) and subtypes, including CD4+FOXP3⁺ T_{reg} cells, CD4⁺FOXP3⁻ T_{reg} cells, Tr1 cells, Th3 cells, and T_{reg}17 cells, natural killer T cells (a.k.a. NKT cells), mucosal associated invariant T cells (MAITs), and gamma delta T cells (γδ T cells), including Vγ9/Vδ2 T cells. Any one or more of the aforementioned or unmentioned T cells may be the target cell type for a method of use of the invention.

As used herein, the term “T cell activation” or “activation of T cells” refers to a cellular process in which mature T cells, which express antigen-specific T cell receptors on their surfaces, recognize their cognate antigens and respond by entering the cell cycle, secreting cytokines or lytic enzymes, and initiating or becoming competent to perform cell-based effector functions. T cell activation requires at least two signals to become fully activated. The first occurs after engagement of the T cell antigen-specific receptor (TCR) by the antigen-major histocompatibility complex (MHC), and the second by subsequent engagement of co-stimulatory molecules (e.g., CD28). These signals are transmitted to the nucleus and result in clonal expansion of T cells, upregulation of activation markers on the cell surface, differentiation into effector cells, induction of cytotoxicity or cytokine secretion, induction of apoptosis, or a combination thereof.

As used herein, the term “T cell-mediated response” refers to any response mediated by T cells, including, but not limited to, effector T cells (e.g., CD8⁺ cells) and helper T cells (e.g., CD4⁺ cells). T cell mediated responses include, for example, T cell cytotoxicity and proliferation.

The term “T cell cytotoxicity” includes any immune response that is mediated by CD8+ T cell activation. Exemplary immune responses include cytokine production, CD8+ T cell proliferation, granzyme or perforin production, and clearance of an infectious agent.

A “therapeutic antibody” is an antibody, fragment of an antibody, or construct that is derived from an antibody, and can bind to a cell-surface antigen on a target cell to cause a therapeutic effect. Such antibodies can be chimeric, humanized or fully human antibodies. Methods are known in the art for producing such antibodies. Such antibodies include single chain Fc fragments of antibodies, minibodies and diabodies. Any of the therapeutic antibodies known in the art to be useful for cancer therapy can be used in combination therapy with the compositions described herein. Therapeutic antibodies may be monoclonal antibodies or polyclonal antibodies. In preferred embodiments, the therapeutic antibodies target cancer antigens. In some embodiments, a therapeutic antibody comprises a tag binding domain, which is recognized by an amphiphilic ligand conjugate comprising a tag.

As used herein, “therapeutic protein” refers to any polypeptide, protein, protein variant, fusion protein and/or fragment thereof which may be administered to a subject as a medicament.

The term “therapeutically effective amount” is an amount that is effective to ameliorate a symptom of a disease. A therapeutically effective amount can be a “prophylactically effective amount” as prophylaxis can be considered therapy.

The terms “treat,” “treating,” and “treatment,” as used herein, refer to therapeutic or preventative measures described herein. The methods of “treatment” employ administration to a subject, in need of such treatment, an amphiphilic ligand conjugate of the present disclosure, for example, a subject receiving CAR T cell therapy. In some embodiments, an amphiphilic ligand conjugate is administered to a subject in need of an enhanced immune response against a particular antigen or a subject who ultimately may acquire such a disorder, in order to prevent, cure, delay, reduce the severity of, or ameliorate one or more symptoms of the disorder or recurring disorder, or in order to prolong the survival of a subject beyond that expected in the absence of such treatment.

As used herein, “vaccine” refers to a formulation which contains an amphiphilic ligand conjugate as described herein, combined with an adjuvant, which is in a form that is capable of being administered to a vertebrate and which induces a protective immune response sufficient to induce immunity to prevent and/or ameliorate an infection or disease and/or to reduce at least one symptom of an infection or disease and/or to enhance the efficacy of another dose of the synthetic nanoparticle. Typically, the vaccine comprises a conventional saline or buffered aqueous solution medium in which a composition as described herein is suspended or dissolved. In this form, a composition as described herein is used to prevent, ameliorate, or otherwise treat an infection or disease. Upon introduction into a host, the vaccine provokes an immune response including, but not limited to, the production of antibodies and/or cytokines and/or the activation of cytotoxic T cells, antigen presenting cells, helper T cells, dendritic cells and/or other cellular responses.

Chimeric Antigen Receptors

In some aspects, the disclosure provides compositions and methods to be used or performed in conjunction with chimeric antigen receptor (CAR) effector cells.

Chimeric antigen receptors (CARs) are genetically-engineered, artificial transmembrane receptors, which confer an arbitrary specificity for a ligand onto an immune effector cell (e.g. a T cell, natural killer cell or other immune cell) and which results in activation of the effector cell upon recognition and binding to the ligand. Typically these receptors are used to impart the antigen specificity of a monoclonal antibody onto a T cell.

In some embodiments, CARs contain three domains: 1) an ectodomain typically comprising a signal peptide, a ligand or antigen recognition region (e.g. scFv), and a flexible spacer; 2) a transmembrane (TM) domain; 3) an endodomain (alternatively known as an “activation domain”) typically comprising one or more intracellular signaling domains. The ectodomain of the CAR resides outside of the cell and is exposed to the extracellular space, whereby it is accessible for interaction with its cognate ligand. The TM domain allows the CAR to be anchored into the cell membrane of the effector cell. The third endodomain (also known as the “activation domain”) aids in effector cell activation upon binding of the CAR to its specific ligand. In some embodiments, effector cell activation comprises induction of cytokine and chemokine production, as well as activation of the cytolytic activity of the cells. In some embodiments, the CARs redirect cytotoxicity toward tumor cells.

In some embodiments, CARs comprise a ligand- or antigen-specific recognition domain that binds to a specific target ligand or antigen (also referred to as a binding domain). In some embodiments, the binding domain is a single-chain antibody variable fragment (scFv), a tethered ligand or the extracellular domain of a co-receptor, fused to a transmembrane domain, which is linked, in turn, to a signaling domain. In some embodiments, the signaling domain is derived from CD3 or FcRy. In some embodiments, the CAR comprises one or more co-stimulatory domains derived from a protein such as CD28, CD137 (also known as 4-1BB), CD134 (also known as OX40) and CD278 (also known as ICOS).

Engagement of the antigen binding domain of the CAR with its target antigen on the surface of a target cell results in clustering of the CAR and delivers an activation stimulus to the CAR-containing cell. In some embodiments, the main characteristic of CARs are their ability to redirect immune effector cell specificity, thereby triggering proliferation, cytokine production, phagocytosis or production of molecules that can mediate cell death of the target antigen expressing cell in a major histocompatibility (MHC) independent manner, exploiting the cell specific targeting abilities of monoclonal antibodies, soluble ligands or cell specific co-receptors. Although scFv-based CARs engineered to contain a signaling domain from CD3 or FcRy have been shown to deliver a potent signal for T cell activation and effector function, they are not sufficient to elicit signals that promote T cell survival and expansion in the absence of a concomitant co-stimulatory signal. A new generation of CARs containing a binding domain, a hinge, a transmembrane and the signaling domain derived from CD3 or FcRy together with one or more co-stimulatory signaling domains (e.g., intracellular co-stimulatory domains derived from CD28, CD137, CD134 and CD278) has been shown to more effectively direct antitumor activity as well as increased cytokine secretion, lytic activity, survival and proliferation in CAR expressing T cells in vitro, in animal models and cancer patients (Milone et al., Molecular Therapy, 2009; 17: 1453-1464; Zhong et al., Molecular Therapy, 2010; 18: 413-420; Carpenito et al., PNAS, 2009; 106:3360-3365).

In some embodiments, chimeric antigen receptor-expressing effector cells (e.g. CAR-T cells) are cells that are derived from a patient with a disease or condition and genetically modified in vitro to express at least one CAR with an arbitrary specificity to a ligand. The cells perform at least one effector function (e.g. induction of cytokines) that is stimulated or induced by the specific binding of the ligand to the CAR and that is useful for treatment of the same patient's disease or condition. The effector cells may be T cells (e.g. cytotoxic T cells or helper T cells). One skilled in the art would understand that other cell types (e.g. a natural killer cell or a stem cell) may express CARs and that a chimeric antigen receptor effector cell may comprise an effector cell other than a T cell. In some embodiments, the effector cell is a T cell (e.g. a cytotoxic T cell) that exerts its effector function (e.g. a cytotoxic T cell response) on a target cell when brought in contact or in proximity to the target or target cell (e.g. a cancer cell) (see e.g., Chang and Chen (2017) Trends Mol Med 23(5):430-450).

Prolonged exposure of T cells to their cognate antigen can result in exhaustion of effector functions, enabling the persistence of infected or transformed cells. Recently developed strategies to stimulate or rejuvenate host effector function using agents that induce an immune checkpoint blockade have resulted in success towards the treatment of several cancers. Emerging evidence suggests that T cell exhaustion may also represent a significant impediment in sustaining long-lived antitumor activity by chimeric antigen receptor-expressing T cells (CAR-T cells. In some embodiments, the differentiation status of the patient-harvested T cells prior to CAR transduction and the conditioning regimen a patient undergoes before reintroducing the CAR-T cells (e.g., addition or exclusion of alkylating agents, fludarabine, total-body irradiation) can profoundly affect the persistence and cytotoxic potential of CAR-T cells. In vitro culture conditions that stimulate (via anti-CD3/CD28 or stimulator cells) and expand (via cytokines, such as IL-2) T cell populations can also alter the differentiation status and effector function of CAR-T cells (Ghoneim et al., (2016) Trends in Molecular Medicine 22(12):1000-1011).

The present disclosure addresses several shortcomings with current approaches toward the generation of therapeutic CAR-T cells. Existing methods of therapeutic CAR-T cell preparation often requires extensive cell culture in vitro to obtain a sufficient number of modified cells for adoptive cell transfer, during which natural identity or differentiation state of the T cells may have changed and T cell function may have been compromised. Furthermore, when patients are in urgent need of therapy to prevent disease progression, the time required to generate sufficient quantities of CAR-T cells may not be aligned with the opportunity to treat the patient, resulting in therapeutic failure and demise of the patient. The compositions and methods provided by the disclosure bypass this hurdle and offer an expedient and more physiologically relevant therapeutic approach by stimulating CAR-T cell activation and proliferation in vivo. In addition, current CAR-T cell therapy regime requires lymphodepletion beforehand, which weakens patients' health and destroys the nourishing environment that can improve CAR-T efficacy. In some aspects, the disclosure provides methods to stimulate adoptively transferred CAR-T cells such that they can still engraft, actively proliferate and expand in vivo in the absence of lymphodepletion.

Current CAR-T cell therapy only relies on the engineered co-stimulatory signal to maintain CAR-T effector function. The lack of other co-stimulatory signals and a natural stimulatory environment may lead to incomplete T cell maturation and increased T cell exhaustion. In one aspect, the disclosure provides methods and compositions to recruit T cells into lymph nodes, the physiologically relevant activation environment for immune cells and co-administration of adjuvant to activate APCs which provide a complete suite of essential co-stimulatory signals for optimal CAR-T cell activation.

In some embodiments, in particular for the treatment of ALL and/or NHL, suitable CARs target CD19 or CD20. Non-limiting examples include CARs comprising a structure: (i) an anti-CD19 scFv, a CD8 H/TM domain, an 4-1BB CS domain and a CD3ζ TCR signaling domain; (ii) an anti-CD19 scFv, a CD28 hinge and transmembrane domain, a CD28 co-stimulatory domain and a CD3ζ TCR signaling domain; and (iii) an anti-CD20 scFv, an IgG hinge and transmembrane domain, a CD28/4-1BB co-stimulatory domain and a CD3ζ TCR signaling domain. In some embodiments, a CAR effector cell suitable for combination with the combinations and methods disclosed herein targets CD19 or CD20, including but not limited to Kymriah™ (tisagenlecleucel; Novartis; formerly CTL019) and Yescarta™ (axicabtagene ciloleucel; Kite Pharma).

Re-Targeted CAR T Cells

In some embodiments, effector cells (e.g., T cells) modified to express a CAR which binds to a universal immune receptor, a tag, a switch or an Fc region on an immunoglobulin are suitable for the compositions and methods described herein.

In some embodiments, effector cells (e.g., T cells) are modified to express a universal immune receptor or UnivlR. One type of UnivlR is a biotin-binding immune receptor (BBIR) (see e.g., US Patent Publication US20140234348 A1 incorporated herein by reference in its entirety). Other examples of methods and compositions relating to universal chimeric receptors and/or effector cells expressing universal chimeric receptors are described in International Patent Applications WO2016123122A1, WO2017143094A1, WO2013074916A1, US Patent Application US20160348073A1, all of which are incorporated herein by reference in their entirety.

In some embodiments, effector cells (e.g., T cells) are modified to express a universal, modular, anti-tag chimeric antigen receptor (UniCAR). This system allows for retargeting of UniCAR engrafted immune cells against multiple antigens (see e.g., US Patent Publication US20170240612 A1 incorporated herein by reference in its entirety; Cartellieri et al., (2016) Blood Cancer Journal 6, e458 incorporated herein by reference in its entirety).

In some embodiments, effector cells (e.g., T cells) are modified to express a switchable chimeric antigen receptor and chimeric antigen receptor effector cell (CAR-EC) switches. In this system, the CAR-EC switches have a first region that is bound by a chimeric antigen receptor on the CAR-EC and a second region that binds a cell surface molecule on target cell, thereby stimulating an immune response from the CAR-EC that is cytotoxic to the bound target cell. In some embodiments, the CAR-EC is a T cell, wherein the CAR-EC switch may act as an “on-switch” for CAR-EC activity. Activity may be “turned off” by reducing or ceasing administration of the switch. These CAR-EC switches may be used with CAR-ECs disclosed herein, as well as existing CAR T-cells, for the treatment of a disease or condition, such as cancer, wherein the target cell is a malignant cell. Such treatment may be referred to herein as switchable immunotherapy (US Patent Publication U.S. Pat. No. 9,624,276 B2 incorporated herein by reference in its entirety).

In some embodiments, effector cells (e.g., T cells) are modified to express a receptor that binds the Fc portion of human immunoglobulins (e.g., CD16V-BB-ζ) (Kudo et al., (2014) Cancer Res 74(1):93-103 incorporated herein by reference in its entirety).

In some embodiments, effector cells (e.g., T cells) are modified to express a universal immune receptor (e.g., switchable CAR, sCAR) that binds a peptide neo-epitope (PNE). In some embodiments, the peptide neo-epitope (PNE), has been incorporated at defined different locations within an antibody targeting an antigen (antibody switch). Therefore, sCAR-T-cell specificity is redirected only against PNE, not occurring in the human proteome, thus allowing an orthogonal interaction between the sCAR-T-cell and the antibody switch. In this way, sCAR-T cells are strictly dependent on the presence of the antibody switch to become fully activated, thus excluding CAR T-cell off-target recognition of endogenous tissues or antigens in the absence of the antibody switch (Arcangeli et al., (2016) Transl Cancer Res 5(Suppl 2):S174-S177 incorporated herein by reference in its entirety). Other examples of switchable CARs is provided by US Patent Application US20160272718A1 incorporated herein by reference in its entirety.

As used herein, the term “tag” encompasses a universal immune receptor, a tag, a switch, or an Fc region of an immunoglobulin as described supra. In some embodiments, an effector cell is modified to express a CAR comprising a tag binding domain. In some embodiments, the CAR binds fluorescein isothiocyanate (FITC), streptavidin, biotin, dinitrophenol, peridinin chlorophyll protein complex, green fluorescent protein, phycoerythrin (PE), horse radish peroxidase, palmitoylation, nitrosylation, alkalanine phosphatase, glucose oxidase, or maltose binding protein.

Anti-TAG Chimeric Antigen Receptors (AT-CAR)

There are several limitations to the generalized clinical application of CAR T cells. For example, as there is no single tumor antigen universally expressed by all cancer types, each scFv in a CAR needs to be engineered with specificity for the desired tumor antigen. In addition, tumor antigens targeted by a CAR may be down-regulated or mutated in response to treatment resulting in tumor evasion.

As an alternative, universal, anti-tag chimeric antigen receptors (AT-CAR) and CAR-T cells have been developed. For example, human T cells have been engineered to express an anti-fluorescein isothiocyanate (FITC) CAR (referred to anti-FITC-CAR). This platform takes advantage of the high affinity interaction between the anti-FITC scFv (on the cell's surface) and FITC as well as the ability conjugate FITC molecules (or other tags) to any anti-cancer-based monoclonal antibody such as cetuximab (anti-EGFR), retuximab (anti-CD20) and herceptin (anti-Her2).

Accordingly, in some embodiments, effector cells (e.g., T cells) are modified to express a universal anti-tag chimeric antigen receptor (AT-CAR), as described at least in WO 2012082841 and US20160129109A1, incorporated herein by reference in its entirety. In such AT-CAR systems, T cells recognize and bind tagged proteins, such as antibodies. For example, in some embodiments an AT-CAR T cell recognizes tag-labeled antibodies, such as FITC-labeled antibodies. In some embodiments, an anti-tumor antigen antibody is conjugated to a tag (e.g., FITC), and administered prior to, concurrently, or after AT-CAR therapy. Anti-tumor antigen antibodies are known to those of skill in the art.

As indicated, the binding specificity of the tag-binding domain depends on the identity of the tag that is conjugated to the protein that is used to bind target cells. For example, in some aspects of the disclosure, the tag is FITC, the tag-binding domain is an anti-FITC scFv. Alternatively, in some aspects of the disclosure, the tag is biotin or PE (phycoerythrin) and the tag-binding domain is an anti-biotin scFv or an anti-PE scFv.

In some embodiments, the protein of each formulation of tagged proteins is the same or different and the protein is an antibody or an antigen-binding fragment thereof. In some aspects, the antibody or antigen-binding fragment thereof is cetuximab (anti-EGFR), nimotuzumab (anti-EGFR), panitumumab (anti-EGFR), retuximab (anti-CD20), omalizumab (anti-CD20), tositumomab (anti-CD20), trastuzumab (anti-Her2), gemtuzumab (anti-CD33), alemtuzumab (anti-CD52), and bevacuzimab (anti-VEGF).

Thus, in some embodiments, the tagged proteins include FITC-conjugated antibodies, biotin-conjugated antibodies, PE-conjugated antibodies, histidine-conjugated antibodies and streptavidin-conjugated antibodies, where the antibody binds to a TAA or a TSA expressed by the target cells. For example, the tagged proteins include, but are not limited to, FITC-conjugated cetuximab, FITC-conjugated retuximab, FITC-conjugated herceptin, biotin-conjugated cetuximab, biotin-conjugated retuximab, biotin-conjugated herceptin, PE-conjugated cetuximab, PE-conjugated retuximab, PE-conjugated herceptin, histidine-conjugated cetuximab, histidine-conjugated retuximab, histidine-conjugated herceptin, streptavidin-conjugated cetuximab, streptavidin-conjugated retuximab, and streptavidin-conjugated herceptin.

In some embodiments, the AT-CAR of each population of AT-CAR-expressing T cells is the same or different and the AT-CAR comprises a tag-binding domain, a transmembrane domain, and an activation domain. In some embodiments, the tag-binding domain is an antibody or an antigen-binding fragment thereof. In some aspects, the tag-binding domain specifically binds FITC, biotin, PE, histidine or streptavidin. In some embodiments the tag-binding domain is antigen-binding fragment and the antigen-binding fragment is a single chain variable fragment (scFv), such as a scFv that specifically binds FITC, biotin, PE, histidine or streptavidin. In some embodiments the transmembrane domain is the hinge and transmembrane regions of the human CD8a chain. In some embodiments, the activation domain comprises one or more of the cytoplasmic region of CD28, the cytoplasmic region of CD137 (41BB), OX40, HVEM, CD3ζ and FcRε.

In some embodiments, the tag of each formulation of tagged proteins is the same or different and the tag is selected from the group consisting of fluorescein isothiocyanate (FITC), streptavidin, biotin, histidine, dinitrophenol, peridinin chlorophyll protein complex, green fluorescent protein, phycoerythrin (PE), horse radish peroxidase, palmitoylation, nitrosylation, alkalanine phosphatase, glucose oxidase, and maltose binding protein.

The tag may be conjugated to the proteins using techniques such as chemical coupling and chemical cross-linkers. Alternatively, polynucleotide vectors can be prepared that encode the tagged proteins as fusion proteins. Cell lines can then be engineered to express the tagged proteins, and the tagged proteins can be isolated from culture media, purified and used in the methods disclosed herein.

In some embodiments, tagged proteins are administered to a subject prior to, or concurrent with, or after administration of the AT-CAR-expressing T cells. In some embodiments, the disclosure provide a method of treating cancer in a subject, comprising: (a) administering a formulation of tagged proteins to a subject in need of treatment, wherein the tagged proteins bind a cancer cell in the subject, and (b) administering a therapeutically-effective population of anti-tag chimeric antigen receptor (AT-CAR)-expressing T cells to the subject, wherein the AT-CAR-expressing T cells bind the tagged proteins and induce cancer cell death, thereby treating cancer in a subject.

Tandem CAR (Tan CAR) Effector Cells

It has been observed that using a CAR approach for cancer treatment, tumor heterogeneity and immunoediting can cause escape from CAR treatment (Grupp et al., New Eng. J. Med (2013) 368:1509-1518). As an alternative approach, bispecific CARs, known as tandem CARs or TanCARs, have been developed in an attempt to target multiple cancer specific markers simultaneously. In a TanCAR, the extracellular domain comprises two antigen binding specificities in tandem, joined by a linker. The two binding specificities (scFvs) are thus both linked to a single transmembrane portion: one scFv being juxtaposed to the membrane and the other being in a distal position. As an exemplary TanCAR, Grada et al. (Mol Ther Nucleic Acids (2013) 2, e105) describes a TanCAR which includes a CD19-specific scFv, followed by a Gly-Ser linker and a HER2-specific scFv. The HER2-scFv was in the juxta-membrane position, and the CD19-scFv in the distal position. The TanCAR was shown to induce distinct T cell reactivity against each of the two tumor restricted antigens.

Accordingly, some aspects of the disclosure relate to a tandem chimeric antigen receptor that mediates bispecific activation and targeting of T cells. Although the present disclosure refers to bispecificity for the CAR, in some aspects the CARs are able to target three, four, or more tumor antigens. Targeting multiple antigens using CAR T cells may enhance T cell activation and/or offset tumor escape by antigen loss. TanCARs may also target multiple expressed antigens, target various tumors using the same cellular product with a broad specificity, and/or provide a better toxicity profile with a less intensely signaling CAR achieving the same results due to multiple specificity.

In some embodiments, the disclosure provides a TanCAR that includes two targeting domains. In some embodiments, the disclosure provides a multispecific TanCAR that includes three or more targeting domains. In another embodiment, the disclosure provides a first CAR and second CAR at the cell surface, each CAR comprising an antigen-binding domain, wherein the antigen-binding domain of the first CAR binds to a first tumor antigen (e.g., CD19, CD20, CD22, HER2) and the antigen-binding domain of the second CAR binds to another (different) tumor antigen. TanCARs are described in US20160303230A1 and US20170340705A1, incorporated herein by reference.

In some embodiments, the TanCAR of the disclosure targets two or more tumor antigens. Exemplary tumor antigens include one or more of CD19, CD20, CD22, k light chain, CD30, CD33, CD123, CD38, ROR1, ErbB2, ErbB3/4, EGFr vIII, carcinoembryonic antigen, EGP2, EGP40, mesothelin, TAG72, PSMA, NKG2D ligands, B7-H6, IL-13 receptor a 2, MUC1, MUC16, CA9, GD2, GD3, HMW-MAA, CD171, Lewis Y, G250/CALX, HLA-AI MAGE A1, HLA-A2 NY-ESO-1, PSC1, folate receptor-α, CD44v7/8, 8H9, NCAM, VEGF receptors, 5T4, Fetal AchR, NKG2D ligands, CD44v6, TEM1, and/or TEM8.

In some embodiments, the disclosure provides a bispecific TanCAR that targets CD19 and another tumor antigen. In some embodiments, the disclosure provides a bispecific TanCAR that targets CD22 and another tumor antigen. In some embodiments, the disclosure provides a bispecific TanCAR that targets HER2 and another tumor antigen. In some embodiments, the disclosure provides a bispecific TanCAR that targets IL13R-alpha2 and another tumor antigen. In some embodiments, the disclosure provides a bispecific TanCAR that targets VEGF-A and another tumor antigen. In some embodiments, the disclosure provides a bispecific TanCAR that targets Tem8 and another tumor antigen. In some embodiments, the disclosure provides a bispecific TanCAR that targets FAP and another tumor antigen. In some embodiments, the disclosure provides a bispecific TanCAR that targets EphA2 and another tumor antigen. In some embodiments, the disclosure provides a bispecific TanCAR that targets one or more, two or more, three or more, or four or more of the following tumor antigens: CD19, CD22, HER2, IL13R-alpha2, VEGF-A, Tem8, FAP, or EphA2, and any combination thereof. In some embodiments, the disclosure provides a bispecific TanCAR that targets HER2 and IL13R-alpha2. In some embodiments, the disclosure provides a bispecific TanCAR that targets CD19 and CD22.

Methods for Generating Chimeric Antigen Receptors and CAR Effector Cells

In some embodiments, a subject's effectors cells (e.g., T cells) are genetically modified with a chimeric antigen receptor (Sadelain et al., Cancer Discov. 3:388-398, 2013). For example, an effector cell (e.g., T cell) is provided and a recombinant nucleic acid encoding a chimeric antigen receptor is introduced into the patient-derived effector cell (e.g., T cell) to generate a CAR cell. In some embodiments, effector cells (e.g., T cells) not derived from the subject are genetically modified with a chimeric antigen receptor. For example, in some embodiments, effector cells (e.g., T cells) are allogeneic cells that have been engineered to be used as an “off the shelf” adoptive cell therapy, such as Universal Chimeric Antigen Receptor T cells (UCARTs), as developed by Cellectis. UCARTs are allogeneic CAR T cells that have been engineered to be used for treating the largest number of patients with a particular cancer type. Non-limiting examples of UCARTs under development by Cellectis include those that target the following tumor antigens: CD19, CD123, CD22, CS1 and CD38.

A variety of different methods known in the art can be used to introduce any of the nucleic acids or expression vectors disclosed herein into an effector cell (e.g., T cell). Non-limiting examples of methods for introducing nucleic acid into a an effector cell (e.g., T cell) include: lipofection, transfection (e.g., calcium phosphate transfection, transfection using highly branched organic compounds, transfection using cationic polymers, dendrimer-based transfection, optical transfection, particle-based transfection (e.g., nanoparticle transfection), or transfection using liposomes (e.g., cationic liposomes)), microinjection, electroporation, cell squeezing, sonoporation, protoplast fusion, impalefection, hydrodynamic delivery, gene gun, magnetofection, viral transfection, and nucleofection. Furthermore, the CRISPR/Cas9 genome editing technology known in the art can be used to introduce CAR nucleic acids into effector cells (e.g., T cells) and/or to introduce other genetic modifications (e.g., as described below) into effector cells (e.g., T cells) to enhance CAR cell activity (for use of CRISPR/Cas9 technology in connection with CAR T cells, see e.g., U.S. Pat. Nos. 9,890,393; 9,855,297; US 2017/0175128; US 2016/0184362; US 2016/0272999; WO 2015/161276; WO 2014/191128; CN 106755088; CN 106591363; CN 106480097; CN 106399375; CN 104894068).

Provided herein are methods that can be used to generate any of the cells or compositions described herein where each cell can express a CAR (e.g., any of the CARs described herein).

Chimeric antigen receptors (CARs) include an antigen-binding domain, a transmembrane domain, and an cytoplasmic signaling domain that includes a cytoplasmic sequence of CD3 sequence sufficient to stimulate a T cell when the antigen-binding domain binds to the antigen, and optionally, a cytoplasmic sequence of one or more (e.g., two, three, or four) co-stimulatory proteins (e.g., a cytoplasmic sequence of one or more of B7-H3, BTLA, CD2, CD7, CD27, CD28, CD30, CD40, CD40L, CD80, CD160, CD244, ICOS, LAGS, LFA-1, LIGHT, NKG2C, 4-1BB, OX40, PD-1, PD-L1, TIM3, and a ligand that specifically binds to CD83) that provides for co-stimulation of the T cell when the antigen-binding domain binds to the antigen. In some embodiments, a CAR can further include a linker. Non-limiting aspects and features of CARs are described below. Additional aspects of CARs and CAR cells, including exemplary antigen-binding domains, linkers, transmembrane domains, and cytoplasmic signaling domains, are described in, e.g., Kakarla et al., Cancer J. 20:151-155, 2014; Srivastava et al., Trends Immunol. 36:494-502, 2015; Nishio et al., Oncoimmunology 4(2): e988098, 2015; Ghorashian et al., Br. J. Haematol. 169:463-478, 2015; Levine, Cancer Gene Ther. 22:79-84, 2015; Jensen et al., Curr. Opin. Immunol. 33:9-15, 2015; Singh et al., Cancer Gene Ther. 22:95-100, 2015; Li et al., Zhongguo Shi Yan Xue Ye Xue Za Zhi 22:1753-1756, 2014; Gill et al., Immunol. Rev. 263:68-89, 2015; Magee et al., Discov. Med. 18:265-271, 2014; Gargett et al., Front. Pharmacol. 5:235, 2014; Yuan et al., Zhongguo Shi Yan Xue Ye Xue Za Zhi 22:1137-1141, 2014; Pedgram et al., Cancer J. 20:127-133, 2014; Eshhar et al., Cancer J. 20:123-126, 2014; Ramos et al., Cancer J. 20:112-118, 2014; Maus et al., Blood 123:2625-2635, 2014; Jena et al., Curr. Hematol. Malig. Rep. 9:50-56, 2014; Maher et al., Curr. Gene Ther. 14:35-43, 2014; Riches et al., Discov. Med. 16:295-302, 2013; Cheadle et al., Immunol. Rev. 257:83-90, 2014; Davila et al., Int. J. Hematol. 99:361-371, 2014; Xu et al., Cancer Lett. 343:172-178, 2014; Kochenderfer et al., Nat. Rev. Clin. Oncol. 10:267-276, 2013; Hosing et al., Curr. Hematol. Malig. Rep. 8:60-70, 2013; Hombach et al., Curr. Mol. Med. 13:1079-1088, 2013; Xu et al., Leuk. Lymphoma 54:255-260, 2013; Gilham et al., Trends Mol. Med. 18:377-384, 2012; Lipowska-Bhalla et al., Cancer Immunol. Immunother. 61:953-962, 2012; Chmielewski et al., Cancer Immunol. Immunother. 61:1269-1277, 2013; Jena et al., Blood 116:1035-1044, 2010; Dotti et al, Immunology Reviews 257(1): 107-126, 2013; Dai et al., Journal of the National Cancer Institute 108(7): djv439, 2016; Wang and Riviere, Molecular Therapy-Oncolytics 3: 16015, 2016; U.S. Patent Application Publication Nos. 2018/0057609; 2018/0037625; 2017/0362295; 2017/0137783; 2016/0152723, 2016/0206656, 2016/0199412, 2016/0208018, 2015/0232880, 2015/0225480; 2015/0224143; 2015/0224142; 2015/0190428; 2015/0196599; 2015/0152181; 2015/0140023; 2015/0118202; 2015/0110760; 2015/0099299; 2015/0093822; 2015/0093401; 2015/0051266; 2015/0050729; 2015/0024482; 2015/0023937; 2015/0017141; 2015/0017136; 2015/0017120; 2014/0370045; 2014/0370017; 2014/0369977; 2014/0349402; 2014/0328812; 2014/0322275; 2014/0322216; 2014/0322212; 2014/0322183; 2014/0314795; 2014/0308259; 2014/0301993; 2014/0296492; 2014/0294784; 2014/0286973; 2014/0274909; 2014/0274801; 2014/0271635; 2014/0271582; 2014/0271581; 2014/0271579; 2014/0255363; 2014/0242701; 2014/0242049; 2014/0227272; 2014/0219975; 2014/0170114; 2014/0134720; 2014/0134142; 2014/0120622; 2014/0120136; 2014/0106449; 2014/0106449; 2014/0099340; 2014/0086828; 2014/0065629; 2014/0050708; 2014/0024809; 2013/0344039; 2013/0323214; 2013/0315884; 2013/0309258; 2013/0288368; 2013/0287752; 2013/0287748; 2013/0280221; 2013/0280220; 2013/0266551; 2013/0216528; 2013/0202622; 2013/0071414; 2012/0321667; 2012/0302466; 2012/0301448; 2012/0301447; 2012/0060230; 2011/0213288; 2011/0158957; 2011/0104128; 2011/0038836; 2007/0036773; and 2004/0043401. Additional aspects of CARs and CAR cells, including exemplary antigen-binding domains, linkers, transmembrane domains, and cytoplasmic signaling domains, are described in WO 2016/168595; WO 12/079000; 2015/0141347; 2015/0031624; 2015/0030597; 2014/0378389; 2014/0219978; 2014/0206620; 2014/0037628; 2013/0274203; 2013/0225668; 2013/0116167; 2012/0230962; 2012/0213783; 2012/0093842; 2012/0071420; 2012/0015888; 2011/0268754; 2010/0297093; 2010/0158881; 2010/0034834; 2010/0015113; 2009/0304657; 2004/0043401; 2014/0322253; 2015/0118208; 2015/0038684; 2014/0024601; 2012/0148552; 2011/0223129; 2009/0257994; 2008/0160607; 2008/0003683; 2013/0121960; 2011/0052554; and 2010/0178276.

A. Antigen Binding Domains

Antigen binding domains included in the chimeric antigen receptor (CAR) can specifically bind to an antigen (e.g., a tumor associated antigen (TAA) or an antigen that is not expressed on an non-cancerous cell) or a universal receptor (e.g., a tag). Non-limiting examples of an antigen binding domain include: a monoclonal antibody (e.g., IgG1, IgG2, IgG3, IgG4, IgM, IgE, and IgD) (e.g., a fully human or a chimeric (e.g., a humanized) antibody), an antigen binding fragment of an antibody (e.g., Fab, Fab′, or F(ab′)2 fragments) (e.g., a fragment of a fully human or a chimeric (e.g., humanized) antibody), a diabody, a triabody, a tetrabody, a minibody, a scFv, scFv-Fc, (scFv)₂, scFab, bis-scFv, hc-IgG, a BiTE, a single domain antibody (e.g., a V-NAR domain or a VhH domain), IgNAR, and a multispecific (e.g., bispecific antibody) antibody. Methods of making these antigen-binding domains are known in the art.

In some embodiments, an antigen binding domain includes at least one (e.g., one, two, three, four, five, or six) CDR (e.g., any of the three CDRs from an immunoglobulin light chain variable domain or any of the three CDRs from an immunoglobulin heavy chain variable domain) of an antibody that is capable of specifically binding to the target antigen, such as immunoglobulin molecules (e.g., light or heavy chain immunoglobulin molecules) and immunologically-active (antigen-binding) fragments of immunoglobulin molecules.

In some embodiments, an antigen binding domain is a single-chain antibody (e.g., a V-NAR domain or a V_{H}H domain, or any of the single-chain antibodies as described herein). In some embodiments, an antigen binding domain is a whole antibody molecule (e.g., a human, humanized, or chimeric antibody) or a multimeric antibody (e.g., a bi-specific antibody).

In some embodiments, antigen-binding domains include antibody fragments and multispecific (e.g., bi-specific) antibodies or antibody fragments. Examples of antibodies and antigen-binding fragments thereof include, but are not limited to: single-chain Fvs (scFvs), Fab fragments, Fab′ fragments, F(ab′)2, disulfide-linked Fvs (sdFvs), Fvs, and fragments containing either a VL or a VH domain.

Additional antigen binding domains provided herein are polyclonal, monoclonal, multispecific (multimeric, e.g., bi-specific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG₁, IgG₂, IgG₃, IgG₄, IgA₁, and IgA₂), or subclass. In some embodiments, the antigen binding domain is an IgG₁ antibody or antigen-binding fragment thereof. In some examples, the antigen binding domain is an IgG4 antibody or antigen-binding fragment thereof. In some embodiments, the antigen binding domain is an immunoglobulin comprising a heavy and light chain.

Additional examples of antigen binding domains are antigen-binding fragments of an IgG (e.g., an antigen-binding fragment of IgG1, IgG2, IgG3, or IgG4) (e.g., an antigen-binding fragment of a human or humanized IgG, e.g., human or humanized IgG1, IgG2, IgG3, or IgG4), an antigen-binding fragment of an IgA (e.g., an antigen-binding fragment of IgA1 or IgA2) (e.g., an antigen-binding fragment of a human or humanized IgA, e.g., a human or humanized IgA1 or IgA2), an antigen-binding fragment of an IgD (e.g., an antigen-binding fragment of a human or humanized IgD), an antigen-binding fragment of an IgE (e.g., an antigen-binding fragment of a human or humanized IgE), or an antigen-binding fragment of an IgM (e.g., an antigen-binding fragment of a human or humanized IgM).

In some embodiments, an antigen binding domain can bind to a particular antigen (e.g., a tumor-associated antigen) with an affinity (K_{D}) about or less than 1×10⁻⁷ M (e.g., about or less than 1×10⁻⁸ M, about or less than 5×10⁻⁹ M, about or less than 2×10⁻⁹ M, or about or less than 1×10⁻⁹ M), e.g., in saline or in phosphate buffered saline.

As can be appreciated by those in the art, the choice of the antigen binding domain to include in the CAR depends upon the type and number of ligands that define the surface of a cell (e.g., cancer cell or tumor) to be targeted in a subject in need thereof, and/or depends on the ligand present on the amphiphilic ligand conjugate. For example, in some embodiments the antigen binding domain is chosen to recognize a ligand that acts as a cell surface marker on cancer cells, or is a tumor-associated antigen (e.g., CD19, CD30, Her2/neu, EGFR or BCMA) or a tumor-specific antigen (TSA). In some embodiments, the antigen binding domain recognizes a ligand on the amphiphilic ligand conjugate.

In some embodiments, CAR effector cells (e.g., CAR T cells) comprise a CAR molecule that binds to a tumor antigen (e.g., comprises a tumor antigen binding domain). In some embodiments, the CAR molecule comprises an antigen binding domain that recognizes a tumor antigen of a solid tumor (e.g., breast cancer, colon cancer, etc.). In some embodiments, the CAR molecule is a tandem CAR molecule as described supra, which comprises at least two antigen binding domains. In some embodiments, the CAR molecule comprises an antigen binding domain that recognizes a tumor antigen of a hematologic malignancy (e.g., leukemia, acute lymphocytic leukemia, acute myelocytic leukemia, acute promyelocytic leukemia, chronic leukemia, chronic myelocytic (granulocytic) leukemia, chronic lymphocytic leukemia, mantle cell lymphoma, primary central nervous system lymphoma, Burkitt's lymphoma and marginal zone B cell lymphoma, Polycythemia vera, Hodgkin's disease, non-Hodgkin's disease, multiple myeloma, etc.).

In some embodiments, the tumor antigen is a tumor-specific antigen (TSA). A TSA is unique to tumor cells and does not occur on other cells in the body. In some embodiments, the tumor antigen is a tumor-associated antigen (TAA). A TAA is not unique to a tumor cell and instead is also expressed on a normal cell under conditions that fail to induce a state of immunologic tolerance to the antigen. The expression of the antigen on the tumor may occur under conditions that enable the immune system to respond to the antigen. In some embodiments, a TAA is expressed on normal cells during fetal development when the immune system is immature and unable to respond or is normally present at extremely low levels on normal cells but which are expressed at much higher levels on tumor cells.

In certain embodiments, the tumor-associated antigen is determined by sequencing a patient's tumor cells and identifying mutated proteins only found in the tumor. These antigens are referred to as “neoantigens.” Once a neoantigen has been identified, therapeutic antibodies can be produced against it and used in the methods described herein.

In some embodiments, the tumor antigen is an epithelial cancer antigen, (e.g., breast, gastrointestinal, lung), a prostate specific cancer antigen (PSA) or prostate specific membrane antigen (PSMA), a bladder cancer antigen, a lung (e.g., small cell lung) cancer antigen, a colon cancer antigen, an ovarian cancer antigen, a brain cancer antigen, a gastric cancer antigen, a renal cell carcinoma antigen, a pancreatic cancer antigen, a liver cancer antigen, an esophageal cancer antigen, a head and neck cancer antigen, or a colorectal cancer antigen. In certain embodiments, the tumor antigen is a lymphoma antigen (e.g., non-Hodgkin's lymphoma or Hodgkin's lymphoma), a B-cell lymphoma cancer antigen, a leukemia antigen, a myeloma (e.g., multiple myeloma or plasma cell myeloma) antigen, an acute lymphoblastic leukemia antigen, a chronic myeloid leukemia antigen, or an acute myelogenous leukemia antigen.

Tumor antigens, (e.g. tumor-associated antigens (TAAs) and tumor-specific antigens (TSAs)) that may be targeted by CAR effector cells (e.g., CAR T cells), include, but are not limited to, 1GH-IGK, 43-9F, 5T4, 791Tgp72, acyclophilin C-associated protein, alpha-fetoprotein (AFP), α-actinin-4, A3, antigen specific for A33 antibody, ART-4, B7, Ba 733, BAGE, BCR-ABL, beta-catenin, beta-HCG, BrE3-antigen, BCA225, BTAA, CA125, CA15-3\CA 27.29\BCAA, CA195, CA242, CA-50, CAM43, CAMEL, CAP-1, carbonic anhydrase IX, c-Met, CA19-9, CA72-4, CAM 17.1, CASP-8/m, CCCL19, CCCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD68, CD70, CD70L, CD74, CD79a, CD79b, CD80, CD83, CD95, CD126, CD132, CD133, CD138, CD147, CD154, CDC27, CDK4, CDK4m, CDKN2A, CO-029, CTLA4, CXCR4, CXCR7, CXCL12, HIF-la, colon-specific antigen-p (CSAp), CEA (CEACAM5), CEACAM6, c-Met, DAM, E2A-PRL, EGFR, EGFRvIII, EGP-1 (TROP-2), EGP-2, ELF2-M, Ep-CAM, fibroblast growth factor (FGF), FGF-5, Flt-1, Flt-3, folate receptor, G250 antigen, Ga733VEpCAM, GAGE, gp100, GRO-0, H4-RET, HLA-DR, HM1.24, human chorionic gonadotropin (HCG) and its subunits, HER2/neu, HMGB-1, hypoxia inducible factor (HIF-1), HSP70-2M, HST-2, HTgp-175, Ia, IGF-1R, IFN-γ, IFN-α, IFN-β, IFN-k, IL-4R, IL-6R, IL-13R, IL-15R, IL-17R, IL-18R, IL-2, IL-6, IL-8, IL-12, IL-15, IL-17, IL-18, IL-23, IL-25, insulin-like growth factor-1 (IGF-1), KC4-antigen, KSA, KS-1-antigen, KS1-4, LAGE-la, Le-Y, LDR/FUT, M344, MA-50, macrophage migration inhibitory factor (MIF), MAGE, MAGE-1, MAGE-3, MAGE-4, MAGE-5, MAGE-6, MART-1, MART-2, TRAG-3, mCRP, MCP-1, MIP-1A, MIP-1B, MIF, MG7-Ag, MOV18, MUC1, MUC2, MUC3, MUC4, MUC5ac, MUC13, MUC16, MUM-1/2, MUM-3, MYL-RAR, NB/70K, Nm23H1, NuMA, NCA66, NCA95, NCA90, NY-ESO-1, p15, p16, p185erbB2, p180erbB3, PAM4 antigen, pancreatic cancer mucin, PD1 receptor (PD-1), PD-1 receptor ligand 1 (PD-L1), PD-1 receptor ligand 2 (PD-L2), PI5, placental growth factor, p53, PLAGL2, Pmel17 prostatic acid phosphatase, PSA, PRAME, PSMA, P1GF, ILGF, ILGF-1R, IL-6, IL-25, RCAS1, RS5, RAGE, RANTES, Ras, T101, SAGE, S100, survivin, survivin-2B, SDDCAG16, TA-90\Mac2 binding protein, TAAL6, TAC, TAG-72, TLP, tenascin, TRAIL receptors, TRP-1, TRP-2, TSP-180, TNF-α, Tn antigen, Thomson-Friedenreich antigens, tumor necrosis antigens, tyrosinase, VEGFR, ED-B fibronectin, WT-1, 17-1A-antigen, complement factors C3, C3a, C3b, C5a, C5, an angiogenesis marker, bcl-2, bcl-6, and K-ras, an oncogene marker and an oncogene product (see, e.g., Sensi et al., Clin Cancer Res 2006, 12:5023-32; Parmiani et al., J Immunol 2007, 178:1975-79; Novellino et al. Cancer Immunol Immunother 2005, 54:187-207).

In some embodiments, the tumor antigen is a viral antigen derived from a virus associated with a human chronic disease or cancer (such as cervical cancer). For example, in some embodiments, the viral antigen is derived from Epstein-Barr virus (EBV), HPV antigens E6 and/or E7, hepatitis C virus (HCV), hepatitis B virus (HBV), or cytomegalovirus (CMV).

Exemplary cancers or tumors and specific tumor antigens associated with such tumors (but not exclusively), include acute lymphoblastic leukemia (etv6, amll, cyclophilin b), B cell lymphoma (Ig-idiotype), glioma (E-cadherin, α-catenin, β-catenin, γ-catenin, p120ctn), bladder cancer (p21ras), biliary cancer (p21ras), breast cancer (MUC family, HER2/neu, c-erbB-2), cervical carcinoma (p53, p21ras), colon carcinoma (p21ras, HER2/neu, c-erbB-2, MUC family), colorectal cancer (Colorectal associated antigen (CRC)-CO17-1A/GA733, APC), choriocarcinoma (CEA), epithelial cell cancer (cyclophilin b), gastric cancer (HER2/neu, c-erbB-2, ga733 glycoprotein), hepatocellular cancer (α-fetoprotein), Hodgkins lymphoma (Imp-1, EBNA-1), lung cancer (CEA, MAGE-3, NY-ESO-1), lymphoid cell-derived leukemia (cyclophilin b), melanoma (p5 protein, gp75, oncofetal antigen, GM2 and GD2 gangliosides, Melan-A/MART-1, cdc27, MAGE-3, p21ras, gp100), mycloma (MUC family, p21ras), non-small cell lung carcinoma (HER2/neu, c-erbB-2), nasopharyngeal cancer (Imp-1, EBNA-1), ovarian cancer (MUC family, HER2/neu, c-erbB-2), prostate cancer (Prostate Specific Antigen (PSA) and its antigenic epitopes PSA-1, PSA-2, and PSA-3, PSMA, HER2/neu, c-erbB-2, ga733 glycoprotein), renal cancer (HER2/neu, c-erbB-2), squamous cell cancers of the cervix and esophagus, testicular cancer (NY-ESO-1), and T cell leukemia (HTLV-1 epitopes), and viral products or proteins.

In some embodiments, the immune effector cell comprising a CAR molecule (e.g., CAR T cell) useful in the methods disclosed herein expresses a CAR comprising a mesothelin binding domain (i.e., the CAR T cell specifically recognizes mesothelin). Mesothelin is a tumor antigen that is overexpressed in a variety of cancers including ovarian, lung and pancreatic cancers.

In some embodiments, the immune effector cell comprising a CAR molecule (e.g., CAR T cell) useful in the methods disclosed herein expresses a CAR comprising a CD19 binding domain. In some embodiments, the immune effector cell comprising a CAR molecule (e.g., CAR T cell) useful in the methods disclosed herein expresses a CAR comprising a HER2 binding domain. In some embodiments, the immune effector cell comprising a CAR molecule (e.g., CAR T cell) useful in the methods disclosed herein expresses a CAR comprising a EGFR binding domain.

In some embodiments, the CAR effector cell expressing a CAR comprising a CD19 targeting or binding domain is Kymriah™ (tisagenlecleucel; Novartis; see WO 2016109410, herein incorporated by reference in its entirety) or Yescarta™ (axicabtagene ciloleucel; Kite; see US 20160346326, herein incorporated by reference in its entirety).

B. Linker

Provided herein are CARs that can optionally include a linker (1) between the antigen binding domain and the transmembrane domain, and/or (2) between the transmembrane domain and the cytoplasmic signaling domain. In some embodiments, the linker can be a polypeptide linker. For example, the linker can have a length of between about 1 amino acid and about 500 amino acids, about 400 amino acids, about 300 amino acids, about 200 amino acids, about 100 amino acids, about 90 amino acids, about 80 amino acids, about 70 amino acids, about 60 amino acids, about 50 amino acids, about 40 amino acids, about 35 amino acids, about 30 amino acids, about 25 amino acids, about 20 amino acids, about 18 amino acids, about 16 amino acids, about 14 amino acids, about 12 amino acids, about 10 amino acids, about 8 amino acids, about 6 amino acids, about 4 amino acids, or about 2 amino acids; about 2 amino acids to about 500 amino acids, about 400 amino acids, about 300 amino acids, about 200 amino acids, about 100 amino acids, about 90 amino acids, about 80 amino acids, about 70 amino acids, about 60 amino acids, about 50 amino acids, about 40 amino acids, about 35 amino acids, about 30 amino acids, about 25 amino acids, about 20 amino acids, about 18 amino acids, about 16 amino acids, about 14 amino acids, about 12 amino acids, about 10 amino acids, about 8 amino acids, about 6 amino acids, or about 4 amino acids; about 4 amino acids to about 500 amino acids, about 400 amino acids, about 300 amino acids, about 200 amino acids, about 100 amino acids, about 90 amino acids, about 80 amino acids, about 70 amino acids, about 60 amino acids, about 50 amino acids, about 40 amino acids, about 35 amino acids, about 30 amino acids, about 25 amino acids, about 20 amino acids, about 18 amino acids, about 16 amino acids, about 14 amino acids, about 12 amino acids, about 10 amino acids, about 8 amino acids, or about 6 amino acids; about 6 amino acids to about 500 amino acids, about 400 amino acids, about 300 amino acids, about 200 amino acids, about 100 amino acids, about 90 amino acids, about 80 amino acids, about 70 amino acids, about 60 amino acids, about 50 amino acids, about 40 amino acids, about 35 amino acids, about 30 amino acids, about 25 amino acids, about 20 amino acids, about 18 amino acids, about 16 amino acids, about 14 amino acids, about 12 amino acids, about 10 amino acids, or about 8 amino acids; about 8 amino acids to about 500 amino acids, about 400 amino acids, about 300 amino acids, about 200 amino acids, about 100 amino acids, about 90 amino acids, about 80 amino acids, about 70 amino acids, about 60 amino acids, about 50 amino acids, about 40 amino acids, about 35 amino acids, about 30 amino acids, about 25 amino acids, about 20 amino acids, about 18 amino acids, about 16 amino acids, about 14 amino acids, about 12 amino acids, or about 10 amino acids; about 10 amino acids to about 500 amino acids, about 400 amino acids, about 300 amino acids, about 200 amino acids, about 100 amino acids, about 90 amino acids, about 80 amino acids, about 70 amino acids, about 60 amino acids, about 50 amino acids, about 40 amino acids, about 35 amino acids, about 30 amino acids, about 25 amino acids, about 20 amino acids, about 18 amino acids, about 16 amino acids, about 14 amino acids, or about 12 amino acids; about 12 amino acids to about 500 amino acids, about 400 amino acids, about 300 amino acids, about 200 amino acids, about 100 amino acids, about 90 amino acids, about 80 amino acids, about 70 amino acids, about 60 amino acids, about 50 amino acids, about 40 amino acids, about 35 amino acids, about 30 amino acids, about 25 amino acids, about 20 amino acids, about 18 amino acids, about 16 amino acids, or about 14 amino acids; about 14 amino acids to about 500 amino acids, about 400 amino acids, about 300 amino acids, about 200 amino acids, about 100 amino acids, about 90 amino acids, about 80 amino acids, about 70 amino acids, about 60 amino acids, about 50 amino acids, about 40 amino acids, about 35 amino acids, about 30 amino acids, about 25 amino acids, about 20 amino acids, about 18 amino acids, or about 16 amino acids; about 16 amino acids to about 500 amino acids, about 400 amino acids, about 300 amino acids, about 200 amino acids, about 100 amino acids, about 90 amino acids, about 80 amino acids, about 70 amino acids, about 60 amino acids, about 50 amino acids, about 40 amino acids, about 35 amino acids, about 30 amino acids, about 25 amino acids, about 20 amino acids, or about 18 amino acids; about 18 amino acids to about 500 amino acids, about 400 amino acids, about 300 amino acids, about 200 amino acids, about 100 amino acids, about 90 amino acids, about 80 amino acids, about 70 amino acids, about 60 amino acids, about 50 amino acids, about 40 amino acids, about 35 amino acids, about 30 amino acids, about 25 amino acids, or about 20 amino acids; about 20 amino acids to about 500 amino acids, about 400 amino acids, about 300 amino acids, about 200 amino acids, about 100 amino acids, about 90 amino acids, about 80 amino acids, about 70 amino acids, about 60 amino acids, about 50 amino acids, about 40 amino acids, about 35 amino acids, about 30 amino acids, or about 25 amino acids; about 25 amino acids to about 500 amino acids, about 400 amino acids, about 300 amino acids, about 200 amino acids, about 100 amino acids, about 90 amino acids, about 80 amino acids, about 70 amino acids, about 60 amino acids, about 50 amino acids, about 40 amino acids, about 35 amino acids, or about 30 amino acids; about 30 amino acids to about 500 amino acids, about 400 amino acids, about 300 amino acids, about 200 amino acids, about 100 amino acids, about 90 amino acids, about 80 amino acids, about 70 amino acids, about 60 amino acids, about 50 amino acids, about 40 amino acids, or about 35 amino acids; about 35 amino acids to about 500 amino acids, about 400 amino acids, about 300 amino acids, about 200 amino acids, about 100 amino acids, about 90 amino acids, about 80 amino acids, about 70 amino acids, about 60 amino acids, about 50 amino acids, or about 40 amino acids; about 40 amino acids to about 500 amino acids, about 400 amino acids, about 300 amino acids, about 200 amino acids, about 100 amino acids, about 90 amino acids, about 80 amino acids, about 70 amino acids, about 60 amino acids, or about 50 amino acids; about 50 amino acids to about 500 amino acids, about 400 amino acids, about 300 amino acids, about 200 amino acids, about 100 amino acids, about 90 amino acids, about 80 amino acids, about 70 amino acids, or about 60 amino acids; about 60 amino acids to about 500 amino acids, about 400 amino acids, about 300 amino acids, about 200 amino acids, about 150 amino acids, about 100 amino acids, about 90 amino acids, about 80 amino acids, or about 70 amino acids; about 70 amino acids to about 500 amino acids, about 400 amino acids, about 300 amino acids, about 200 amino acids, about 100 amino acids, about 90 amino acids, or about 80 amino acids; about 80 amino acids to about 500 amino acids, about 400 amino acids, about 300 amino acids, about 200 amino acids, about 100 amino acids, or about 90 amino acids; about 90 amino acids to about 500 amino acids, about 400 amino acids, about 300 amino acids, about 200 amino acids, or about 100 amino acids; about 100 amino acids to about 500 amino acids, about 400 amino acids, about 300 amino acids, or about 200 amino acids; about 200 amino acids to about 500 amino acids, about 400 amino acids, or about 300 amino acids; about 300 amino acids to about 500 amino acids or about 400 amino acids; or about 400 amino acids to about 500 amino acids.

Additional examples and aspects of linkers are described in the references cited herein, and are thus incorporated in their entirety herein.

C. Transmembrane Domains

In some embodiments, the CARs described herein also include a transmembrane domain. In some embodiments, the transmembrane domain is naturally associated with a sequence in the cytoplasmic domain. In some embodiments, the transmembrane domain can be modified by one or more (e.g., two, three, four, five, six, seven, eight, nine, or ten) amino acid substitutions to avoid the binding of the domain to other transmembrane domains (e.g., the transmembrane domains of the same or different surface membrane proteins) to minimize interactions with other members of the receptor complex.

In some embodiments, the transmembrane domain may be derived from a natural source. In some embodiments, the transmembrane domain may be derived from any membrane-bound or transmembrane protein. Non-limiting examples of transmembrane domains that may be used herein may be derived from (e.g., comprise at least the transmembrane sequence or a part of the transmembrane sequence of) the alpha, beta, or zeta chain of the T-cell receptor, CD28, CD3 epsilon, CD33, CD37, CD64, CD80, CD45, CD4, CD5, CD8, CD9, CD16, CD22, CD86, CD134, CD137 or CD154.

In some embodiments, the transmembrane domain may be synthetic. For example, in some embodiments where the transmembrane domain is from a synthetic source, the transmembrane domain may include (e.g., predominantly include) hydrophobic residues (e.g., leucine and valine). In some embodiments, the synthetic transmembrane domain will include at least one (e.g., at least two, at least three, at least four, at least five, or at least six) triplet of phenylalanine, tryptophan, and valine at the end of a synthetic transmembrane domain. In some embodiments, the transmembrane domain of a CAR can include a CD8 hinge domain.

Additional specific examples of transmembrane domains are described in the references cited herein.

D. Cytoplasmic Domains

Also provided herein are CAR molecules that comprise, e.g., a cytoplasmic signaling domain that includes a cytoplasmic sequence of CD3 sufficient to stimulate a T cell when the antigen binding domain binds to the antigen, and optionally, a cytoplasmic sequence of one or more of co-stimulatory proteins (e.g., a cytoplasmic sequence of one or more of CD27, CD28, 4-1BB, OX40, CD30, CD40L, CD40, PD-1, PD-L1, ICOS, LFA-1, CD2, CD7, CD160, LIGHT, BTLA, TIM3, CD244, CD80, LAG3, NKG2C, B7-H3, a ligand that specifically binds to CD83, and any of the ITAM sequences described herein or known in the art) that provides for co-stimulation of the T cell. The stimulation of a CAR immune effector cell can result in the activation of one or more anti-cancer activities of the CAR immune effector cell. For example, in some embodiments, stimulation of a CAR immune effector cell can result in an increase in the cytolytic activity or helper activity of the CAR immune effector cell, including the secretion of cytokines. In some embodiments, the entire intracellular signaling domain of a co-stimulatory protein is included in the cytoplasmic signaling domain. In some embodiments, the cytoplasmic signaling domain includes a truncated portion of an intracellular signaling domain of a co-stimulatory protein (e.g., a truncated portion of the intracellular signaling domain that transduces an effector function signal in the CAR immune effector cell). Non-limiting examples of intracellular signaling domains that can be included in a cytoplasmic signaling domain include the cytoplasmic sequences of the T cell receptor (TCR) and co-receptors that act in concert to initiate signal transduction following antigen receptor engagement, as well as any variant of these sequences including at least one (e.g., one, two, three, four, five, six, seven, eight, nine, or ten) substitution and have the same or about the same functional capability.

In some embodiments, a cytoplasmic signaling domain can include two distinct classes of cytoplasmic signaling sequences: signaling sequences that initiate antigen-dependent activation through the TCR (primary cytoplasmic signaling sequences) (e.g., a CD3ζ cytoplasmic signaling sequence) and a cytoplasmic sequence of one or more of co-stimulatory proteins that act in an antigen-independent manner to provide a secondary or co-stimulatory signal (secondary cytoplasmic signaling sequences).

In some embodiments, the cytoplasmic domain of a CAR can be designed to include the CD3ζ signaling domain by itself or combined with any other desired cytoplasmic signaling sequence(s) useful in the context of a CAR. In some examples, the cytoplasmic domain of a CAR can include a CD3ζ chain portion and a costimulatory cytoplasmic signaling sequence. The costimulatory cytoplasmic signaling sequence refers to a portion of a CAR including a cytoplasmic signaling sequence of a costimulatory protein (e.g., CD27, CD28, 4-IBB (CD 137), OX40, CD30, CD40, PD-1, ICOS, lymphocyte function-associated antigen-1 (LFA-1), CD2, CD7, LIGHT, NKG2C, B7-H3, and a ligand that specifically binds with CD83).

In some embodiments, the cytoplasmic signaling sequences within the cytoplasmic signaling domain of a CAR are positioned in a random order. In some embodiments, the cytoplasmic signaling sequences within the cytoplasmic signaling domain of a CAR are linked to each other in a specific order. In some embodiments, a linker (e.g., any of the linkers described herein) can be used to form a linkage between different cytoplasmic signaling sequences.

In some embodiments, the cytoplasmic signaling domain is designed to include the cytoplasmic signaling sequence of CD3 and the cytoplasmic signaling sequence of the costimulatory protein CD28. In some embodiments, the cytoplasmic signaling domain is designed to include the cytoplasmic signaling sequence of CD3 and the cytoplasmic signaling sequence of costimulatory protein 4-IBB. In some embodiments, the cytoplasmic signaling domain is designed to include the cytoplasmic signaling sequence of CD3ζ and the cytoplasmic signaling sequences of costimulatory proteins CD28 and 4-1BB. In some embodiments, the cytoplasmic signaling domain does not include the cytoplasmic signaling sequences of 4-1BB.

Additional Modification of CAR T Cells

In another embodiment, the therapeutic efficacy of CAR effector cells (e.g., CAR T cel is enhanced by disruption of a methylcytosine dioxygenase gene (e.g., Tet1, Tet2, Tet3), which leads to decreased total levels of 5-hydroxymethylcytosine in association with enhanced proliferation, regulation of effector cytokine production and degranulation, and thereby increases CAR effector cell (e.g., CAR T cell) proliferation and/or function, as described in PCT Publication WO 2017/049166. Thus, an effector cell (e.g., T cell) can be engineered to express a CAR and wherein expression and/or function of Tet1, Tet2 and/or Tet1 in said effector cell (e.g., T cell) has been reduced or eliminated.

In another embodiment, the therapeutic efficacy of CAR effector cells (e.g., CAR T cells) is enhanced by using an effector cell (e.g., T cell) that constitutively expresses a CAR (referred to as a nonconditional CAR) and conditionally expresses another agent useful for treating cancer, as described in PCT Publication WO 2016/126608 and US Publication No. 2018/0044424. In such embodiments, the conditionally expressed agent is expressed upon activation of the effector cell (e.g., T cell), e.g., the binding of the nonconditional CAR to its target. In one embodiment, the conditionally expressed agent is a CAR (referred to herein as a conditional CAR). In another embodiment, the conditionally expressed agent inhibits a checkpoint inhibitor of the immune response. In another embodiment, the conditionally expressed agent improves or enhances the efficacy of a CAR, and can include a cytokine.

In another embodiment, the therapeutic efficacy of CAR T cells is enhanced by modifying the CAR T cell with a nucleic acid that is capable of altering (e.g., downmodulating) expression of an endogenous gene selected from the group consisting of TCR α chain, TCR β chain, beta-2 microglobulin, a HLA molecule, CTLA-4, PD1, and FAS, as described in PCT Publication WO 2016/069282 and US Publication No. 2017/0335331.

In another embodiment, the therapeutic efficacy of CAR T cells is enhanced by co-expressing in the ‘I’ cells the CAR and one or more enhancers of T cell priming (“ETPs”), as described in PCT Publication WO 2015/112626 and US Publication No. 2016/0340406. The addition of an ETP component to the CAR T cell confers enhanced “professional” antigen-presenting cell (APC) function. In an embodiment, the CAR and one or more ETPs are transiently co-expressed in the T cell. Thus, the engineered T cells are safe (given the transient nature of the CAR/ETP expression), and induce prolonged immunity via APC function.

In another embodiment, the therapeutic efficacy of CAR T cells is enhanced by co-expressing in the T cells a CAR and an inhibitory membrane protein (IMP) comprising a binding (or dimerization) domain, as described in PCT Publication WO 2016/055551 and US Publication No. 2017/0292118. The CAR and the LMP are made both reactive to a soluble compound, especially through a second binding domain comprised within the CAR, thereby allowing the co-localization, by dimerization or ligand recognition, of the inhibitory signaling domain borne by the IMP and of the signal transducing domain borne by the CAR, having the effect of turning down the CAR activation. The inhibitory signaling domain is preferably the programmed death-1 (PD-1), which attenuates T-cell receptor (TCR)-mediated activation of IL-2 production and T-cell proliferation.

In another embodiment, the therapeutic efficacy of CAR T cells is enhanced using a system where controlled variations in the conformation of the extracellular portion of a CAR containing the antigen-binding domain is obtained upon addition of small molecules, as described in PCT Publication WO 2017/032777. This integrated system switches the interaction between the antigen and the antigen binding domain between on/off states. By being able to control the conformation of the extracellular portion of a CAR, downstream functions of the CAR T cell, such as cytotoxicity, can be directly modulated. Thus, a CAR can be characterized in that it comprises: a) at least one ectodomain which comprises: i) an extracellular antigen binding domain; and ii) a switch domain comprising at least a first multimerizing ligand-binding domain and a second multimerizing ligand-binding domain which are capable of binding to a predetermined multivalent ligand to form a multimer comprising said two binding domains and the multivalent ligand to which they are capable of binding; b) at least one transmembrane domain; and c) at least one endodomain comprising a signal transducing domain and optionally a co-stimulatory domain; wherein the switch domain is located between the extracellular antigen binding domain and the transmembrane domain.

Amphiphilic Conjugates

A. Overview

An amphiphile vaccine technology has been developed that involves linking adjuvants or antigens (e.g., peptides) to lipophilic polymeric tails, which promotes localization of vaccines to lymph node (Liu et al. (2014) Nature 507:519-522). Such amphiphile-antigens (e.g., amph-peptides) are also capable of inserting into cell membranes (see e.g., Liu et al. (2011) Angewandte Chemie-Intl. Ed. 50:7052-7055). Accordingly, the present disclosure provides amphiphilic conjugates comprising a CAR ligand for use in stimulating, expanding, activating CAR effector cells (e.g., CAR-T cells).

In some embodiments, the amphiphilic conjugates of the disclosure are used with chimeric antigen receptor (CAR) expressing cell therapy (e.g., CAR-T cell therapy). In some embodiments, the amphiphilic conjugates of the disclosure stimulate a specific immune response against a specific target, such as a tumor-associated antigen. In some embodiments, the amphiphilic conjugates of the disclosure stimulate proliferation of CAR expressing cells (e.g., CAR-T cells) in vivo. In some embodiments, the amphiphilic conjugates of the disclosure comprise a CAR ligand, referred to herein as an amphiphilic ligand conjugate. In some embodiments, the amphiphilic conjugate comprises an immunostimulatory oligonucleotide and is referred to herein as an amphiphilic oligonucleotide conjugate.

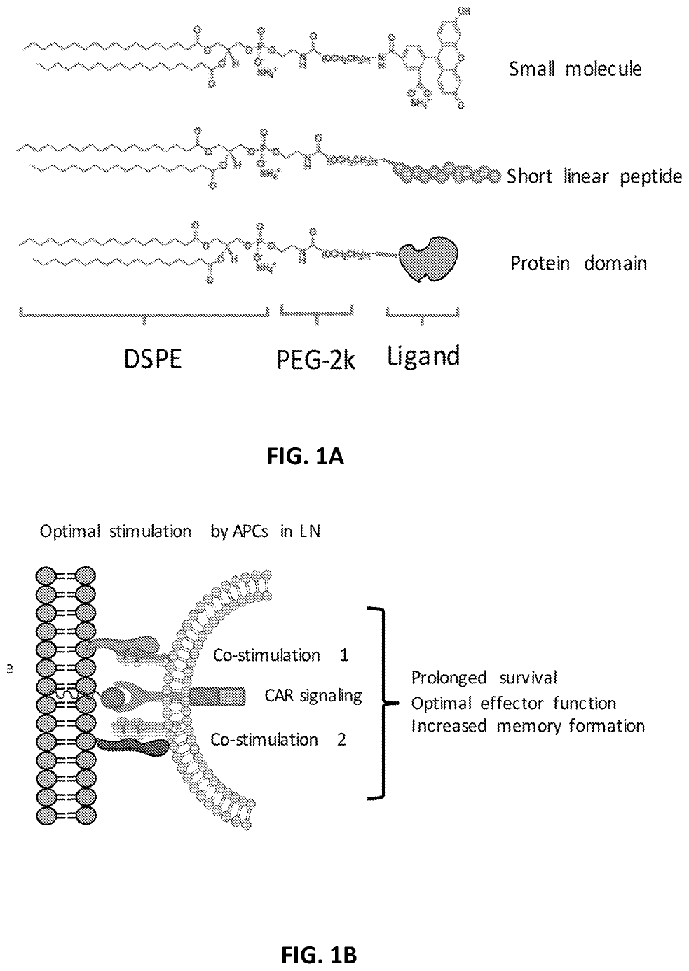

As shown in FIG. 1A, a diversity of amphiphilic ligand conjugate structures are disclosed wherein a lipophilic moiety, or “lipid tail”, (e.g. DSPE) is linked (e.g., covalently linked) via a linker (e.g., PEG-2000), to a CAR ligand. The modularity of this design allows for various ligands including, but not limited to, small molecules (e.g. FITC), short peptides (e.g. a linear peptide providing an epitope specific for CARs), or modular protein domains (e.g. folded polypeptide or polypeptide fragment providing a conformational epitope specific for CARs) to be linked to the lipid (e.g., covalently), resulting in amphiphilic ligand conjugates with tailored specificity.

Without being bound by theory, the amphiphilic ligand conjugate of the disclosure is believed to be delivered primarily to lymph nodes where the lipid tail portion is inserted into the membrane of antigen presenting cells (APCs), resulting in the decoration of the APC with a CAR ligand (FIG. 1B). The embedded CAR ligands function as specific targets for CARs expressed on the surface of CAR expressing cells (e.g., CAR T cells) (which are administered prior to, subsequent or co-administered with the amphiphilic ligand conjugate of the disclosure) resulting in the recruitment of CAR expressing cells to the CAR ligand-decorated APCs. Interaction of the CAR with the embedded CAR-ligand provides a stimulatory signal through the CAR while the APC additionally presents other naturally occurring co-stimulatory signals, resulting in optimal CAR expressing cell activation, prolonged survival and efficient memory formation.

B. Lipid Conjugates

In certain embodiments, a lipid conjugate (e.g., an amphiphilic conjugate), as described in US 2013/0295129, herein incorporated by reference, is used in the methods disclosed herein. In some embodiments, a lipid conjugate comprises a hydrophobic tail that inserts into a cell membrane. In some embodiments, a lipid conjugate comprises an albumin-binding lipid to efficiently target the conjugate to lymph nodes in vivo. In some embodiments, a lipid conjugate comprises an albumin-binding lipid comprising a hydrophobic tail, wherein the hydrophobic tail inserts into the cell membrane, and wherein the conjugate is efficiently targeted to lymph nodes in vivo. In some embodiments, lipid conjugates bind to endogenous albumin, which targets them to lymphatics and draining lymph nodes where they accumulate due to the filtering of albumin by antigen presenting cells. In some embodiments, the lipid conjugate includes an antigenic peptide or molecular adjuvant, and thereby induces or enhances a robust immune response. In some embodiments, the lipid conjugate includes a CAR ligand, and thereby induces or enhances expansion, proliferation, and/or activation of CAR expressing cells (e.g., CAR effector cells, e.g., CAR-T cells). Lipid conjugates comprising a CAR ligand are referred to as “amphiphilic ligand conjugates” as defined supra.

In some embodiments, the lipid conjugates efficiently targeted to the lymph nodes are referred to as “lymph node-targeting conjugates.” In some embodiments, lymph node-targeting conjugates comprises a highly lipophilic, albumin-binding domain (e.g., an albumin-binding lipid), and a cargo such as a CAR ligand or molecular adjuvant. In some embodiments, lymph node-targeting conjugates include three domains: a highly lipophilic, albumin-binding domain (e.g., an albumin-binding lipid), a cargo such as a CAR ligand or molecular adjuvant, and a polar block linker, which promotes solubility of the conjugate and reduces the ability of the lipid to insert into cellular plasma membranes. Accordingly, in certain embodiments, the general structure of the conjugate is L-P-C, where “L” is an albumin-binding lipid, “P” is a polar block, and “C” is a cargo such as a CAR ligand or a molecular adjuvant. In some embodiments, the cargo itself can also serve as the polar block domain, and a separate polar block domain is not required. Therefore, in certain embodiments the conjugate has only two domains: an albumin-binding lipid and a cargo.

In some embodiments, the cargo of the conjugate is a CAR ligand, thereby resulting in an amphiphilic ligand conjugate. In some embodiments, the amphiphilic ligand conjugate is administered or formulated with an adjuvant, wherein the adjuvant is an amphiphilic ligand comprising a molecular adjuvant such as an immunostimulatory oligonucleotide, or a peptide antigen, as the cargo.

(i) Lipids

In some embodiments, the lipid component of the amphiphilic conjugates comprises a hydrophobic tail. In some embodiments, the hydrophobic tail inserts into a cell membrane. In some embodiments, the lipid is linear, branched, or cyclic. In some embodiments, the lipid is greater than 12 carbons in length. In some embodiments, the lipid is 13 carbons in length. In some embodiments, the lipid is 14 carbons in length. In some embodiments, the lipid is 15 carbons in length. In some embodiments, the lipid is 16 carbons in length. In some embodiments, the lipid is 17 carbons in length. In some embodiments, the lipid is 18 carbons in length. In some embodiments, the lipid is 19 carbons in length. In some embodiments, the lipid is 20 carbons in length. In some embodiments, the lipid is 21 carbons in length. In some embodiments, the lipid is 22 carbons in length. In some embodiments, the lipid is 23 carbons in length. In some embodiments, the lipid is 24 carbons in length. In some embodiments, the lipid is 25 carbons in length. In some embodiments, the lipid is 26 carbons in length. In some embodiments, the lipid is 27 carbons in length. In some embodiments, the lipid is 28 carbons in length. In some embodiments, the lipid is 29 carbons in length. In some embodiments, the lipid is 30 carbons in length. In some embodiments, the lipid at least 17 to 18 carbons in length, but may be shorter if it shows good albumin binding and adequate targeting to the lymph nodes.

Lymph node-targeting conjugates include amphiphilic ligand conjugates and amphiphilic oligonucleotide conjugates that can be trafficked from the site of delivery through the lymph to the lymph node. In certain embodiments, the activity relies, in-part, on the ability of the conjugate to associate with albumin in the blood of the subject. Therefore, lymph node-targeted conjugates typically include a lipid that can bind to albumin under physiological conditions. Lipids suitable for targeting the lymph node can be selected based on the ability of the lipid or a lipid conjugate including the lipid to bind to albumin. Suitable methods for testing the ability of the lipid or lipid conjugate to bind to albumin are known in the art.

For example, in certain embodiments, a plurality of lipid conjugates is allowed to spontaneously form micelles in aqueous solution. The micelles are incubated with albumin, or a solution including albumin such as Fetal Bovine Serum (FBS). Samples can be analyzed, for example, by ELISA, size exclusion chromatography or other methods to determine if binding has occurred. Lipid conjugates can be selected as lymph node-targeting conjugates if in the presence of albumin, or a solution including albumin such as Fetal Bovine Serum (FBS), the micelles dissociate and the lipid conjugates bind to albumin as discussed above.

Examples of preferred lipids for use in lymph node targeting lipid conjugates include, but are not limited to, fatty acids with aliphatic tails of 8-30 carbons including, but not limited to, linear unsaturated and saturated fatty acids, branched saturated and unsaturated fatty acids, and fatty acids derivatives, such as fatty acid esters, fatty acid amides, and fatty acid thioesters, diacyl lipids, cholesterol, cholesterol derivatives, and steroid acids such as bile acids, Lipid A or combinations thereof. In some embodiments, the lipid is saturated. In some embodiments, the lipid comprises at least one lipid tail comprising 8-30, 12-30, 15-25, or 16-20 carbons.

In certain embodiments, the lipid is a diacyl lipid or two-tailed lipid. In some embodiments, the tails in the diacyl lipid contain from about 8 to about 30 carbons and can be saturated, unsaturated, or combinations thereof. In some embodiments, the diacyl lipid is saturated. In some embodiments, the diacyl lipid is saturated and each tail comprises about 8 to about 30 carbons. In some embodiments, the diacyl lipid is saturated and each tail comprises 12 carbons. In some embodiments, the diacyl lipid is saturated and each tail comprises 13 carbons. In some embodiments, the diacyl lipid is saturated and each tail comprises 14 carbons. In some embodiments, the diacyl lipid is saturated and each tail comprises 15 carbons. In some embodiments, the diacyl lipid is saturated and each tail comprises 16 carbons. In some embodiments, the diacyl lipid is saturated and each tail comprises 17 carbons. In some embodiments, the diacyl lipid is saturated and each tail comprises 18 carbons. In some embodiments, the diacyl lipid is saturated and each tail comprises 19 carbons. In some embodiments, the diacyl lipid is saturated and each tail comprises 20 carbons. In some embodiments, the diacyl lipid is saturated and each tail comprises 21 carbons. In some embodiments, the diacyl lipid is saturated and each tail comprises 22 carbons. In some embodiments, the diacyl lipid is saturated and each tail comprises 23 carbons. In some embodiments, the diacyl lipid is saturated and each tail comprises 24 carbons. In some embodiments, the diacyl lipid is saturated and each tail comprises 25 carbons. In some embodiments, the diacyl lipid is saturated and each tail comprises 26 carbons. In some embodiments, the diacyl lipid is saturated and each tail comprises 27 carbons. In some embodiments, the diacyl lipid is saturated and each tail comprises 28 carbons. In some embodiments, the diacyl lipid is saturated and each tail comprises 29 carbons. In some embodiments, the diacyl lipid is saturated and each tail comprises 30 carbons. The tails can be coupled to the head group via ester bond linkages, amide bond linkages, thioester bond linkages, or combinations thereof. In a particular embodiment, the diacyl lipids are phosphate lipids, glycolipids, sphingolipids, or combinations thereof.

In some embodiments, the lipid is 1,2-distearoyl-sn-glycero-3-phosphoethanolamine (DSPE). In some embodiments, a diacyl lipid is synthesized as described in U.S. Pat. No. 9,107,904, herein incorporated by reference in its entirety. In some embodiments, a diacyl lipid is synthesized as provided below:

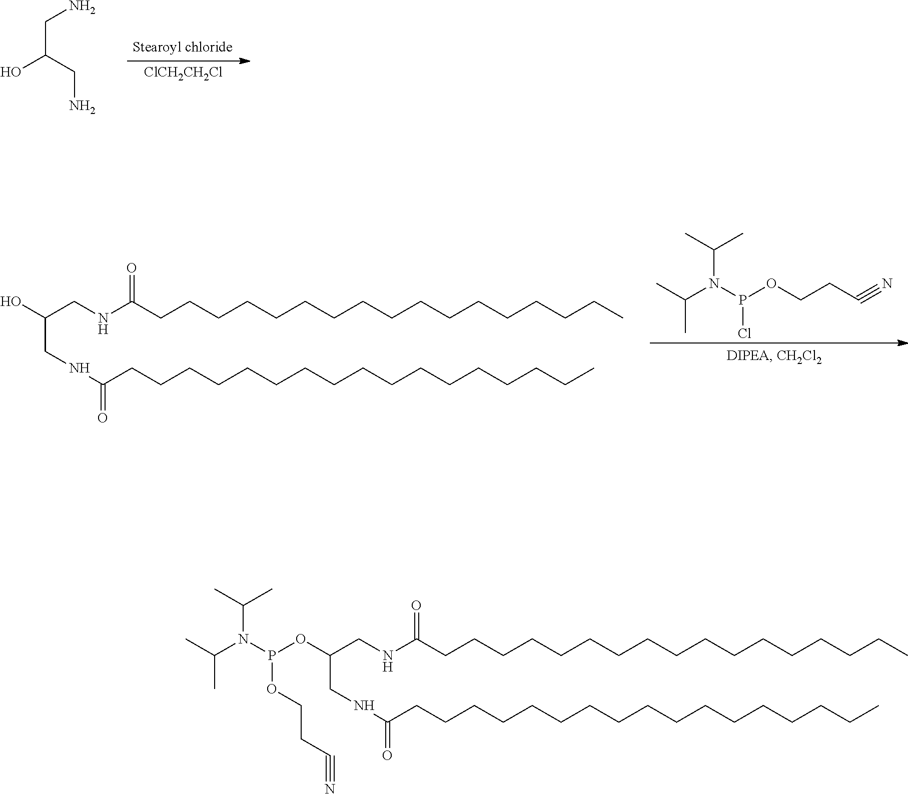

Preferably, lymph node-targeting conjugates include a lipid that is 8 or more carbon units in length. It is believed that increasing the number of lipid units can reduce insertion of the lipid into plasma membrane of cells, allowing the lipid conjugate to remain free to bind albumin and traffic to the lymph node.

For example, in some embodiments, the lipid can be a diacyl lipid composed of two C18 hydrocarbon tails. In certain embodiments, the lipid for use in preparing lymph node targeting lipid conjugates is not a single chain hydrocarbon (e.g., C18).

(ii) Molecular Adjuvants

In certain embodiments, amphiphilic oligonucleotide conjugates are used with the amphiphilic ligand conjugate. The oligonucleotide conjugates typically contain an immunostimulatory oligonucleotide.

In certain embodiments, the immunostimulatory oligonucleotide can serve as a ligand for pattern recognition receptors (PRRs). Examples of PRRs include the Toll-like family of signaling molecules that play a role in the initiation of innate immune responses and also influence the later and more antigen specific adaptive immune responses. Therefore, the oligonucleotide can serve as a ligand for a Toll-like family signaling molecule, such as Toll-Like Receptor 9 (TLR9).

For example, unmethylated CpG sites can be detected by TLR9 on plasmacytoid dendritic cells and B cells in humans (Zaida, et al., Infection and Immunity, 76(5):2123-2129, (2008)). Therefore, the sequence of oligonucleotide can include one or more unmethylated cytosine-guanine (CG or CpG, used interchangeably) dinucleotide motifs. The ‘p’ refers to the phosphodiester backbone of DNA, as discussed in more detail below, some oligonucleotides including CG can have a modified backbone, for example a phosphorothioate (PS) backbone. In certain embodiments, an immunostimulatory oligonucleotide can contain more than one CG dinucleotide, arranged either contiguously or separated by intervening nucleotide(s). The CpG motif(s) can be in the interior of the oligonucleotide sequence. Numerous nucleotide sequences stimulate TLR9 with variations in the number and location of CG dinucleotide(s), as well as the precise base sequences flanking the CG dimers.

Typically, CG ODNs are classified based on their sequence, secondary structures, and effect on human peripheral blood mononuclear cells (PBMCs). The five classes are Class A (Type D), Class B (Type K), Class C, Class P, and Class S (Vollmer, J & Krieg, A M, Advanced drug delivery reviews 61(3): 195-204 (2009), incorporated herein by reference). CG ODNs can stimulate the production of Type I interferons (e.g., IFNα) and induce the maturation of dendritic cells (DCs). Some classes of ODNs are also strong activators of natural killer (NK) cells through indirect cytokine signaling. Some classes are strong stimulators of human B cell and monocyte maturation (Weiner, G L, PNAS USA 94(20): 10833-7 (1997); Dalpke, A H, Immunology 106(1): 102-12 (2002); Hartmann, G, J of Immun. 164(3):1617-2 (2000), each of which is incorporated herein by reference).

According to some embodiments, a lipophilic-CpG oligonucleotide conjugate is used to enhance an immune response to an antigen. The lipophilic-CpG oligonucleotide is represented by the following, wherein “L” is a lipophilic compound, such as diacyl lipid, “Gₙ” is a guanine repeat linker and “n” represents 1, 2, 3, 4, or 5.

5′-L-GₙTCCATGACGTTCCTGACGTT-3′

Other PRR Toll-like receptors include TLR3, and TLR7 which may recognize double-stranded RNA, single-stranded and short double-stranded RNAs, respectively, and retinoic acid-inducible gene I (RIG-I)-like receptors, namely RIG-I and melanoma differentiation-associated gene 5 (MDA5), which are best known as RNA-sensing receptors in the cytosol. Therefore, in certain embodiments, the oligonucleotide contains a functional ligand for TLR3, TLR7, or RIG-I-like receptors, or combinations thereof.

Examples of immunostimulatory oligonucleotides, and methods of making them are known in the art, see for example, Bodera, P. Recent Pat Inflamm Allergy Drug Discov. 5(1):87-93 (2011), incorporated herein by reference.

In certain embodiments, the oligonucleotide cargo includes two or more immunostimulatory sequences.

The oligonucleotide can be between 2-100 nucleotide bases in length, including for example, 5 nucleotide bases in length, 10 nucleotide bases in length, 15 nucleotide bases in length, 20 nucleotide bases in length, 25 nucleotide bases in length, 30 nucleotide bases in length, 35 nucleotide bases in length, 40 nucleotide bases in length, 45 nucleotide bases in length, 50 nucleotide bases in length, 60 nucleotide bases in length, 70 nucleotide bases in length, 80 nucleotide bases in length, 90 nucleotide bases in length, 95 nucleotide bases in length, 98 nucleotide bases in length, 100 nucleotide bases in length or more.

The 3′ end or the 5′ end of the oligonucleotides can be conjugated to the polar block or the lipid. In certain embodiments the 5′ end of the oligonucleotide is linked to the polar block or the lipid.

The oligonucleotides can be DNA or RNA nucleotides which typically include a heterocyclic base (nucleic acid base), a sugar moiety attached to the heterocyclic base, and a phosphate moiety which esterifies a hydroxyl function of the sugar moiety. The principal naturally-occurring nucleotides comprise uracil, thymine, cytosine, adenine and guanine as the heterocyclic bases, and ribose or deoxyribose sugar linked by phosphodiester bonds. In certain embodiments, the oligonucleotides are composed of nucleotide analogs that have been chemically modified to improve stability, half-life, or specificity or affinity for a target receptor, relative to a DNA or RNA counterpart. The chemical modifications include chemical modification of nucleobases, sugar moieties, nucleotide linkages, or combinations thereof. As used herein ‘modified nucleotide” or “chemically modified nucleotide” defines a nucleotide that has a chemical modification of one or more of the heterocyclic base, sugar moiety or phosphate moiety constituents. In certain embodiments, the charge of the modified nucleotide is reduced compared to DNA or RNA oligonucleotides of the same nucleobase sequence. For example, the oligonucleotide can have low negative charge, no charge, or positive charge.

Typically, nucleoside analogs support bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide (e.g., single-stranded RNA or single-stranded DNA). In certain embodiments, the analogs have a substantially uncharged, phosphorus containing backbone.

(iii) Chimeric Antigen Receptor Ligand

In some embodiments, the CAR ligand of the amphiphilic ligand conjugate is an antigenic protein or polypeptide, such as a tumor-associated antigen or portion thereof. In some embodiments, the CAR ligand is a small molecule, peptide or protein domain, or fragment thereof. In some embodiments, the ligand binds to the CAR on CAR expressing cells (e.g., CAR-T cells). Accordingly, the methods and compositions described herein utilize an amphiphilic ligand conjugate complementary to a CAR expressing cell (e.g., CAR-T cell). In some embodiments, the CAR ligand binds to any one of the CARs described supra.

In some embodiments, the peptide is 2-100 amino acids, including for example, 5 amino acids, 10 amino acids, 15 amino acids, 20 amino acids, 25 amino acids, 30 amino acids, 35 amino acids, 40 amino acids, 45 amino acids, or 50 amino acids. In some embodiments, a peptide is greater than 50 amino acids. In some embodiments, the peptide is >100 amino acids. In some embodiments, a protein/peptide is linear, branched or cyclic. In some embodiments, the peptide includes D amino acids, L amino acids, or a combination thereof. In some embodiments, the peptide or protein is conjugated to the polar block or lipid at the N-terminus or the C-terminus of the peptide or protein.

In some embodiments, the protein or polypeptide can be any protein or peptide that can induce or increase the ability of the immune system to develop antibodies and T-cell responses to the protein or peptide. A cancer antigen is an antigen that is typically expressed preferentially by cancer cells (i.e., it is expressed at higher levels in cancer cells than on non-cancer cells) and in some instances it is expressed solely by cancer cells. The cancer antigen may be expressed within a cancer cell or on the surface of the cancer cell. The cancer antigen can be, but is not limited to, CD19, TRP-1, TRP-2, MART-1/Melan-A, gp100, adenosine deaminase-binding protein (ADAbp), FAP, cyclophilin b, colorectal associated antigen (CRC)-0017-1A/GA733, carcinoembryonic antigen (CEA), CAP-1, CAP-2, etv6, AML1, prostate specific antigen (PSA), PSA-1, PSA-2, PSA-3, prostate-specific membrane antigen (PSMA), T cell receptor/CD3-zeta chain, and CD20. The cancer antigen may be selected from the group consisting of MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-05), GAGE-1, GAGE-2, GAGE-3, GAGE-4, GAGE-5, GAGE-6, GAGE-7, GAGE-8, GAGE-9, BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin, γ-catenin, p120ctn, gp100Pmel117, PRAME, NY-ESO-1, cdc27, adenomatous polyposis coli protein (APC), fodrin, Connexin 37, Ig-idiotype, p15, gp75, GM2 ganglioside, GD2 ganglioside, human papilloma virus proteins, Smad family of tumor antigens, lmp-1, PIA, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, CD20, or c-erbB-2.

In some embodiments, the methods and compositions of the disclosure are used in combination with Kymriah(™) (tisagenlecleucel; Novartis) suspension for intravenous infusion, formerly CTL019. For example, in one embodiment, a composition of the disclosure comprises an amphiphilic ligand conjugate in which the CAR ligand is CD19, or an antigenic portion thereof. Such compositions can be administered to subjects in combination with a CD19-specific CAR-T cell (e.g., a population of CD19-specific CAR-T cells), such as Kymriah(™) (tisagenlecleucel; Novartis), for treatment of cancer, for example, B-cell acute lymphoblastic leukemia (ALL).

Suitable antigens are known in the art and are available from commercial government and scientific sources. In certain embodiments, the antigens are whole inactivated or irradiated tumor cells. The antigens may be purified or partially purified polypeptides derived from tumors. The antigens can be recombinant polypeptides produced by expressing DNA encoding the polypeptide antigen in a heterologous expression system. The antigens can be DNA encoding all or part of an antigenic protein. The DNA may be in the form of vector DNA such as plasmid DNA.

In certain embodiments, antigens may be provided as single antigens or may be provided in combination. Antigens may also be provided as complex mixtures of polypeptides or nucleic acids.

In some embodiments, the CAR ligand of the amphiphilic ligand conjugate is a tag, which binds to a CAR comprising a tag binding domain, as described supra. In some embodiments, the tag is fluorescein isothiocyanate (FITC), streptavidin, biotin, dinitrophenol, peridinin chlorophyll protein complex, green fluorescent protein, phycoerythrin (PE), horse radish peroxidase, palmitoylation, nitrosylation, alkalanine phosphatase, glucose oxidase, or maltose binding protein.

In some embodiments, the CAR comprises a tumor antigen binding domain, and the CAR ligand is the tumor antigen or a fragment thereof. In some embodiments, the CAR comprises a tag binding domain (e.g., AT-CAR), and the CAR ligand is the tag. In some embodiments, the CAR is a tandem CAR, and the CAR ligand binds to at least one of the antigen binding domains present on the tandem CAR. In some embodiments, the CAR is a bispecific and comprises a tumor antigen binding domain and a tag binding domain, and the CAR ligand is the tag. In some embodiments, the CAR is a bispecific and comprises a tumor antigen binding domain and a tag binding domain, and the CAR ligand is the tumor antigen or fragment thereof. In some embodiments, the CAR comprises a first tumor-associated antigen binding domain and a second tumor-associated antigen binding domain, and the CAR ligand is the first or second tumor-associated antigen.

(iv) Polar Block/Linker

For the conjugate to be trafficked efficiently to the lymph node, the conjugate should remain soluble. Therefore, in some embodiments a polar block linker is included between the cargo and the lipid to increase solubility of the conjugate. The polar block reduces or prevents the ability of the lipid to insert into the plasma membrane of cells, such as cells in the tissue adjacent to the injection site. The polar block can also reduce or prevent the ability of cargo, such as synthetic oligonucleotides containing a PS backbone, from non-specifically associating with extracellular matrix proteins at the site of administration. In some embodiments, the polar block increases the solubility of the conjugate without preventing its ability to bind to albumin. It is believed that this combination of characteristics allows the conjugate to bind to albumin present in the serum or interstitial fluid, and remain in circulation until the albumin is trafficked to, and retained in a lymph node. In some embodiments, the cargo functions as the polar block, and therefore a separate polar block is not required.

The length and composition of the polar block can be adjusted based on the lipid and cargo selected. For example, for oligonucleotide conjugates, the oligonucleotide itself may be polar enough to insure solubility of the conjugate, for example, oligonucleotides that are 10, 15, 20 or more nucleotides in length. Therefore, in certain embodiments, no additional polar block linker is required. However, depending on the amino acid sequence, some lipidated peptides can be essentially insoluble. In these cases, it can be desirable to include a polar block that mimics the effect of a polar oligonucleotide.

In some embodiments, a polar block is used as part of any of the lipid conjugates suitable for use in the methods disclosed herein, for example, amphiphilic oligonucleotide conjugates and amphiphilic ligand conjugates, which reduce cell membrane insertion/preferential portioning on albumin. In some embodiments, suitable polar blocks include, but are not limited to, oligonucleotides such as those discussed above, a hydrophilic polymer including but not limited to poly(ethylene glycol) (MW: 500 Da to 20,000 Da), polyacrylamide (MW: 500 Da to 20,000 Da), polyacrylic acid; a string of hydrophilic amino acids such as serine, threonine, cysteine, tyrosine, asparagine, glutamine, aspartic acid, glutamic acid, lysine, arginine, histidine, or combinations thereof polysaccharides, including but not limited to, dextran (MW: 1,000 Da to 2,000,000 Da), or combinations thereof.

In some embodiments, the polar block, whether a separate component or the cargo itself, provides solubility to the overall lipid conjugate based on the molecular weight of the polar block. For example, in some embodiments, a polar block having a molecular weight of 2,000 Da is sufficient to make the lipid conjugate soluble for albumin binding. In some embodiments, the polar block has a molecular weight of about 300 to about 20,000 Da. In some embodiments, the polar block has a molecular weight of about 1,000 to about 15,000 Da. In some embodiments, the polar block has a molecular weight of about 1,500 to about 10,000 Da. In some embodiments, the polar block has a molecular weight of about 2,000 to about 5,000 Da. In some embodiments, the polar block has a molecular weight of about 1,000 to about 2,500 Da. In some embodiments, the polar block has a molecular weight of about 1,000 to about 3,000 Da. In some embodiments, the polar block has a molecular weight of about 1,000 to about 3,500 Da. In some embodiments, the polar block has a molecular weight of about 1,000 to about 4,000 Da. In some embodiments, the polar block has a molecular weight of about 1,000 to about 5,000 Da. In some embodiments, the polar block has a molecular weight of about 5,000 to about 10,000 Da. In some embodiments, the polar block has a molecular weight of about 15,000 to about 20,000 Da.

In some embodiments, the hydrophobic lipid and the linker/cargo are covalently linked. In some embodiments, the covalent bond is a non-cleavable linkage or a cleavable linkage. In some embodiments, the non-cleavable linkage includes an amide bond or phosphate bond, and the cleavable linkage includes a disulfide bond, acid-cleavable linkage, ester bond, anhydride bond, biodegradable bond, or enzyme-cleavable linkage.

a. Ethylene Glycol Linkers

In certain embodiments, the polar block is one or more ethylene glycol (EG) units, more preferably two or more EG units (i.e., polyethylene glycol (PEG)). For example, in certain embodiments, a lipid conjugate includes a cargo (i.e., CAR ligand or molecular adjuvant) and a hydrophobic lipid linked by a polyethylene glycol (PEG) molecule or a derivative or analog thereof.

In certain embodiments, lipid conjugates suitable for use in the methods disclosed herein contain a CAR ligand linked to PEG which is in turn linked to a hydrophobic lipid, or lipid-Gn-ON conjugates, either covalently or via formation of protein-oligo conjugates that hybridize to oligo micelles. The precise number of EG units depends on the lipid and the cargo, however, typically, a polar block can have between about 1 and about 100, between about 20 and about 80, between about 30 and about 70, or between about 40 and about 60 EG units. In certain embodiments, the polar block has between about 45 and 55 EG, units. For example, in certain embodiments, the polar block has 48 EG units.

In some embodiments, the PEG molecule has a molecular weight of about 300-20,000 daltons. In some embodiments, the PEG molecule has a molecular weight of about 1,000 daltons. In some embodiments, the PEG molecule has a molecular weight of about 1,500 daltons. In some embodiments, the PEG molecule has a molecular weight of about 2,000 daltons. In some embodiments, the PEG molecule has a molecular weight of about 2,500 daltons. In some embodiments, the PEG molecule has a molecular weight of about 3,000 daltons. In some embodiments, the PEG molecule has a molecular weight of about 3,500 daltons. In some embodiments, the PEG molecule has a molecular weight of about 4,000 daltons. In some embodiments, the PEG molecule has a molecular weight of about 5,000 daltons. In some embodiments, the PEG molecule has a molecular weight of about 6,000 daltons. In some embodiments, the PEG molecule has a molecular weight of about 7,000 daltons. In some embodiments, the PEG molecule has a molecular weight of about 8,000 daltons. In some embodiments, the PEG molecule has a molecular weight of about 9,000 daltons. In some embodiments, the PEG molecule has a molecular weight of about 10,000 daltons. In some embodiments, the PEG molecule has a molecular weight of about 11,000 daltons. In some embodiments, the PEG molecule has a molecular weight of about 12,000 daltons. In some embodiments, the PEG molecule has a molecular weight of about 13,000 daltons. In some embodiments, the PEG molecule has a molecular weight of about 14,000 daltons. In some embodiments, the PEG molecule has a molecular weight of about 15,000 daltons. In some embodiments, the PEG molecule has a molecular weight of about 16,000 daltons. In some embodiments, the PEG molecule has a molecular weight of about 17,000 daltons. In some embodiments, the PEG molecule has a molecular weight of about 18,000 daltons. In some embodiments, the PEG molecule has a molecular weight of about 19,000 daltons. In some embodiments, the PEG molecule has a molecular weight of about 20,000 daltons.

b. Oligonucleotide Linkers

As discussed above, in certain embodiments, the polar block is an oligonucleotide. The polar block linker can have any sequence, for example, the sequence of the oligonucleotide can be a random sequence, or a sequence specifically chosen for its molecular or biochemical properties (e.g., highly polar). In certain embodiments, the polar block linker includes one or more series of consecutive adenine (A), cytosine (C), guanine (G), thymine (T), uracil (U), or analog thereof. In certain embodiments, the polar block linker consists of a series of consecutive adenine (A), cytosine (C), guanine (G), thymine (T), uracil (U), or analog thereof.

In certain embodiments, the linker is one or more guanines, for example between 1-10 guanines. It has been discovered that altering the number of guanines between a cargo such as a CpG oligonucleotide, and a lipid tail controls micelle stability in the presence of serum proteins. Therefore, the number of guanines in the linker can be selected based on the desired affinity of the conjugate for serum proteins such as albumin. When the cargo is a CpG immunostimulatory oligonucleotide and the lipid tail is a diacyl lipid, the number of guanines affects the ability of micelles formed in aqueous solution to dissociate in the presence of serum: 20% of the non-stabilized micelles (lipo-G₂T₁₀-CG) were intact, while the remaining 80% were disrupted and bonded with FBS components. In the presence of guanines, the percentage of intact micelles increased from 36% (lipo-G₂T₈-CG) to 73% (lipo-G₄T₆-CG), and finally reached 90% (lipo-G₆T₄-CG). Increasing the number of guanines to eight (lipo-G₈T₂-CG) and ten (lipo-G₁₀T₀-CG) did not further enhance micelle stability.

Therefore, in certain embodiments, the linker in a lymph node-targeting conjugate suitable for use in the methods disclosed herein can include 0, 1, or 2 guanines. As discussed in more detail below, linkers that include 3 or more consecutive guanines can be used to form micelle-stabilizing conjugates with properties that are suitable for use in the methods disclosed herein.

C. Immunogenic Compositions

The lipid conjugates suitable for use in the methods disclosed herein can be used in immunogenic compositions or as components in vaccines. Typically, immunogenic compositions disclosed herein include an adjuvant, an antigen, or a combination thereof. The combination of an adjuvant and an antigen can be referred to as a vaccine. When administered to a subject in combination, the adjuvant and antigen can be administered in separate pharmaceutical compositions, or they can be administered together in the same pharmaceutical composition. When administered in combination, the adjuvant can be a lipid conjugate, the antigen can be a lipid conjugate, or the adjuvant and the antigen can both be lipid conjugates.

In some embodiments, an immunogenic composition suitable for use in the methods disclosed herein includes an amphiphilic ligand conjugate administered alone, or in combination with an adjuvant. In some embodiments, the adjuvant is without limitation alum (e.g., aluminum hydroxide, aluminum phosphate); saponins purified from the bark of the Q. saponaria tree such as QS21 (a glycolipid that elutes in the 21st peak with HPLC fractionation; Antigenics, Inc., Worcester, Mass.); poly[di(carboxylatophenoxy)phosphazene (PCPP polymer; Virus Research Institute, USA), Flt3 ligand, Leishmania elongation factor (a purified Leishmania protein; Corixa Corporation, Seattle, Wash.), ISCOMS (immunostimulating complexes which contain mixed saponins, lipids and form virus-sized particles with pores that can hold antigen; CSL, Melbourne, Australia), Pam3Cys, SB-AS4 (SmithKline Beecham adjuvant system #4 which contains alum and MPL; SBB, Belgium), non-ionic block copolymers that form micelles such as CRL 1005 (these contain a linear chain of hydrophobic polyoxypropylene flanked by chains of polyoxyethylene, Vaxcel, Inc., Norcross, Ga.), and Montanide IMS (e.g., IMS 1312, water-based nanoparticles combined with a soluble immunostimulant, Seppic).

In some embodiments, an adjuvant is a TLR ligand, such as those discussed above. In some embodiments, adjuvants that act through TLR3 include, without limitation, double-stranded RNA. In some embodiments, adjuvants that act through TLR4 include, without limitation, derivatives of lipopolysaccharides such as monophosphoryl lipid A (MPLA; Ribi ImmunoChem Research, Inc., Hamilton, Mont.) and muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland). In some embodiments, adjuvants that act through TLR5 include, without limitation, flagellin. In some embodiments, adjuvants that act through TLR7 and/or TLR8 include single-stranded RNA, oligoribonucleotides (ORN), synthetic low molecular weight compounds such as imidazoquinolinamines (e.g., imiquimod (R-837), resiquimod (R-848)). In some embodiments, adjuvants acting through TLR9 include DNA of viral or bacterial origin, or synthetic oligodeoxynucleotides (ODN), such as CpG ODN. In some embodiments, another adjuvant class is phosphorothioate containing molecules such as phosphorothioate nucleotide analogs and nucleic acids containing phosphorothioate backbone linkages.

In some embodiments, the adjuvant is selected from oil emulsions (e.g., Freund's adjuvant); saponin formulations; virosomes and viral-like particles; bacterial and microbial derivatives; immunostimulatory oligonucleotides; ADP-ribosylating toxins and detoxified derivatives; alum; BCG; mineral-containing compositions (e.g., mineral salts, such as aluminum salts and calcium salts, hydroxides, phosphates, sulfates, etc.); bioadhesives and/or mucoadhesives; microparticles; liposomes; polyoxyethylene ether and polyoxyethylene ester formulations; polyphosphazene; muramyl peptides; imidazoquinolone compounds; and surface active substances (e.g. lysolecithin, pluronic polyols, polyanions, peptides, oil emulsions, keyhole limpet hemocyanin, and dinitrophenol).

In some embodiments, an adjuvant is selected from immunomodulators such as cytokines, interleukins (e.g., IL-1, IL-2, IL-4, IL-5, IL-6, IL-7, IL-12, etc.), interferons (e.g., interferon-.gamma.), macrophage colony stimulating factor, and tumor necrosis factor.

In some embodiments, the adjuvant is an amphiphilic oligonucleotide conjugate comprising an immunostimulatory oligonucleotide, as described supra.

In some embodiments, the adjuvant is a STING (STimulator of Interferon Genes) agonist. The STING signaling pathway in immune cells is a central mediator of innate immune response and when stimulated, induces expression of various interferons, cytokines and T cell recruitment factors that amplify and strengthen immune activity. Recent work has shown that STING agonists are effective adjuvants and efficiently elicit an immune response, described, for example in Dubensky, T., et al., Therapeutic Advances in Vaccines, Vol. 1(4): 131-143 (2013); and Hanson, M., et al., The Journal of Clinical Investigation, Vol. 125 (6): 2532-2546 (2015), hereby incorporated by reference.

In some embodiments, a STING agonist is a cyclic dinucleotide. In certain embodiments, cyclic dinucleotides include, but are not limited to, cdAMP, cdGMP, cdIMP, c-AMP-GMP, c-AMP-IMP, and c-GMP-IMP, and analogs thereof including, but not limited to, phosphorothioate analogues. In some embodiments, suitable cyclic dinucleotides for use in the present disclosure are described in some detail in, U.S. Pat. Nos. 7,709,458 and 7,592,326; WO 2007/054779; US 2014/0205653; and Yan et al. Bioorg. Med. Chem Lett. 18: 5631 (2008) each of which is hereby incorporated by reference.

In certain embodiments, a STING agonist is chemically synthesized. In certain embodiments, a STING agonist is an analog of a naturally occurring cyclic dinucleotide. STING agonists, including analogs of cyclic dinucleotides, suitable for use in the disclosure are provided in U.S. Pat. Nos. 7,709,458 and 7,592,326; and US 2014/0205653.

Methods of Making Polypeptides

In some embodiments, the polypeptides described herein for use in the amphiphilic conjugates (e.g., tumor associated antigens) are made in transformed host cells using recombinant DNA techniques. To do so, a recombinant DNA molecule coding for the peptide is prepared. Methods of preparing such DNA molecules are well known in the art. For instance, sequences coding for the peptides could be excised from DNA using suitable restriction enzymes. Alternatively, the DNA molecule could be synthesized using chemical synthesis techniques, such as the phosphoramidate method. Also, a combination of these techniques could be used.

The methods of making polypeptides also include a vector capable of expressing the peptides in an appropriate host. The vector comprises the DNA molecule that codes for the peptides operatively linked to appropriate expression control sequences. Methods of affecting this operative linking, either before or after the DNA molecule is inserted into the vector, are well known. Expression control sequences include promoters, activators, enhancers, operators, ribosomal nuclease domains, start signals, stop signals, cap signals, polyadenylation signals, and other signals involved with the control of transcription or translation.

The resulting vector having the DNA molecule thereon is used to transform an appropriate host. This transformation may be performed using methods well known in the art.

Any of a large number of available and well-known host cells may be suitable for use in the methods disclosed herein. The selection of a particular host is dependent upon a number of factors recognized by the art. These include, for example, compatibility with the chosen expression vector, toxicity of the peptides encoded by the DNA molecule, rate of transformation, ease of recovery of the peptides, expression characteristics, bio-safety and costs. A balance of these factors must be struck with the understanding that not all hosts may be equally effective for the expression of a particular DNA sequence. Within these general guidelines, useful microbial hosts include bacteria (such as E. coli sp.), yeast (such as Saccharomyces sp.) and other fungi, insects, plants, mammalian (including human) cells in culture, or other hosts known in the art.

Next, the transformed host is cultured and purified. Host cells may be cultured under conventional fermentation conditions so that the desired compounds are expressed. Such fermentation conditions are well known in the art. Finally, the peptides are purified from culture by methods well known in the art.

The compounds may also be made by synthetic methods. For example, solid phase synthesis techniques may be used. Suitable techniques are well known in the art, and include those described in Merrifield (1973), Chem. Polypeptides, pp. 335-61 (Katsoyannis and Panayotis eds.); Merrifield (1963), J. Am. Chem. Soc. 85: 2149; Davis et al. (1985), Biochem. Intl. 10: 394-414; Stewart and Young (1969), Solid Phase Peptide Synthesis; U.S. Pat. No. 3,941,763; Finn et al. (1976), The Proteins (3rd ed.) 2: 105-253; and Erickson et al. (1976), The Proteins (3rd ed.) 2: 257-527. Solid phase synthesis is the preferred technique of making individual peptides since it is the most cost-effective method of making small peptides. Compounds that contain derivatized peptides or which contain non-peptide groups may be synthesized by well-known organic chemistry techniques.

Other methods are of molecule expression/synthesis are generally known in the art to one of ordinary skill.

The nucleic acid molecules described above can be contained within a vector that is capable of directing their expression in, for example, a cell that has been transduced with the vector. Accordingly, in addition to polypeptide mutants, expression vectors containing a nucleic acid molecule encoding a mutant and cells transfected with these vectors are among the certain embodiments.

Vectors suitable for use include T7-based vectors for use in bacteria (see, for example, Rosenberg et al., Gene 56: 125, 1987), the pMSXND expression vector for use in mammalian cells (Lee and Nathans, J. Biol. Chem. 263:3521, 1988), and baculovirus-derived vectors (for example the expression vector pBacPAKS from Clontech, Palo Alto, Calif.) for use in insect cells. The nucleic acid inserts, which encode the polypeptide of interest in such vectors, can be operably linked to a promoter, which is selected based on, for example, the cell type in which expression is sought. For example, a T7 promoter can be used in bacteria, a polyhedrin promoter can be used in insect cells, and a cytomegalovirus or metallothionein promoter can be used in mammalian cells. Also, in the case of higher eukaryotes, tissue-specific and cell type-specific promoters are widely available. These promoters are so named for their ability to direct expression of a nucleic acid molecule in a given tissue or cell type within the body. Skilled artisans are well aware of numerous promoters and other regulatory elements which can be used to direct expression of nucleic acids.

In addition to sequences that facilitate transcription of the inserted nucleic acid molecule, vectors can contain origins of replication, and other genes that encode a selectable marker. For example, the neomycin-resistance (neo^{t}) gene imparts G418 resistance to cells in which it is expressed, and thus permits phenotypic selection of the transfected cells. Those of skill in the art can readily determine whether a given regulatory element or selectable marker is suitable for use in a particular experimental context.

Viral vectors that are suitable for use include, for example, retroviral, adenoviral, and adeno-associated vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

Prokaryotic or eukaryotic cells that contain and express a nucleic acid molecule that encodes a polypeptide mutant are also suitable for use. A cell is a transfected cell, i.e., a cell into which a nucleic acid molecule, for example a nucleic acid molecule encoding a mutant polypeptide, has been introduced by means of recombinant DNA techniques. The progeny of such a cell are also considered suitable for use in the methods disclosed herein.

The precise components of the expression system are not critical. For example, a polypeptide mutant can be produced in a prokaryotic host, such as the bacterium E. coli, or in a eukaryotic host, such as an insect cell (e.g., an Sf21 cell), or mammalian cells (e.g., COS cells, NIH 3T3 cells, or HeLa cells). These cells are available from many sources, including the American Type Culture Collection (Manassas, Va.). In selecting an expression system, it matters only that the components are compatible with one another. Artisans or ordinary skill are able to make such a determination. Furthermore, if guidance is required in selecting an expression system, skilled artisans may consult Ausubel et al. (Current Protocols in Molecular Biology, John Wiley and Sons, New York, N.Y., 1993) and Pouwels et al. (Cloning Vectors: A Laboratory Manual, 1985 Suppl. 1987).

The expressed polypeptides can be purified from the expression system using routine biochemical procedures, and can be used, e.g., conjugated to a lipid, as described herein.

Pharmaceutical Composition and Modes of Administration

In some embodiments, an amphiphilic ligand conjugate and CAR expressing cells (e.g., CAR T cells) are administered together (simultaneously or sequentially). In some embodiments, an amphiphilic ligand conjugate and an adjuvant (e.g., amphiphilic oligonucleotide conjugate) are administered together (simultaneously or sequentially). In some embodiments, an amphiphilic ligand conjugate, an adjuvant (e.g., amphiphilic oligonucleotide conjugate), and CAR expressing cells (e.g., CAR T cells) are administered together (simultaneously or sequentially). In some embodiments, an amphiphilic ligand conjugate and CAR expressing cells (e.g., CAR T cells) are administered separately. In some embodiments, an amphiphilic ligand conjugate and an adjuvant (e.g., amphiphilic oligonucleotide conjugate) are administered separately. In some embodiments, an amphiphilic ligand conjugate, an adjuvant (e.g., amphiphilic oligonucleotide conjugate) and CAR expressing cells (e.g., CAR T cells) are administered separately.

In some embodiments, the disclosure provides for a pharmaceutical composition comprising an amphiphilic ligand conjugate with a pharmaceutically acceptable diluent, carrier, solubilizer, emulsifier, preservative and/or adjuvant. In some embodiments, the adjuvant is an amphiphilic oligonucleotide conjugate. In some embodiments, the adjuvant is a STING agonist (e.g., CDG) In some embodiments, the adjuvant is formulated in a separate pharmaceutical composition.

In some embodiments, acceptable formulation materials preferably are nontoxic to recipients at the dosages and concentrations employed. In certain embodiments, the formulation material(s) are for s.c. and/or I.V. administration. In some embodiments, the pharmaceutical composition contains formulation materials for modifying, maintaining or preserving, for example, the pH, osmolality, viscosity, clarity, color, isotonicity, odor, sterility, stability, rate of dissolution or release, adsorption or penetration of the composition. In some embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company (1995). In certain embodiments, the formulation comprises PBS; 20 mM NaOAC, pH 5.2, 50 mM NaCl; and/or 10 mM NAOAC, pH 5.2, 9% Sucrose. In some embodiments, the optimal pharmaceutical composition is determined by one skilled in the art depending upon, for example, the intended route of administration, delivery format and desired dosage. See, for example, Remington's Pharmaceutical Sciences, supra. In some embodiments, such compositions may influence the physical state, stability, rate of in vivo release and rate of in vivo clearance of the amphiphilic conjugate.

In some embodiments, the primary vehicle or carrier in a pharmaceutical composition can be either aqueous or non-aqueous in nature. For example, in some embodiments, a suitable vehicle or carrier is water for injection, physiological saline solution or artificial cerebrospinal fluid, possibly supplemented with other materials common in compositions for parenteral administration. In some embodiments, the saline comprises isotonic phosphate-buffered saline. In certain embodiments, neutral buffered saline or saline mixed with serum albumin are further exemplary vehicles. In some embodiments, pharmaceutical compositions comprise Tris buffer of about pH 7.0-8.5, or acetate buffer of about pH 4.0-5.5, which can further include sorbitol or a suitable substitute therefore. In some embodiments, a composition comprising an amphiphilic conjugate can be prepared for storage by mixing the selected composition having the desired degree of purity with optional formulation agents (Remington's Pharmaceutical Sciences, supra) in the form of a lyophilized cake or an aqueous solution. Further, in some embodiments, a composition comprising an amphiphilic conjugate, can be formulated as a lyophilizate using appropriate excipients such as sucrose.

In some embodiments, the pharmaceutical composition can be selected for parenteral delivery. In some embodiments, the compositions can be selected for inhalation or for delivery through the digestive tract, such as orally. The preparation of such pharmaceutically acceptable compositions is within the ability of one skilled in the art.

In some embodiments, the formulation components are present in concentrations that are acceptable to the site of administration. In some embodiments, buffers are used to maintain the composition at physiological pH or at a slightly lower pH, typically within a pH range of from about 5 to about 8.

In some embodiments, when parenteral administration is contemplated, a therapeutic composition can be in the form of a pyrogen-free, parenterally acceptable aqueous solution comprising an amphiphilic conjugate, in a pharmaceutically acceptable vehicle. In some embodiments, a vehicle for parenteral injection is sterile distilled water in which an amphiphilic conjugate is formulated as a sterile, isotonic solution, properly preserved. In some embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In some embodiments, hyaluronic acid can also be used, and can have the effect of promoting sustained duration in the circulation. In some embodiments, implantable drug delivery devices can be used to introduce the desired molecule.

In some embodiments, a pharmaceutical composition can be formulated for inhalation. In some embodiments, an amphiphilic conjugate can be formulated as a dry powder for inhalation. In some embodiments, an inhalation solution comprising an amphiphilic conjugate can be formulated with a propellant for aerosol delivery. In some embodiments, solutions can be nebulized. Pulmonary administration is further described in PCT application No. PCT/US94/001875, which describes pulmonary delivery of chemically modified proteins.

In some embodiments, it is contemplated that formulations can be administered orally. In some embodiments, an amphiphilic conjugate that is administered in this fashion can be formulated with or without those carriers customarily used in the compounding of solid dosage forms such as tablets and capsules. In some embodiments, a capsule can be designed to release the active portion of the formulation at the point in the gastrointestinal tract when bioavailability is maximized and pre-systemic degradation is minimized. In some embodiments, at least one additional agent can be included to facilitate absorption of the amphiphilic conjugate. In certain embodiments, diluents, flavorings, low melting point waxes, vegetable oils, lubricants, suspending agents, tablet disintegrating agents, and binders can also be employed.

In some embodiments, a pharmaceutical composition can involve an effective quantity of an amphiphilic conjugate in a mixture with non-toxic excipients which are suitable for the manufacture of tablets. In some embodiments, by dissolving the tablets in sterile water, or another appropriate vehicle, solutions can be prepared in unit-dose form. In some embodiments, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving an amphiphilic conjugate in sustained- or controlled-delivery formulations. In some embodiments, techniques for formulating a variety of other sustained- or controlled-delivery means, such as liposome carriers, bio-erodible microparticles or porous beads and depot injections, are also known to those skilled in the art. See for example, PCT Application No. PCT/US93/00829 which describes the controlled release of porous polymeric microparticles for the delivery of pharmaceutical compositions. In some embodiments, sustained-release preparations can include semipermeable polymer matrices in the form of shaped articles, e.g. films, or microcapsules. Sustained release matrices can include polyesters, hydrogels, polylactides (U.S. Pat. No. 3,773,919 and EP 058,481), copolymers of L-glutamic acid and gamma ethyl-L-glutamate (Sidman et al., Biopolymers, 22:547-556 (1983)), poly (2-hydroxyethyl-methacrylate) (Langer et al., J. Biomed. Mater. Res., 15: 167-277 (1981) and Langer, Chem. Tech., 12:98-105 (1982)), ethylene vinyl acetate (Langer et al., supra) or poly-D(−)-3-hydroxybutyric acid (EP 133,988). In some embodiments, sustained release compositions can also include liposomes, which can be prepared by any of several methods known in the art. See, e.g., Eppstein et al, Proc. Natl. Acad. Sci. USA, 82:3688-3692 (1985); EP 036,676; EP 088,046 and EP 143,949.

In some embodiments, the pharmaceutical composition to be used for in vivo administration is sterile. In some embodiments, sterility is accomplished by filtration through sterile filtration membranes. In certain embodiments, where the composition is lyophilized, sterilization using this method is conducted either prior to or following lyophilization and reconstitution. In some embodiments, the composition for parenteral administration is stored in lyophilized form or in a solution. In some embodiments, parenteral compositions are placed into a container having a sterile access port, for example, an intravenous solution bag or vial having a stopper pierceable by a hypodermic injection needle.

In some embodiments, once the pharmaceutical composition has been formulated, it is stored in sterile vials as a solution, suspension, gel, emulsion, solid, or as a dehydrated or lyophilized powder. In some embodiments, such formulations are stored either in a ready-to-use form or in a form (e.g., lyophilized) that is reconstituted prior to administration.

In some embodiments, kits are provided for producing a single-dose administration unit. In some embodiments, the kit can contain both a first container having a dried protein and a second container having an aqueous formulation. In someembodiments, kits containing single and multi-chambered pre-filled syringes (e.g., liquid syringes and lyosyringes) are included.

In some embodiments, the effective amount of a pharmaceutical composition comprising an amphiphilic conjugate to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which an amphiphilic conjugate is being used, the route of administration, and the size (body weight, body surface or organ size) and/or condition (the age and general health) of the patient. In some embodiments, the clinician can titer the dosage and modify the route of administration to obtain the optimal therapeutic effect.

In some embodiments, the frequency of dosing will take into account the pharmacokinetic parameters of the amphiphilic conjugate, in the formulation used. In some embodiments, a clinician will administer the composition until a dosage is reached that achieves the desired effect. In some embodiments, the composition can therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. In some embodiments, appropriate dosages can be ascertained through use of appropriate dose-response data.

In some embodiments, the route of administration of the pharmaceutical composition is in accord with known methods, e.g. orally, through injection by intravenous, intraperitoneal, intracerebral (intra-parenchymal), intracerebroventricular, intramuscular, subcutaneously, intraocular, intraarterial, intraportal, or intralesional routes; by sustained release systems or by implantation devices. In certain embodiments, the compositions can be administered by bolus injection or continuously by infusion, or by implantation device. In certain embodiments, individual elements of the combination therapy may be administered by different routes.

In some embodiments, the composition can be administered locally via implantation of a membrane, sponge or another appropriate material onto which the desired molecule has been absorbed or encapsulated. In some embodiments, where an implantation device is used, the device can be implanted into any suitable tissue or organ, and delivery of the desired molecule can be via diffusion, timed-release bolus, or continuous administration. In some embodiments, it can be desirable to use a pharmaceutical composition comprising an amphiphilic conjugate in an ex vivo manner. In such instances, cells, tissues and/or organs that have been removed from the patient are exposed to a pharmaceutical composition comprising an amphiphilic conjugate, after which the cells, tissues and/or organs are subsequently implanted back into the patient.

In some embodiments, an amphiphilic conjugate can be delivered by implanting certain cells that have been genetically engineered, using methods such as those described herein, to express and secrete the conjugate. In some embodiments, such cells can be animal or human cells, and can be autologous, heterologous, or xenogeneic. In some embodiments, the cells can be immortalized. In some embodiments, in order to decrease the chance of an immunological response, the cells can be encapsulated to avoid infiltration of surrounding tissues. In some embodiments, the encapsulation materials are typically biocompatible, semi-permeable polymeric enclosures or membranes that allow the release of the protein product(s) but prevent the destruction of the cells by the patient's immune system or by other detrimental factors from the surrounding tissues.

Methods of Use

In some embodiments, the disclosure provides methods of expanding or activating CAR effector cells (e.g., CAR-T cells) in vivo in a subject, comprising administering a composition comprising an amphiphilic lipid conjugate described herein.

In some embodiments, the disclosure provides methods of stimulation proliferation of CAR effector cells (e.g., CAR-T cells) in vivo in a subject, comprising administering a composition comprising an amphiphilic lipid conjugate described herein.

Methods for determining expansion, activation and proliferation of cells are known to those of skill in the art. For example, the number of cells at a specified location (e.g., lymph nodes, blood, tumor) can be determined by isolating the cells and analyzing them via flow cytometry. In some embodiments, the cells are stained with appropriate markers, such as activation markers (e.g., CD80, CD86, 41BBL, ICOSL or OX40L) and/or proliferation markers (e.g., Ki67). In some embodiments, the number of cells is measured by introducing a dye (e.g., crystal violet) into cells, and measuring the dilution of the dye over time, wherein dilution indicates cell proliferation.

In some embodiments, the disclosure provides methods for treating a subject having a disease, disorder or condition associated with expression or elevated expression of an antigen, comprising administering to the subject CAR effector cells (e.g., CAR-T cells) targeted to the antigen, and an amphiphilic lipid conjugate.

In some embodiments, the subject is administered the CAR effector cells (e.g., CAR-T cells) prior to receiving the amphiphilic lipid conjugate. In some embodiments, the subject is administered the CAR effector cells (e.g., CAR-T cells) after receiving the amphiphilic lipid conjugate. In some embodiments, the subject is administered the CAR effector cells (e.g., CAR-T cells) and the amphiphilic lipid conjugate sequentially or simultaneously.

In some embodiments, wherein the CAR comprises a tag binding domain, the methods disclosed herein further comprise administering a formulation of tagged proteins, wherein the tag binding domain binds the tagged proteins. In some embodiments, the protein of the tagged protein is an antibody or an antigen-binding fragment. In some embodiments, the tag binding domain is an antibody or antigen-binding fragment thereof. In some embodiments, the formulation of tagged proteins is administered to the subject prior to administration of the CAR effector cell (e.g., CAR T cells) and amphiphilic ligand conjugate. In some embodiments, the formulation of tagged proteins is administered to the subject concurrently (simultaneously or sequentially) with the CAR effector cells (e.g., CAR T cells) and amphiphilic ligand conjugate. In some embodiments, the formulation of tagged proteins is administered to the subject after administration of the CAR effector cells (e.g., CAR T cells) and amphiphilic ligand conjugate.

Cancer and Cancer Immunotherapy

In some embodiments, the amphiphilic ligand conjugate described herein is useful for treating a disorder associated with abnormal apoptosis or a differentiative process (e.g., cellular proliferative disorders (e.g., hyperproliferaetive disorders) or cellular differentiative disorders, such as cancer). Non-limiting examples of cancers that are amenable to treatment with the methods of the present invention are described below.

Examples of cellular proliferative and/or differentiative disorders include cancer (e.g., carcinoma, sarcoma, metastatic disorders or hematopoietic neoplastic disorders, e.g., leukemias). A metastatic tumor can arise from a multitude of primary tumor types, including but not limited to those of prostate, colon, lung, breast and liver. Accordingly, the compositions used herein, comprising, an amphiphilic ligand conjugate can be administered to a patient who has cancer.

As used herein, we may use the terms “cancer” (or “cancerous”), “hyperproliferative,” and “neoplastic” to refer to cells having the capacity for autonomous growth (i.e., an abnormal state or condition characterized by rapidly proliferating cell growth). Hyperproliferative and neoplastic disease states may be categorized as pathologic (i.e., characterizing or constituting a disease state), or they may be categorized as non-pathologic (i.e., as a deviation from normal but not associated with a disease state). The terms are meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasm” are used to refer to malignancies of the various organ systems, including those affecting the lung, breast, thyroid, lymph glands and lymphoid tissue, gastrointestinal organs, and the genitourinary tract, as well as to adenocarcinomas which are generally considered to include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. The amphiphilic ligand conjugate can be used to treat patients who have, who are suspected of having, or who may be at high risk for developing any type of cancer, including renal carcinoma or melanoma, or any viral disease. Exemplary carcinomas include those forming from tissue of the cervix, lung, prostate, breast, head and neck, colon and ovary. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias (e.g., erythroblastic leukemia and acute megakaryoblastic leukemia). Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit. Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macro globulinemia (WM). Additional forms of malignant lymphomas include, but are not limited to non-Hodgkin lymphoma and variants thereof, peripheral T cell lymphomas, adult T cell leukemia/lymphoma (ATL), cutaneous T cell lymphoma (CTCL), large granular lymphocytic leukemia (LGF), Hodgkin's disease and Reed-Sternberg disease.

It will be appreciated by those skilled in the art that amounts for an amphiphilic conjugate that is sufficient to reduce tumor growth and size, or a therapeutically effective amount, will vary not only on the particular compound or composition selected, but also with the route of administration, the nature of the condition being treated, and the age and condition of the patient, and will ultimately be at the discretion of the patient's physician or pharmacist. The length of time during which the compound used in the instant method will be given varies on an individual basis.

In some embodiments, the disclosure provides methods of reducing or decreasing the size of a tumor, or inhibiting a tumor growth in a subject in need thereof, comprising administering to the subject an amphiphilic lipid conjugate described herein, wherein the subject is receiving or has received CAR effector cell therapy (e.g., CAR-T cell therapy). In some embodiments, the disclosure provides methods for inducing an anti-tumor response in a subject with cancer, comprising administering to the subject an amphiphilic lipid conjugate described herein, wherein the subject is receiving or has received CAR effector cell therapy (e.g., CAR-T cell therapy).

In some embodiments, the disclosure provides methods for stimulating an immune response to a target cell population or target tissue expressing an antigen in a subject, comprising administering effector CAR cells (e.g., CAR-T cells) targeted to the antigen, and an amphiphilic lipid conjugate. In some embodiments, the immune response is a T-cell mediated immune response. In some embodiments, the immune response is an anti-tumor immune response. In some embodiments, the target cell population or target tissue is tumor cells or tumor tissue.

It will be appreciated by those skilled in the art that reference herein to treatment extends to prophylaxis as well as the treatment of the noted cancers and symptoms.

Infectious Diseases

In some embodiments, an amphiphilic lipid conjugate disclosed herein is useful for treating acute or chronic infectious diseases. Because viral infections are cleared primarily by T-cells, an increase in T-cell activity is therapeutically useful in situations where more rapid or thorough clearance of an infective viral agent would be beneficial to an animal or human subject.

Recently, CAR-T cell therapy has been investigated for its usefulness in treating viral infections, such as human immunodeficiency virus (HIV), as described in PCT Publication No. WO 2015/077789; Hale et al., (2017) Engineering HIV-Resistant, Anti-HIV Chimeric Antigen Receptor T Cells. Molecular Therapy, Vol. 25(3): 570-579; Liu et al., (2016). ABSTRACT, Journal of Virology, 90(21), 9712-9724; Liu et al., (2015). ABSTRACT. Journal of Virology, 89(13), 6685-6694; Sahu et al., (2013). Virology, 446(1-2), 268-275.

Thus, in some embodiments the amphiphilic ligand conjugates are administered for the treatment of local or systemic viral infections, including, but not limited to, immunodeficiency (e.g., HIV), papilloma (e.g., V), herpes (e.g., HSV), encephalitis, influenza (e.g., human influenza virus A). and common cold (e.g., human rhinovirus) viral infections. In some embodiments, pharmaceutical formulations including the amphiphilic ligand conjugates are administered topically to treat viral skin diseases such as herpes lesions or shingles, or genital warts. In some embodiments, the amphiphilic ligand conjugates are administered to treat systemic viral diseases, including, but not limited to, AIDS, influenza, the common cold, or encephalitis.

In some embodiments, the disclosure provides methods for increasing proliferation of CAR effector cells (e.g., CAR-T cells) in vivo, in a subject with a viral infection, comprising administering a composition comprising an amphiphilic ligand conjugate, wherein the CAR comprises a viral peptide binding domain (e.g., a HIV Env binding domain), and wherein the amphiphilic ligand conjugate comprises the viral peptide (e.g., HIV Env).

In some embodiments, the disclosure provides methods for expanding CAR effector cells (e.g., CAR-T cells) in viva, in a subject with a viral infection, comprising administering a composition comprising an amphiphilic ligand conjugate, wherein the CAR comprises a viral peptide binding domain (e.g., a HIV Env binding domain), and wherein the amphiphilic ligand conjugate comprises the viral peptide (e.g., HIV Env).

In some embodiments, the disclosure provides methods of reducing a viral infection in a subject in need thereof, comprising administering to the subject an amphiphilic lipid conjugate described herein, wherein the subject is receiving or has received CAR effector cell therapy (e.g., CAR-T cell therapy). In some embodiments, the disclosure provides methods for inducing an anti-viral response in a subject with cancer, comprising administering to the subject an amphiphilic lipid conjugate described herein, wherein the subject is receiving or has received CAR effector cell therapy (e.g., CAR-T cell therapy).

It will be appreciated by those skilled in the art that reference herein to treatment extends to prophylaxis as well as the treatment of the noted infections and symptoms.

Kits

Provided herein are kits comprising at least an amphiphilic ligand conjugate described herein and instructions for use. In some embodiments, the kits comprise, in a suitable container, an amphiphilic ligand conjugate, one or more controls, and various buffers, reagents, enzymes and other standard ingredients well known in the art. In some embodiments, the kits further comprise an adjuvant (e.g., an amphiphilic oligonucleotide conjugate or a STING agonist (e.g., CDG)). Accordingly, in some embodiments, the amphiphilic ligand conjugate and adjuvant are in the same vial. In some embodiments, the amphiphilic ligand conjugate and adjuvant are in separate vials.

In some embodiments, the container is at least one vial, well, test tube, flask, bottle, syringe, or other container means, into which an amphiphilic ligand conjugate may be placed, and in some instances, suitably aliquoted. When an additional component is provided, the kit can contain additional containers into which this compound may be placed. The kits can also include a means for containing an amphiphilic ligand conjugate, and any other reagent containers in close confinement for commercial sale. Such containers may include injection or blow-molded plastic containers into which the desired vials are retained. Containers and/or kits can include labeling with instructions for use and/or warnings.

In some embodiments, the disclosure provides a kit comprising a container comprising a composition comprising an amphiphilic ligand conjugate described herein, an optional pharmaceutically acceptable carrier, and a package insert comprising instructions for administration of the composition for treating or delaying progression of cancer in an individual receiving CAR-T cell therapy In some embodiments, the kit further comprises an adjuvant and instructions for administration of the adjuvant for treating or delaying progression of cancer in an individual receiving CAR-T cell therapy. In some embodiments, the adjuvant is an amphiphilic oligonucleotide conjugate described herein. In some embodiments, the adjuvant is a STING agonist. In some embodiments, the adjuvant is CDG.

In some embodiments, the disclosure provides a kit comprising a medicament comprising a composition comprising an amphiphilic ligand conjugate described herein, an optional pharmaceutically acceptable carrier, and a package insert comprising instructions for administration of the medicament alone or in combination with a composition comprising an adjuvant and an optional pharmaceutically acceptable carrier, for treating or delaying progression of cancer in an individual receiving CAR-T cell therapy.

In some embodiments, the disclosure provides a kit comprising a container comprising a composition comprising an amphiphilic ligand conjugate described herein, an optional pharmaceutically acceptable carrier, and a package insert comprising instructions for administration of composition vaccine for expanding CAR-T cells in an individual receiving CAR-T cell therapy. In some embodiments, the kit further comprises an adjuvant and instructions for administration of the adjuvant for expanding CAR-T cells in an individual receiving CAR-T cell therapy. In some embodiments, the adjuvant is an amphiphilic oligonucleotide conjugate described herein. In some embodiments, the adjuvant is a STING agonist. In some embodiments, the adjuvant is CDG.

In some embodiments, the disclosure provides a kit comprising a medicament comprising a composition comprising an amphiphilic ligand conjugate described herein, an optional pharmaceutically acceptable carrier, and a package insert comprising instructions for administration of the medicament alone or in combination with a composition comprising an adjuvant and an optional pharmaceutically acceptable carrier, for expanding CAR-T cells in an individual receiving CAR-T cell therapy. In some embodiments, the adjuvant is an amphiphilic oligonucleotide conjugate described herein. In some embodiments, the adjuvant is a STING agonist. In some embodiments, the adjuvant is CDG.

In some embodiments, the disclosure provides a kit comprising a container comprising a composition comprising an amphiphilic ligand conjugate described herein, an optional pharmaceutically acceptable carrier, and a package insert comprising instructions for administration of the composition for increasing proliferation of CAR-T cells in an individual receiving CAR T cell therapy. In some aspects, the kit further comprises an adjuvant and instructions for administration of the adjuvant for increasing proliferation of CAR-T cells in an individual receiving CAR-T cell therapy. In some embodiments, the adjuvant is an amphiphilic oligonucleotide conjugate described herein. In some embodiments, the adjuvant is a STING agonist. In some embodiments, the adjuvant is CDG.

In some embodiments, the disclosure provides a kit comprising a medicament comprising a composition comprising an amphiphilic ligand conjugate described herein, an optional pharmaceutically acceptable carrier, and a package insert comprising instructions for administration of the medicament alone or in combination with a composition comprising an adjuvant and an optional pharmaceutically acceptable carrier, for increasing proliferation of CAR-T cells in an individual receiving CAR-T cell therapy. In some embodiments, the adjuvant is an amphiphilic oligonucleotide conjugate described herein. In some embodiments, the adjuvant is a STING agonist. In some embodiments, the adjuvant is CDG.

In some embodiments, any of the kits described herein further comprise CAR-T cells comprising a CAR that binds to the CAR ligand present in the amphiphilic ligand conjugate.

### OTHER EMBODIMENTS OF THE DISCLOSURE

Throughout this section, the term embodiment is abbreviated as ‘E’ followed by an ordinal. For example, E1 is equivalent to Embodiment 1.

E1. A method of expanding chimeric antigen receptor (CAR) T cells or increasing proliferation of CAR T cells in vivo in a subject, comprising administering a composition in an amount sufficient to expand CAR T cells in the subject, wherein the composition comprises an amphiphilic ligand conjugate comprising a lipid, a CAR ligand, and optionally a linker.
E2. The method of embodiment 1, wherein the amphiphilic ligand conjugate binds albumin under physiological conditions.
E3. The method of embodiment 2, wherein proliferation of CAR(−) T cells is not increased in the subject.
E4. A method of reducing or decreasing a size of a tumor or inhibiting a tumor growth in a subject in need thereof, comprising administering to the subject a composition, wherein the subject is receiving or has received chimeric antigen receptor (CAR) T cell therapy, and wherein the composition comprises an amphiphilic ligand conjugate comprising a lipid, a CAR ligand, and optionally a linker.
E5. A method of inducing an anti-tumor response in a subject with cancer, comprising administering to the subject a composition, wherein the subject is receiving or has received chimeric antigen receptor (CAR) T cell therapy, and wherein the composition comprises an amphiphilic ligand conjugate comprising a lipid, a CAR ligand, and optionally a linker.
E6. A method of stimulating an immune response to a target cell population or target tissue expressing an antigen in a subject, the method comprising administering to the subject chimeric antigen receptor (CAR) T cells targeted to the antigen and a composition, wherein the composition comprises an amphiphilic ligand conjugate comprising a lipid, a CAR ligand, and optionally a linker.
E7. The method of embodiment 6, wherein the immune response is a T-cell mediated immune response or an anti-tumor immune response.
E8. The method of embodiment 6 or 7, wherein the target cell population or target tissue is tumor cells or tumor tissue.
E9. A method of treating a subject having a disease, disorder or condition associated with expression or elevated expression of an antigen, comprising administering to the subject chimeric antigen receptor (CAR) T cells targeted to the antigen, and composition, wherein the composition comprises an amphiphilic ligand conjugate comprising a lipid, a CAR ligand, and optionally a linker.
E10. The method of any one of embodiments 1-3, wherein the subject is administered the composition prior to receiving CAR T cells.
E11. The method of any one of embodiments 1-3, wherein the subject is administered the composition after receiving CAR T cells.
E12. The method of any one of embodiments 1-3, wherein the composition and CAR T cells are administered simultaneously.
E13. The method of any one of the preceding embodiments, wherein CAR T cells comprise one co-stimulation domain.
E14. The method of embodiment 13, wherein the one co-stimulation domain is CD28 or 4-1BB.
E15. The method of any one of embodiments 1-14, wherein the amphiphilic ligand conjugate is trafficked to the lymph nodes.
E16. The method of any one of embodiments 1-14, wherein the amphiphilic ligand conjugate is trafficked to the inguinal lymph node and auxiliary lymph node.
E17. The method of any one of embodiments 1-16, wherein the amphiphilic ligand conjugate is inserted into the membrane of antigen presenting cells upon trafficking to the lymph nodes.
E18. The method of embodiment 17, wherein the antigen presenting cells are medullary macrophages, CD8+ dendritic cells, and/or CDllb+ dendritic cells.
E19. The method of any one of embodiments 1-18, wherein the CAR ligand is retained in the lymph nodes for at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, or at least 25 days.
E20. The method of any one of embodiments 1-19, wherein the composition further comprises an adjuvant.
E21. The method of embodiment 20, wherein the adjuvant is an amphiphilic oligonucleotide conjugate comprising an immunostimulatory oligonucleotide conjugated to a lipid, with or without a linker, and optionally a polar compound.
E22. The method of embodiment 21, wherein the immunostimulatory oligonucleotide binds a pattern recognition receptor.
E23. The method of embodiment 22, wherein the immunostimulatory oligonucleotide comprises CpG.
E24. The method of embodiment 21, wherein the immunostimulatory oligonucleotide is a ligand for a toll-like receptor.
E25. The method of any one of embodiments 1-20, wherein the linker is selected from the group consisting of hydrophilic polymers, a string of hydrophilic amino acids, polysaccharides, or a combination thereof.
E26. The method of any one of embodiments 1-20, wherein the linker comprises “N” consecutive polyethylene glycol units, wherein N is between 25-50.
E27. The method of any one of embodiments 1-26, wherein the lipid is a diacyl lipid.
E28. The method of any one of embodiments 21-24, wherein the linker is an oligonucleotide linker.
E29. The method of embodiment 28, wherein the oligonucleotide linker comprises “N” consecutive guanines, wherein N is between 0-2.
E30. The method of any one of embodiments 21-24 and 28-29, wherein the lipid is diacyl lipid.
E31. The method of any one of embodiments 1-30, wherein the CAR ligand is a tumor associated antigen, and wherein the CAR comprises a tumor associated antigen binding domain.
E32. The method of any one of embodiments 1-30, wherein the CAR ligand is a tag, and wherein the CAR comprises a tag binding domain.
E33. The method of embodiment 32, wherein the tag is selected from the group consisting of fluorescein isothiocyanate (FITC), streptavidin, biotin, dinitrophenol, peridinin chlorophyll protein complex, green fluorescent protein, phycoerythrin (PE), horse radish peroxidase, palmitoylation, nitrosylation, alkalanine phosphatase, glucose oxidase, and maltose binding protein.
E34. The method of embodiment 32 or 33, further comprising administering a formulation of tagged proteins, and wherein the tag binding domain binds the tagged proteins.
E35. The method of embodiment 34, wherein the protein of the tagged protein is an antibody or an antigen-binding fragment thereof.
E36. The method of embodiment 34 or 35, wherein the tag binding domain is an antibody or an antigen-binding fragment thereof.
E37. The method of any one of embodiments 34-36, wherein the formulation of tagged proteins is administered to the subject prior to administration of the CAR T cells and composition comprising the amphiphilic ligand conjugate.
E38. The method of any one of embodiments 34-36, wherein the formulation of tagged proteins is administered to the subject concurrently with administration of the CAR T cells and composition comprising the amphiphilic ligand conjugate.
E39. The method of any one of embodiments 34-36, wherein the formulation of tagged proteins is administered to the subject after administration of the CAR T cells and composition comprising the amphiphilic ligand conjugate.
E40. The method of any one of embodiments 37-39, wherein the CAR T cells are administered prior to administration of the composition comprising the amphiphilic ligand conjugate.
E41. The method of any one of embodiments 37-39, wherein the CAR T cells are administered after administration of the composition comprising the amphiphilic ligand conjugate.
E42. The method of any one of embodiments 37-39, wherein the CAR T cells are administered concurrently with administration of the composition comprising the amphiphilic ligand conjugate.
E43. The method of any one of embodiments 1-3 and 6-42, wherein the subject has cancer.
E44. The method of any one of embodiments 1-43, wherein the subject is a human.
E45. A composition comprising an amphiphilic ligand conjugate, wherein the amphiphilic ligand conjugate comprises a chimeric antigen receptor (CAR) ligand, a lipid, and optionally a linker, and a pharmaceutically acceptable carrier.
E46. The composition of embodiment 45, wherein the linker is selected from the group consisting of hydrophilic polymers, a string of hydrophilic amino acids, polysaccharides, or a combination thereof.
E47. The composition of embodiment 45, wherein the linker comprises “N” consecutive polyethylene glycol units, wherein N is between 25-50.
E48. The composition of any one of embodiments 45-47, wherein the lipid is diacyl lipid.
E49. The composition of any one of embodiments 45-48, wherein the CAR ligand is a tag.
E50. The composition of embodiment 49, wherein the tag is selected from the group consisting of fluorescein isothiocyanate (FITC), streptavidin, biotin, dinitrophenol, peridinin chlorophyll protein complex, green fluorescent protein, phycoerythrin (PE), horse radish peroxidase, palmitoylation, nitrosylation, alkalanine phosphatase, glucose oxidase, and maltose binding protein.
E51. An immunogenic composition, comprising the composition of any one of embodiments 45-50, and an adjuvant.
E52. The immunogenic composition of embodiment 51, wherein the adjuvant is an amphiphilic oligonucleotide conjugate comprising an immunostimulatory oligonucleotide conjugated to a lipid with or without a linker, and optionally a polar compound.
E53. The immunogenic composition of embodiment 52, wherein the immunostimulatory oligonucleotide binds a pattern recognition receptor.
E54. The immunogenic composition of embodiment 53, wherein the immunostimulatory oligonucleotide comprises CpG.
E55. The immunogenic composition of embodiment 52, wherein the immunostimulatory oligonucleotide is a ligand for a toll-like receptor.
E56. The immunogenic composition of any one of embodiments 52-55, wherein the lipid is a diacyl lipid.
E57. The immunogenic composition of any one of embodiments 52-56, wherein the linker is an oligonucleotide linker.
E58. The immunogenic composition of embodiment 57, wherein the oligonucleotide linker comprises “N” consecutive guanines, wherein N is between 0-2.
E59. A kit comprising a container comprising a composition comprising an amphiphilic ligand conjugate, an optional pharmaceutically acceptable carrier, and a package insert comprising instructions for administration of the composition for treating or delaying progression of cancer in an individual receiving CAR T cell therapy, wherein the amphiphilic ligand conjugate comprises a lipid, a CAR ligand, and optionally a linker.
E60. The kit of embodiment 59, further comprising an adjuvant and instructions for administration of the adjuvant for treating or delaying progression of cancer in an individual receiving chimeric antigen receptor (CAR) T cell therapy.
E61. The kit of embodiment 60, wherein the adjuvant is an amphiphilic oligonucleotide conjugate comprising an immunostimulatory oligonucleotide conjugated to a lipid with or without a linker, and optionally a polar compound.
E62. A kit comprising a medicament comprising a composition comprising an amphiphilic ligand conjugate, an optional pharmaceutically acceptable carrier, and a package insert comprising instructions for administration of the medicament alone or in combination with a composition comprising an adjuvant and an optional pharmaceutically acceptable carrier, for treating or delaying progression of cancer in an individual receiving chimeric antigen receptor (CAR) T cell therapy, wherein the amphiphilic ligand conjugate comprises a lipid, a CAR ligand, and optionally a linker.
E63. A kit comprising a container comprising a composition comprising an amphiphilic ligand conjugate, an optional pharmaceutically acceptable carrier, and a package insert comprising instructions for administration of composition vaccine for expanding CAR T cells in an individual receiving CAR T cell therapy, wherein the amphiphilic ligand conjugate comprises a lipid, a CAR ligand, and optionally a linker.
E64. The kit of embodiment 63, further comprising an adjuvant and instructions for administration of the adjuvant for expanding CAR T cells in an individual receiving chimeric antigen receptor (CAR) T cell therapy.
E65. The kit of embodiment 64, wherein the adjuvant is an amphiphilic oligonucleotide conjugate comprising an immunostimulatory oligonucleotide conjugated to a lipid with or without a linker, and optionally a polar compound.
E66. A kit comprising a medicament comprising a composition comprising an amphiphilic ligand conjugate, an optional pharmaceutically acceptable carrier, and a package insert comprising instructions for administration of the medicament alone or in combination with a composition comprising an adjuvant and an optional pharmaceutically acceptable carrier, for expanding CAR T cells in an individual receiving CAR T cell therapy, wherein the amphiphilic ligand conjugate comprises a lipid, a CAR ligand, and optionally a linker.
E67. A kit comprising a container comprising a composition comprising an amphiphilic ligand conjugate, an optional pharmaceutically acceptable carrier, and a package insert comprising instructions for administration of the composition for increasing proliferation of CAR T cells in an individual receiving CAR T cell therapy, wherein the amphiphilic ligand conjugate comprises a lipid, a CAR ligand, and optionally a linker.
E68. The kit of embodiment 67, further comprising an adjuvant and instructions for administration of the adjuvant for increasing proliferation of CAR T cells in an individual receiving chimeric antigen receptor (CAR) T cell therapy.
E69. The kit of embodiment 66 or 68, wherein the adjuvant is an amphiphilic oligonucleotide conjugate comprising an immunostimulatory oligonucleotide conjugated to a lipid with or without a linker, and optionally a polar compound.
E70. A kit comprising a medicament comprising a composition comprising an amphiphilic ligand conjugate, an optional pharmaceutically acceptable carrier, and a package insert comprising instructions for administration of the medicament alone or in combination with a composition comprising an adjuvant and an optional pharmaceutically acceptable carrier, for increasing proliferation of CAR T cells in an individual receiving CAR T cell therapy, wherein the amphiphilic ligand conjugate comprises a lipid, a CAR ligand, and optionally a linker.
E71. Use of a composition of any one of embodiments 45-50, an immunogenic composition of any one of embodiments 51-58, or a kit of any one of embodiments 59-70, for use in expanding CAR T cells in vivo in a subject.
E72. Use of a composition of any one of embodiments 45-50, an immunogenic composition of any one of embodiments 51-58, or a kit of any one of embodiments 59-70, for use in increasing proliferation of CAR T cells in vivo in a subject.
E73. Use of a composition of any one of embodiments 45-50, an immunogenic composition of any one of embodiments 51-58, or a kit of any one of embodiments 59-70, for use in treating or delaying progression of cancer in an individual.
E74. Use of a composition of any one of embodiments 45-50, in the manufacture of a medicament for treating or delaying progression of cancer in an individual, wherein the medicament comprises the composition, and an optional pharmaceutically acceptable carrier.
E75. A composition comprising an amphiphilic ligand conjugate, wherein the amphiphilic ligand conjugate comprises a lipid conjugated to fluorescein isothiocyanate (FITC) via a polyethylene glycol moiety.
E76. The composition of embodiment 75, wherein the lipid is 1,2-distearoyl-sn-glycero-3-phosphoethanolamine (DSPE) and wherein the polyethylene glycol moiety is PEG-2000.
E77. An immunogenic composition comprising an amphiphilic ligand conjugate and an adjuvant, wherein the amphiphilic ligand conjugate comprises a lipid, a CAR ligand, and optionally a linker, and wherein the adjuvant is an amphiphilic oligonucleotide conjugate comprising an immunostimulatory oligonucleotide conjugated to a lipid, with or without a linker, and optionally a polar compound.
E78. An immunogenic composition comprising an amphiphilic ligand conjugate and an adjuvant, wherein the amphiphilic ligand conjugate comprises a lipid, a CAR ligand, and optionally a linker, wherein the CAR ligand is a tag, and wherein the adjuvant is an amphiphilic oligonucleotide conjugate comprising an immunostimulatory oligonucleotide conjugated to a lipid, with or without a linker, and optionally a polar compound.
E79. The method of any one of embodiments 4-44, wherein the amphiphilic ligand conjugate binds to albumin under physiological conditions.
E80. The method of any one of embodiments 21-24 and 27-44, wherein the amphiphilic oligonucleotide conjugate binds to albumin under physiological conditions.
E81. The method of any one of embodiments 1-44, wherein the method comprises administering the composition comprising an amphiphilic ligand conjugate parenterally at a non-tumor draining lymph node, parenterally at a tumor-draining lymph node, or intratumorally.
E82. The method of embodiment 6, wherein the target cell population or target tissue is a population of cells or tissue infected with a virus.
E83. The method of embodiment 82, wherein the virus is human immunodeficiency virus (HIV).
E84. The method of embodiment 82 or 83, wherein the immune response is a T-cell mediated immune response.
E85. The method of embodiment 9, wherein the antigen is a viral antigen or caner antigen.
E86. A kit comprising a container comprising a composition comprising an amphiphilic ligand conjugate, an optional pharmaceutically acceptable carrier, and a package insert comprising instructions for administration of the composition for treating or delaying progression of a viral infection in an individual receiving CAR T cell therapy, wherein the amphiphilic ligand comprises a lipid, a CAR ligand, and optionally a linker.
E87. The kit of embodiment 86, further comprising an adjuvant and instructions for administration of the adjuvant for treating or delaying progression of a viral infection in an individual receiving CAR T cell therapy.
E88. The kit of embodiment 87, wherein the adjuvant is an amphiphilic oligonucleotide conjugate comprising and immunostimulatory oligonucleotide conjugated to a lipid with or without a linker, and optionally a polar compound.
E89. The kit of any one of embodiments 59-70 and 86-88, wherein the amphiphilic ligand conjugate comprises a linker selected from the group consisting of hydrophilic polymers, a string of hydrophilic amino acids, polysaccharides, or a combination thereof.
E90. The kit of any one of embodiments 59-70 and 86-88, wherein the amphiphilic ligand conjugate comprises a linker comprising “N” consecutive polyethylene glycol units, wherein N is between 25-50.
E91. The kit of any one of embodiments 59-70 and 86-90, wherein the lipid is a diacyl lipid.
E92. The kit of any one of embodiments 61, 65, 69 or 88, wherein the amphiphilic oligonucleotide conjugate comprises an oligonucleotide linker.
E93. The kit of embodiment 92, wherein the oligonucleotide linker comprises “N” consecutive guanines, wherein N is between 0-2.
E94. The kit of any one of embodiments 59-70 and 89-93, wherein the CAR ligand is a tumor associated antigen, and wherein the CAR comprises a tumor associated antigen binding domain.
E95. The kit of any one of embodiments 59-70 and 89-93, wherein the CAR ligand is a tag, and wherein the CAR comprises a tag binding domain.
E96. The kit of embodiment 95, wherein the tag is selected from the group consisting of fluorescein isothiocyanate (FITC), streptavidin, biotin, dinitrophenol, peridinin chlorophyll protein complex, green fluorescent protein, phycoerythrin (PE), horse radish peroxidase, palmitoylation, nitrosylation, alkalanine phosphatase, glucose oxidase, and maltose binding protein.
E97. The kit of embodiment 95 or 96, wherein the kit further comprises a formulation of tagged proteins and instructions for administration of the formulation of tagged proteins, wherein the tag binding domain binds the tagged proteins.
E98. The kit of embodiment 97, wherein the protein of the tagged protein is an antibody or an antigen-binding fragment thereof.
E99. The immunogenic composition of embodiment 77 or 78, wherein the amphiphilic ligand conjugate comprises a linker selected from the group consisting of hydrophilic polymers, a string of hydrophilic amino acids, polysaccharides, or a combination thereof.
E100. The immunogenic composition of embodiment 77 or 78, wherein the amphiphilic ligand conjugate comprises a linker comprising “N” consecutive polyethylene glycol units, wherein N is between 25-50.
E101. The immunogenic composition of embodiments 77, 78, 99 or 100, wherein the lipid is a diacyl lipid.
E102. The immunogenic composition of embodiments 77 or 99-101, wherein the CAR ligand is a tumor associated antigen or a viral antigen.
E103. The immunogenic composition of embodiments 77, 78 or 99-102, wherein the amphiphilic oligonucleotide conjugate comprises an oligonucleotide linker.
E104. The immunogenic composition of embodiment 103, wherein the oligonucleotide linker comprises “N” consecutive guanines, wherein N is between 0-2.
E105. The immunogenic composition of any one of embodiments 78, 99-101 and 103-104, wherein the tag is selected from the group consisting of fluorescein isothiocyanate (FITC), streptavidin, biotin, dinitrophenol, peridinin chlorophyll protein complex, green fluorescent protein, phycoerythrin (PE), horse radish peroxidase, palmitoylation, nitrosylation, alkalanine phosphatase, glucose oxidase, and maltose binding protein.

The present disclosure is further illustrated by the following examples, which should not be construed as further limiting. The contents of all figures and all references, patents and published patent applications cited throughout this application are expressly incorporated herein by reference.

### EXAMPLES

Below are examples of specific embodiments for carrying out the methods described herein. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### Example 1: Generation of DSPE-PEG-FITC and DSPE-PEG-Peptide/Protein Ligand

Due to the poor persistence of CAR-T cells in some patient populations and the failure of CAR-T therapy to induce optimal response in solid tumors, it was hypothesized that more potent CAR-T cell expansion and enhanced functionality can be achieved by stimulation through the CAR itself. To accomplish this, albumin-binding phospholipid-polymers were utilized, as previously described (Liu, H., Moynihan, K. D., Zheng, Y., Szeto, G. L., Li, A. V., Huang, B., Irvine, D. J. (2014). Structure-based programming of lymph-node targeting in molecular vaccines. Nature, 507(7493), 519-522.). Specifically, a small molecule, peptide or protein ligand for a CAR is attached to a polymer-lipid tail, as shown in FIG. 1A, to form an amphiphile vaccine.

Initially, retargetable CAR was employed, wherein the chimeric antigen receptor recognizes the small molecule fluorescein (FITC), which is targeted against tumors through a FITC-conjugated anti-tumor antibody (Ma, J. S., Kim, J. Y., Kazane, S. A., Choi, S. H., Yun, H. Y., Kim, M. S., Cao, Y. (2016). Versatile strategy for controlling the specificity and activity of engineered T cells. Proc Natl Acad Sci USA, 113(4), E450-458). The cognate ligand is FITC-poly(ethylene glycol (PEG)-DSPE (“DSPE-PEG-FITC”). FIG. 1B provides a schematic showing stimulation of CAR T cells by antigen presenting cells coated with the corresponding amphiphile vaccine.

To generate the DSPE-PEG-FITC vaccine, PE (phosphoethanolamine) lipid (e.g., DSPE) was dissolved in 500 μL CHCl₃ and 500 μL DMF, 3 eq of triethylamine and 1.2 eq of fluorescein-PEG2000-NHS (Creative PEG Works Inc.) was added and the reaction mixture were agitated overnight. The amphiphilic fluorescein PEG amphiphiles were purified by reverse phase HPLC using a C4 column (BioBasic-4, 200 mm×4.6 mm, Thermo Scientific), 100 mM triethylamine-acetic acid buffer (TEAA, pH 7.5)-methanol (0-30 min, 10-100%) as an eluent. The final products were dissolved in H₂O and quantified by UV-Vis spectroscopy (fluorescein, extinction coefficient 70,000 M⁻¹ cm⁻¹ at 490 nm, pH 9) and characterized by MALDI-TOF mass spectrometry. To generate the DSPE-PEG-peptide/protein ligand, N-terminal cysteine-modified peptides or protein ligand were dissolved in DMF and mixed with 2 equivalents maleimide-PEG2000-DSPE (Laysan Bio, Inc.), and the mixture was agitated at 25° C. for 24 hours. Bioconjugations were judged to be essentially complete by HPLC analysis. Peptide amphiphiles were characterized by MALDI-TOF mass spectrometry. The peptide conjugates were then diluted in 10×ddH₂O and lyophilized into powder, redissolved in H₂O and stored at −80° C.

### Example 2: In Vitro Activation of Anti-FITC CAR-T Cells by DSPE-PEG-FITC Coated Cells

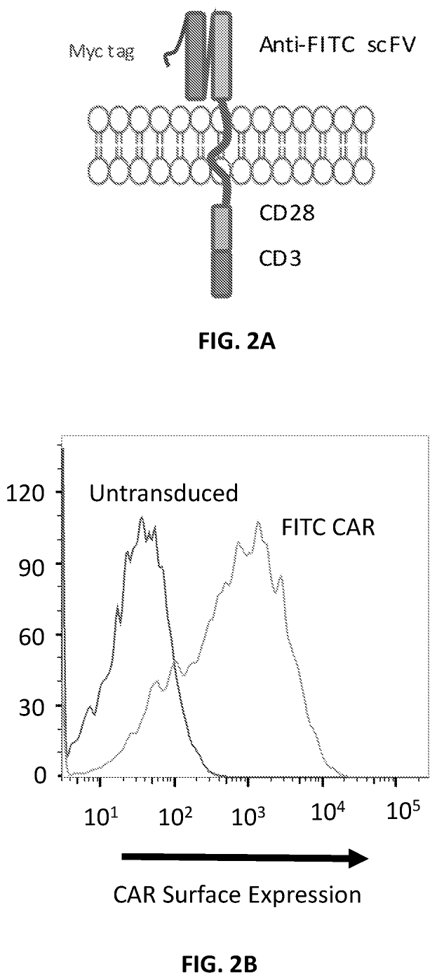

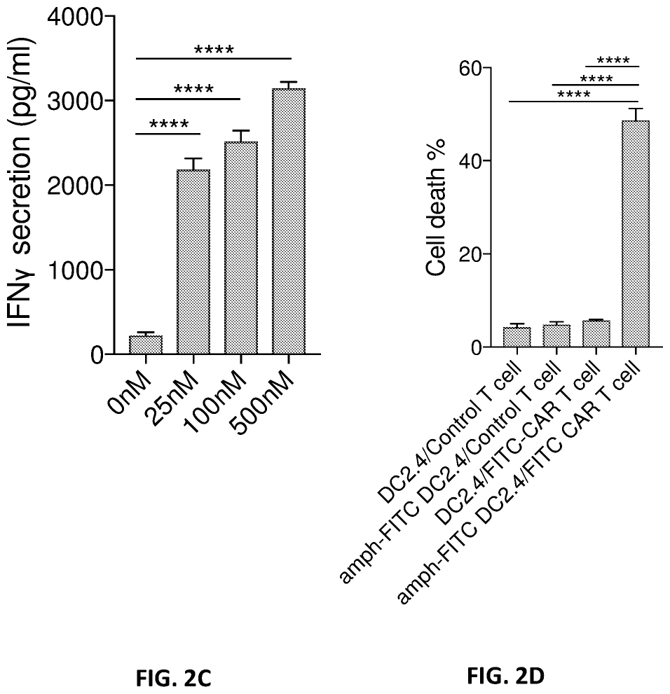

To determine the effect of an amphiphilic ligand conjugate on chimeric antigen receptor (CAR) T cells, in vitro stimulation of CAR-T cells was assessed after co-culture with antigen presenting cells (APCs) providing the amphiphilic ligand conjugate. Specifically, model CAR-T cells expressing anti-FITC CARs were generated by retroviral transduction of a DNA vector comprising an anti-FITC (fluorescein) scFV (4m5.3) coding region fused in-frame to a Myc epitope tag coding region and to a CAR coding region comprising a CD8 transmembrane domain, a CD28 signaling domain, and a CD3z signaling domain into primary mouse T cells. The domain structure and orientation of the Myc-tagged anti-FITC CAR is depicted in FIG. 2A. Surface expression of the Myc-tagged anti-FITC CAR in primary mouse T cells was quantified by incubating the transduced cells with a fluorescently-labeled anti-Myc antibody and quantifying the fluorescent cells by flow cytometry (FIG. 2B).

Next, model target cells, K562 cells, were tested for efficient membrane insertion of an amphiphilic ligand conjugate comprising a lipophilic moiety (i.e., DSPE) covalently linked to FITC via a PEG-2000 linker. At low doses (i.e., 25 nM) of DSPE-PEG-FITC, increasing serum concentration almost completely abolished surface insertion. However, at high doses (500 nM), DSPE-PEG-FITC retained a high level of cell surface decoration (data not shown).

To mimic antigen presenting cells in lymph nodes, dendritic cells (DC2.4) were decorated with increasing concentrations of DSPE-PEG-FITC, and then co-cultured with anti-FITC CAR T-cells for 0 h, 48 h, and 96 h. The ability of FITC-decorated DC2.4 cells to stimulate anti-FITC CAR T-cells was monitored by IFNγ secretion by CAR-T cells. Although most of the FITC molecules appeared to be internalized within 24 hours, strong induction of IFNγ by CAR-T cells was observed at 0 and 48 hours, then declined at 96 hours (data not shown), and dose-dependent activation was observed (FIG. 2C). Further, when FITC-decorated DC2.4 cells were co-cultured with FITC-CAR-T cells for 6 hours at an effector to target (E:T) ratio of 10:1, the DC2.4 cells were killed when FITC-CAR-T cells were administered with DSPE-PEG-FITC (FIG. 2D). In addition, as previously reported (Ma et al., 2016), co-culturing FITC-CAR T cells with CD19+ target cells in the presence of FITC-conjugated anti-CD19 antibody, but not a control antibody, resulted in potent CAR-T activation as determined by IFNγ secretion (data not shown). Overall, These results indicate that amphiphilic ligand conjugates are capable of activating CAR-T cells.

### Example 3: DSPE-PEG-FITC Trafficking to Lymph Node (LN), Retention and Uptake by APCs

Based on the results of Example 2, it was next determined whether the amphiphilic ligand conjugate DSPE-PEG-FITC could coat antigen presenting cells in lymph nodes (LN) to prime FITC-CAR-T cells in vivo. To assess DSPE-PEG-FITC trafficking to the lymph node and retention and uptake by APCs, C57BL/6 mice received varying doses of DSPE-PEG-FITC. Specifically, inguinal LN, auxiliary LN and lilac LN were harvested 24 hours after administration of 2 nmol, 5 nmol, or 10 nmol doses of DSPE-PEG-FITC was into the tail-veil of the mice. Free FITC was used as control. Mice were sacrificed and LNs were removed at different time point for IVIS imaging (excitation 465 nm, emission 520 nm) to monitor LN retention of FITC signal. The most efficient draining was into inguinal LN, followed by auxiliary LN (data not shown). At the high dose, DSPE-PEG-FITC was also observed to drain into the iliac LN.

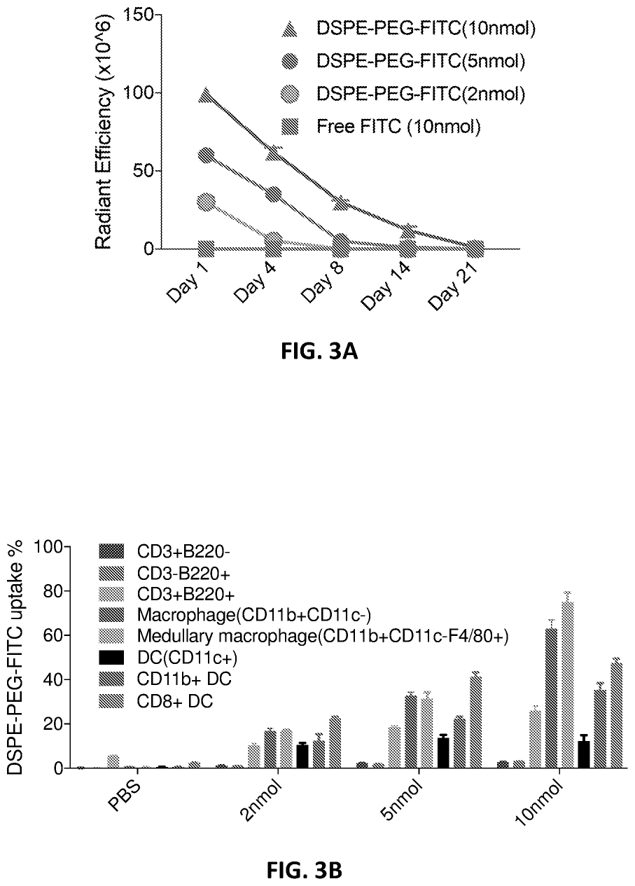

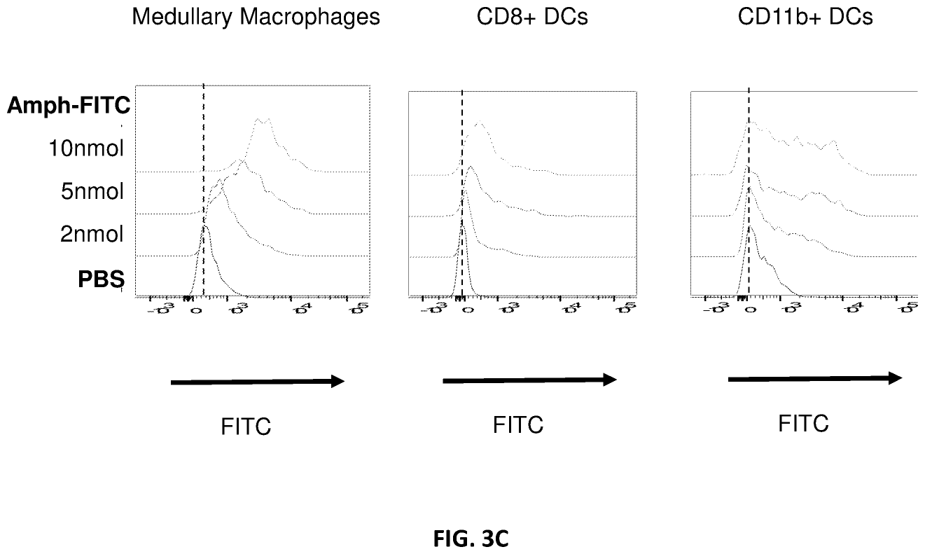

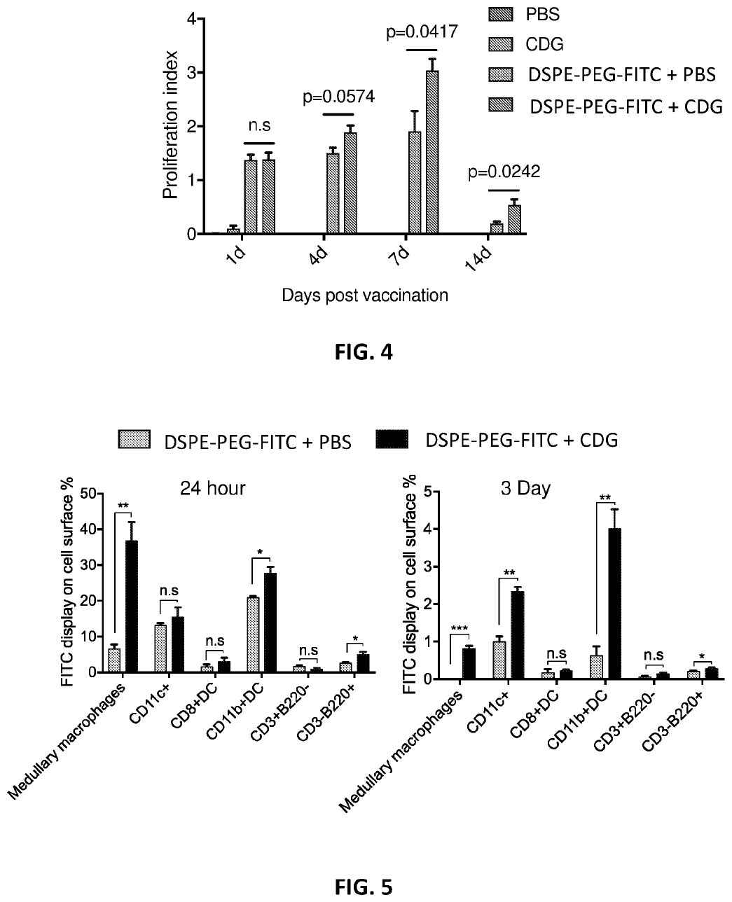

While FITC signal was almost lost at the lowest dose (2 nmol) after 4 days, the signal was retained for more than 21 days at high dose (10 nmol) of DSPE-PEG-FITC (FIG. 3A). Free FITC signal was lost in 24 hours (FIG. 3A). Flow cytometry analysis of LN cells revealed substantial uptake of DSPE-PEG-FITC in CD8+ and CD11b+ dendritic cells (DC), as well as macrophages, but minimal accumulating in T cells or B cells (FIGS. 3B and 3C). Confocal imaging of LNs showed that DSPE-PEG-ITC initially accumulated in interfollicular regions after 1 day, but partitioned onto CD11c+ DCs in T cell areas over time, and sorted FITC+CD11c+ cells from these LNs stained brightly with an anti-FITC antibody (data not shown).

Overall, these results indicate the amphiphilic ligand conjugate is expressed on antigen presenting cells in the lymph nodes.

### Example 4: DSPE-PEG-FITC Retained in the LN Robustly Stimulates CAR T-Cell Proliferation

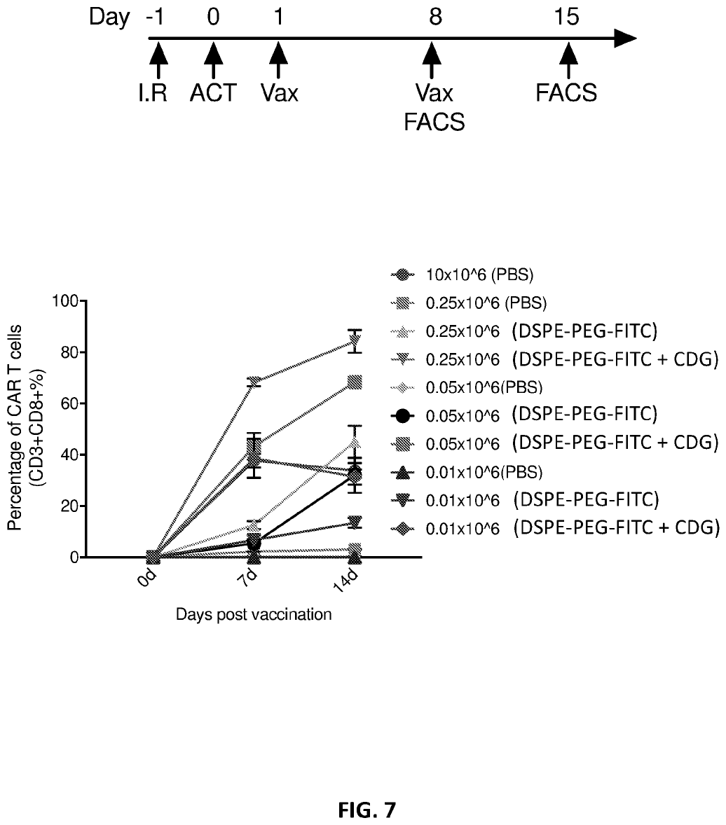

To assess whether DSPE-PEG-FITC accumulating on lymph node antigen presenting cells would lead to CAR T cell priming and how long this stimulatory effect would last for, at day 1, mice were administered PBS, c-di-GMP (25 ug), DSPE-PEG-FITC (10 nmol), or DSPE-PEG-FITC (10 nmol)+c-di-GMP (25 ug) into wildtype C57Bl/6 mice. After various time points, as indicated in the timeline in FIG. 7A, 2×10⁶ CTV-labeled CAR-T cells were transferred into each mouse via tail-vein injection. CAR-T cells were titrated to be a mixture of CAR+ and CAR-cells at 1:1 ratio. After another 48 hours, mice were sacrificed and LNs were removed for FACS analysis. As demonstrated in the representative results in FIG. 7B, up to 7 days post vaccination FITC-CAR-T were efficiently stimulated in lymph node 48 hours post adoptive transfer, and that co-administration of a strong T cell-promoting adjuvant, cyclic-di-GMP (CDG, a STING agonist) significantly extended DSPE-PEG-FITC stimulation up to 14 days (FIG. 7B). Minimal proliferation of CAR-T cells was observed in control mice receiving PBS or adjuvant alone. These results indicate the ability of an amphiphilic ligand conjugate to induce CAR-T cell proliferation in vivo.

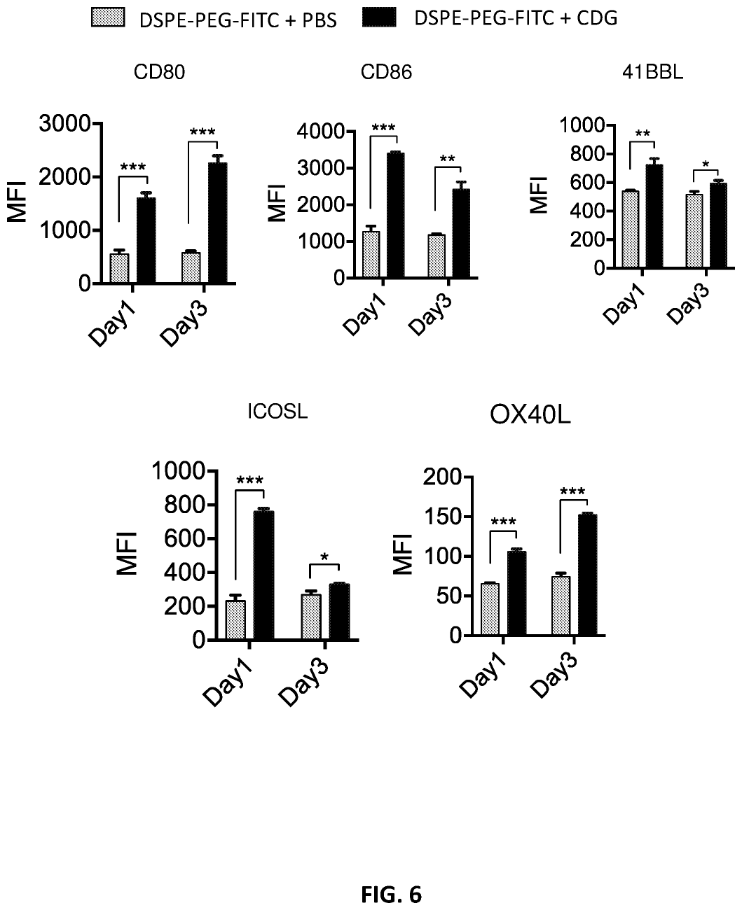

Further, CDG co-administration significantly increased duration and accessibility of DSPE-PEG-FITC on multiple APC cell surfaces, including macrophages and CD11c+CD11b+ DCs (FIG. 5). In addition, CDG co-administration increased expression level of several co-stimulatory molecules, i.e., CD80, CD86, 41BBL, ICOSL, and OX40L, relative to DSPE-PEG-FITC alone (FIG. 6). Expression was measured 24 hours and 3 days after vaccination.

### Example 5: Effect of DSPE-PEG-FITC on Long Term CAR-T Cell Expansion

To trace the effect of DSPE-PEG-FITC on the long-term in vivo expansion of CAR-T cells, a CD45.1/CD45.2 congenic transplantation model was utilized. Specifically, lymphodepleted CD45.2 recipient mice received various doses of CD45.1 donor FITC CAR-T cells (0.25×10⁶; 0.05×10⁶; 0.01×10⁶) at day 0.24 hours later, mice received PBS or vaccination with 10 nmol DSPE-PEG-FITC with or without 25 ug CDG. FIG. 7 provides a timeline of the experiment. The percentage of circulating CAR-T cells was determined by FACS analysis of peripheral blood collected at 7 and 14 days post vaccination. CAR T cells were defined as CD3+CD8+/Myc tag+ population.

A dramatic longitudinal CD45.1 CAR-T expansion was observed after vaccination with DSPE-PEG-FITC, alone or in combination with CDG. Specifically, the 0.25×10⁶ group took up >70%, and the 0.05×10⁶ group took up >50% of peripheral CD8+ T cells 7 days after the first vaccination, which was significantly more than mice transferred with 10×10⁶ ex vivo expanded CAR-T cells (FIG. 7). With a second boot, the 0.01×10⁶ group also reached 50% by day 14.

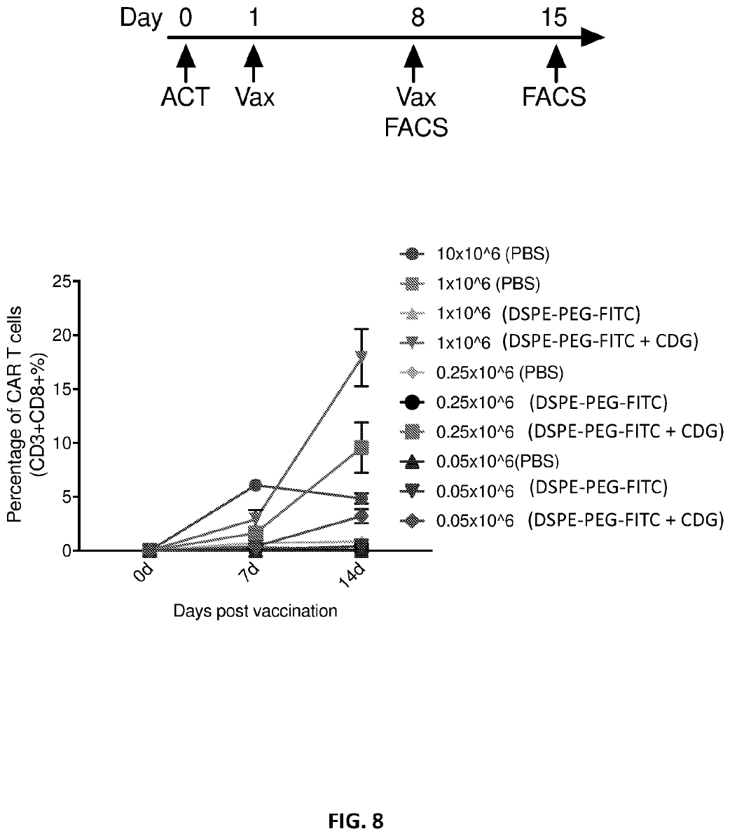

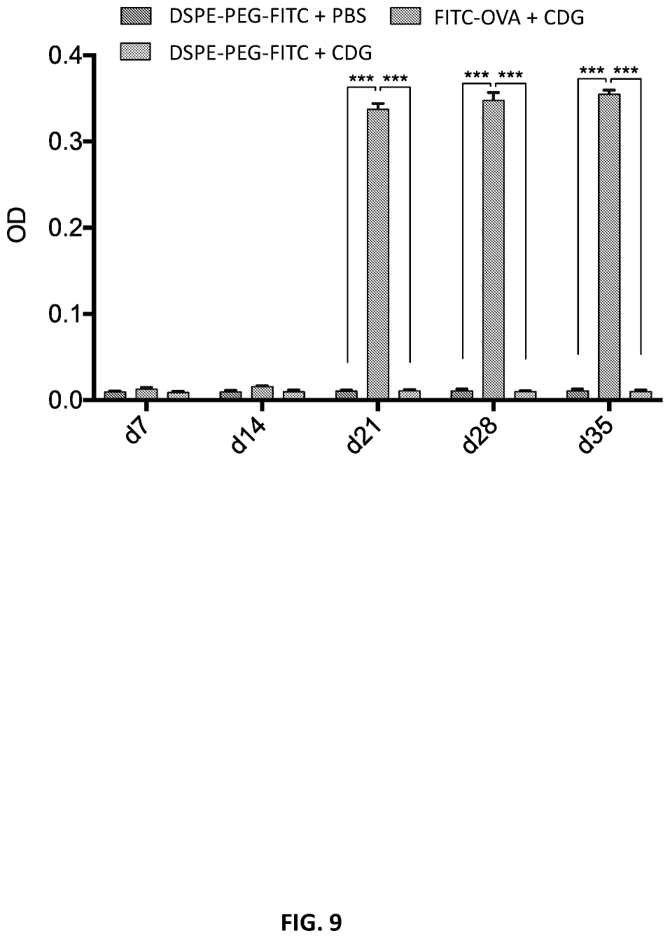

Further, the efficacy of DSPE-PEG-FITC was assessed in lymphreplete mice. Lymphodepleting regimens enhance the efficacy of adoptive cell therapy, but are associated with serious toxicities. Given the potent CAR-T boosting by DSPE-PEG-FITC in lymphodepleted setting, it was next considered whether DSPE-PEF-FITC could expand CAR-T cells to a considerable level in lymphreplete mice. Specifically, multiple doses of CD45.1 FITC CAR-T cells were transferred into lymphreplete CD45.2 recipient mice, followed by the same vaccination scheme and subsequent analysis described above, shown in FIG. 8. The results are also shown in FIG. 8, which indicate control mice that received 10×10⁶ CAR-T only had ˜5% circulating CD8+T cell population, while mice that received 0.25×10⁶ CAR-T plus DSPE-PEG-FITC reached ˜10% by day 14, and ˜20% was achieved in the 1×10⁶ CART-T group. One concern was that repeated vaccination may elicit antibody against FITC when conjugated to DSPE-PEG, thus blocking its stimulation to CAR in lymph nodes. However, no antibody response was observed when FITC was conjugated to DSPE-PEG or to the carrier protein OVA (FIG. 9), as the DSPE-PEG provided no source of T cell help.

Overall, these results indicated the DSPE-PEG-FITC vaccine in combination with an adjuvant (i.e., CDG) acted as a potent CAR-T booster vaccine in vivo.

### Example 6: Efficacy of an Amphiphile Vaccine Having a Tumor-Specific Antigen

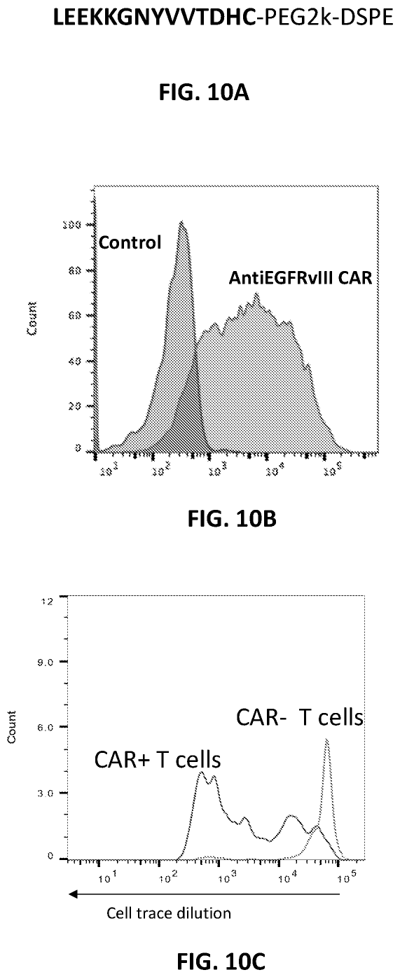

Next it was evaluated whether the same booster vaccine concept described in the Examples supra could be used for a bona fide tumor antigen-specific CAR. Specifically, the murine EGFRvIII-specific CAR 139scFv was utilized, which recognizes a short linear epitope derived from EGFRvIII (Sampson, et al. (2014). EGFRvIII mCAR-modified T-cell therapy cures mice with established intracerebral glioma and generates host immunity against tumor-antigen loss. Clin Cancer Res, 20(4), 972-984). Murine T cells were transduced with this CAR, and an amphiphile-EGFRvIII peptide vaccine molecule was synthesized by the following method: c-terminus cysteine-modified EGFRvIII peptide dissolved in dimethylformamide(DMSO) was mixed with 2.5 equivalents of 1,2-distearoyl-sn-glycero-3-phosphoethanolamine-N-[maleimide(polyethylene glycol)-2000] (DSPE-PEG2k) and 1 equivalent of tris(2-carboxyethyl)phosphine hydrochloride and a catalytic amount of triethylamine. The mixture was agitated at room temperature for 24 hours and subsequently purified by HPLC and dissolve in H2O. A schematic of the DSPE-PEG-EGFRvIII amphiphile vaccine is shown in FIG. 10A, and FIG. 10B shows expression of anti-EGFRvIII CAR on T cells.

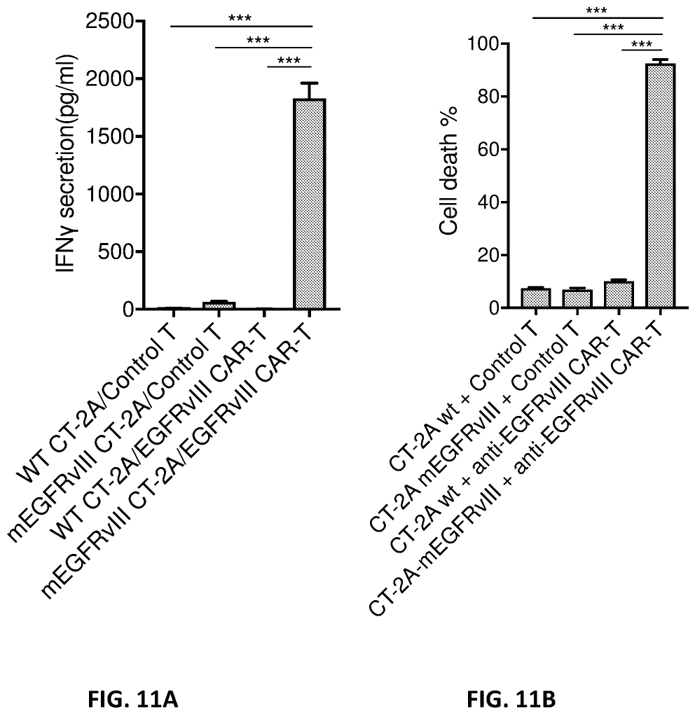

Similar to DSPE-PEG-FITC, DSPE-PEG-EGFRvIII inserted in cell membranes in vitro, and DSPE-PEG-EGFRvIII-coated cells stimulated EGFRvIII-CAR-T cells (data not shown). Further, immunization of mice with 10 ug of DSPE-PEG-EGFRvIII and adjuvant (25 ug of cyclic-di-GMP) 24 hours after intravenous injection of 2×10⁶ cell trace violet (CTV) labeled EGFRvIII-CAR-T cells triggered extensive CAR-T cell proliferation in draining inguinal lymph node in vivo after 48 hours (FIG. 10C). To test the therapeutic impact of vaccine boosting, murine CT-2A glioma cells were transduced with EGFRvIII and co-cultured with EGFRvIII-CAR-T cells. The CAR-T cells secreted IFNγ in the presence of the EGFRvIII expressing CT-2A glioma cells (FIG. 11A). Further, co-culturing CT-2A glioma cells expressing wildtype EGFR or EGFRvIII with EGFRvIII-CAR-T at 1:10 ratio for 6 hours in vitro resulted in efficiently killing of EGFRvIII-expressing but not wildtype EGFR expressing CT-2A glioma cells by EGFRvIII-CAR-T cells (FIG. 11B).

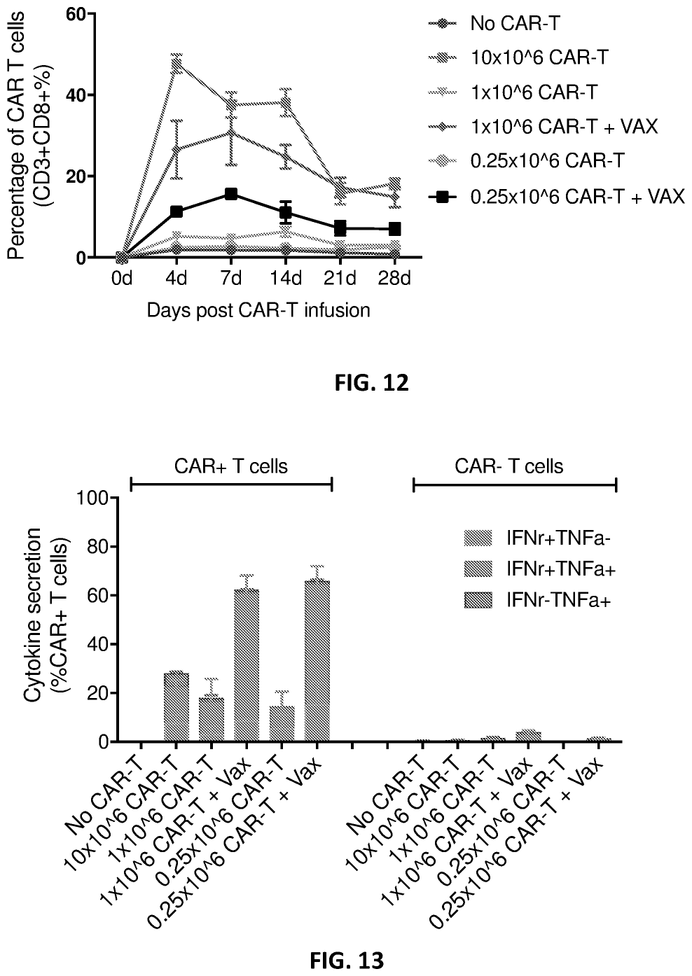

To further investigate the efficacy of the DSPE-PEG-EGFRvIII amphiphile vaccine, an in vivo model was utilized. Specifically, wildtype CD45.2 C57Bl/6 mice were implanted with 4×10⁶ EGFRvIII expressing CT-2A cells. At day 7, the CT-2A-mEGFRvIII tumor-bearing mice received sublethal irradiation and subsequent infusion of different doses of EGFRvIII CAR-T cells produced from CD45.1 mice, followed with or without 10 ug of DSPE-PEG-EGFRvIII plus 25 ug of CDG. In the group that received 10×10⁶ CAR-T cells, circulating CAR-T cells accounted for ˜40% of peripheral blood CD8+ T cells (FIG. 12). Mice that received lower cell number had minimal circulating CAR-T cells, yet dramatic EGFRvIII CAR-T expansion was achieved in groups that received DSPE-PEG-EGFRvIII plus CDG (FIG. 12).

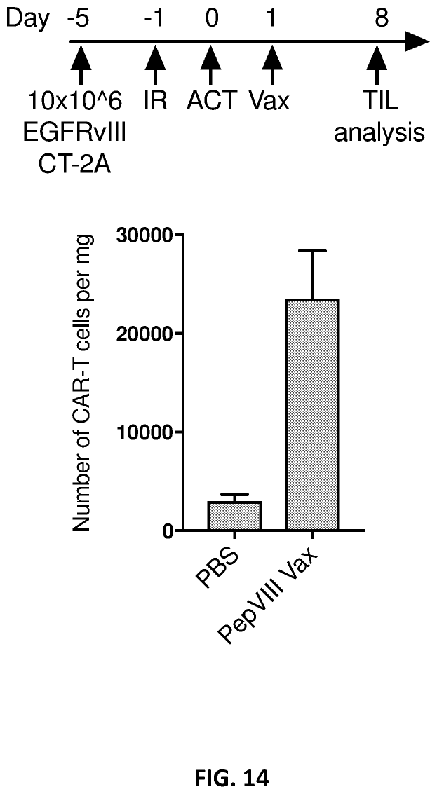

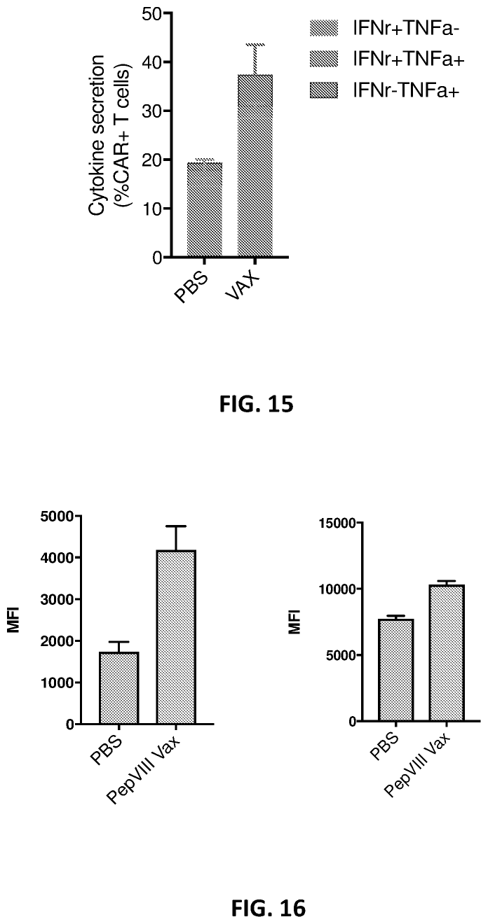

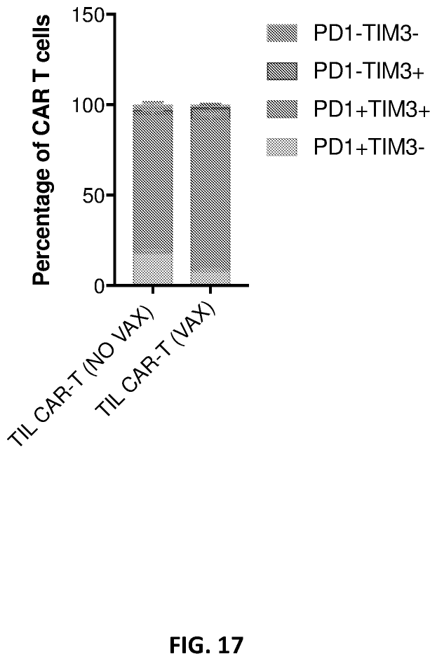

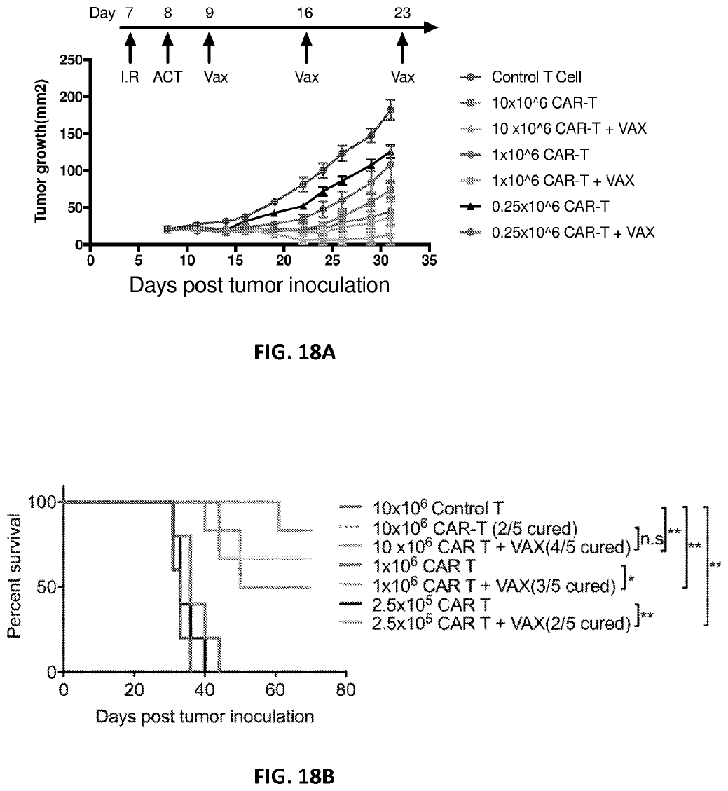

To assess the impact of the amphiphilic ligand conjugate on EGFRvIII CAR T function, intracellular cytokine staining (ICS) was performed by using peripheral blood collected 7 days after vaccination. Peripheral blood mononuclear cells (PBMCs) were mixed with EGFRvIII expressing CT-2A cells at 1:1 ratio in 96-well plate for 6 hours in the presence of 1× golgiplug. Cells were then surface stained, fixed and permeabilized, then further stained with anti-IFNγ and anti-TNFα antibodies to evaluate cytokine production of vaccine boosted or unboosted EGFRvIII CAR T cells in response target cells. DSPE-PEG-EGFRvIII boosted EGFRvIII CAR-T cells had significantly enhanced functionality, with the majority of circulating CAR-T responding to target tumor cells (FIG. 13). Moreover, significantly increased CAR-T infiltration into tumor in the DSPE-PEG-EGFRvIII+CDG boosted group was observed at day 7 after vaccination, as determined by FACS analyzing the number of CAR-T cells per mg of tumor (FIG. 14). In addition, tumor infiltrating CAR-T cells exhibited enhanced reactivity against tumor cells 7 days after vaccination. Specifically, FIG. 15 shows the level of cytokine secretion by tumor infiltrating CAR-T cells was enhanced in the presence of DSPE-PEG-EGFRvIII+CDG relative to PBS, whereas FIG. 16 shows the level of granzyme B, an indicator of cytotoxicity, increased in tumor infiltrating CAR-T cells, and Ki67, an indicator of proliferation, was also increased. Interestingly, this enhanced reactivity occurred despite surface expression of PD1 and TIM3 (FIG. 17) Animals that received both CAR-T and DSPE-PEG-EGFRvIII+CDG had significantly delayed tumor growth (FIG. 18A) and prolonged survival (FIG. 18B). Notably, similar to DSPE-PEG-FITC vaccinated mice, no antibody response was elicited against EGFRvIII after four rounds of weekly vaccination, and only slight weight loss was observed following each vaccination, which indicated toxicity is at a manageable level (data not shown).

### Example 7: Design and Efficacy of a Bispecific CAR T Cells Vaccinated with DSPE-PEG-FITC

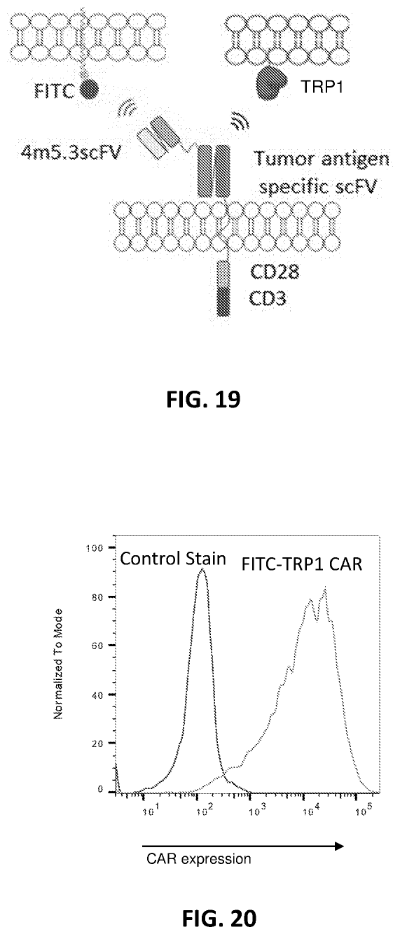

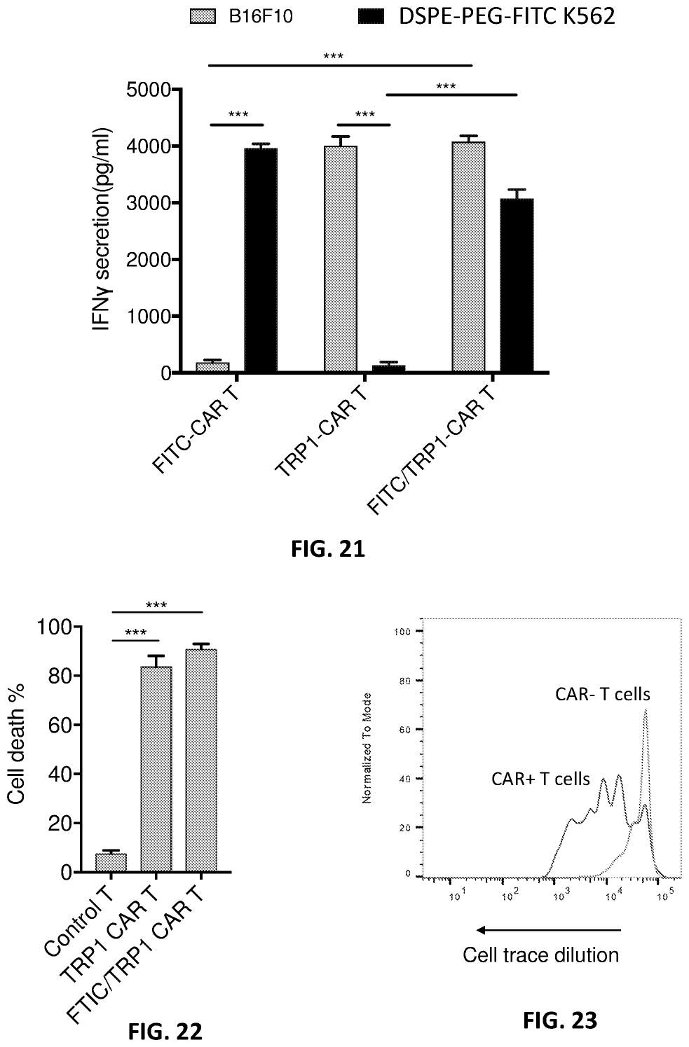

Use of a surrogate peptide ligand for CAR T cells is effective, but some CARs recognize three-dimensional structural epitopes (De Oliveira, et al. (2013). A CD19/Fc fusion protein for detection of anti-CD19 chimeric antigen receptors. J Transl Med, 11, 23. doi:10.1186/1479-5876-11-23) for which it may be difficult or impossible to identify a simple surrogate ligand. To eliminate such limitations and provide a means to boost any CAR regardless of the nature of its binding domain or its specificity, a tandem scFv-based bispecific CAR was designed. Specifically, an anti-FITC scFV 4m5.3 was appended to the N-terminal extracellular domain of a tumor-targeting CAR via a (G45)₄ peptide linker immediately after the N-terminal signal peptide (FIG. 19). To evaluate the feasibility of this approach, a bispecific murine CAR targeting both FITC and the melanoma-associated antigen TRP1, which expressed well in primary mouse T cells was utilized. FIG. 20 shows expression of the bispecific CAR on T cells. To confirm the reaction specificity of this bispecific CAR, FITC/TRP1-CAR T cells were co-cultured with either DSPE-PEG-FITC-coated target cells or TRP1-expressing B16F10 cells at 10:1 effector:target ratio for 6 hours in vitro, FITC/TRP1-CAR T responded to both antigens specifically and potently as indicated by IFNγ secretion (FIG. 21). Further, FITC/TRP1-CAR T cells killed TRP1+ target cells equivalently to mono-specific TRP1-CAR T cells, as determined by co-culturing the cells for 6 hours at an effector to target (E:T) ratio of 10:1 (FIG. 22). In vivo, DSPE-PEG-FITC vaccination robustly stimulated FITC/TRP1 bispecific CAR-T proliferation, as determined by cell trace violet trafficking 48 hours after vaccination (FIG. 23).

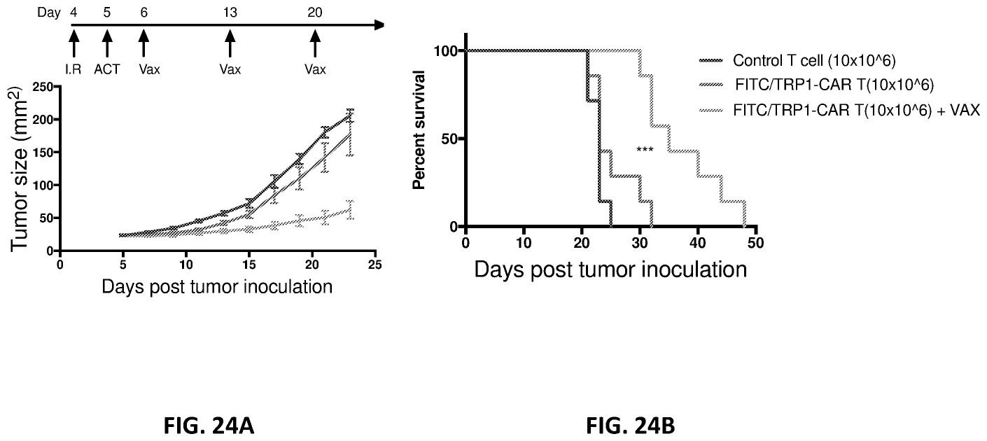

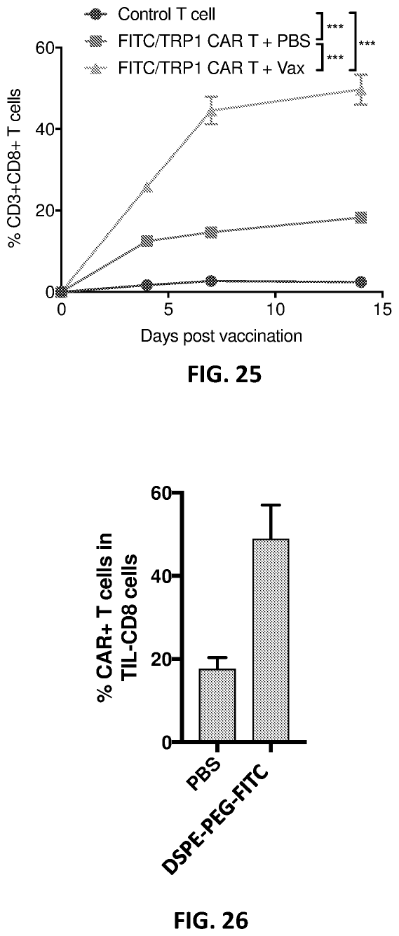

To assess the therapeutic potential of bispecific CAR-T with DSPE-PEG-FITC plus CDG, wildtype C57Bl/6 mice were implanted with 5×10⁵ B16F10 tumor cells. 5 days later, tumor bearing animals received lymphodepeletion treatment with 500 cGy irradiation followed by intravenous injection of 10×10⁶ FITC/TRP1 CAR-T cells the next day. CAR-T cells alone had a slight impact on tumor growth compared to control T cells, whereas mice that received both CAR-T and vaccine (10 nmol DSPE-PEG-FITC+25 ug CDG) exhibited dramatically delayed tumor growth (FIG. 24A) and significantly prolonged survival (FIG. 24B). This was consistent with increased circulating CAR-T levels (FIG. 25) and enhanced tumor infiltration (FIG. 26). Body weight loss was also under control in animal groups that received vaccination (data not shown).

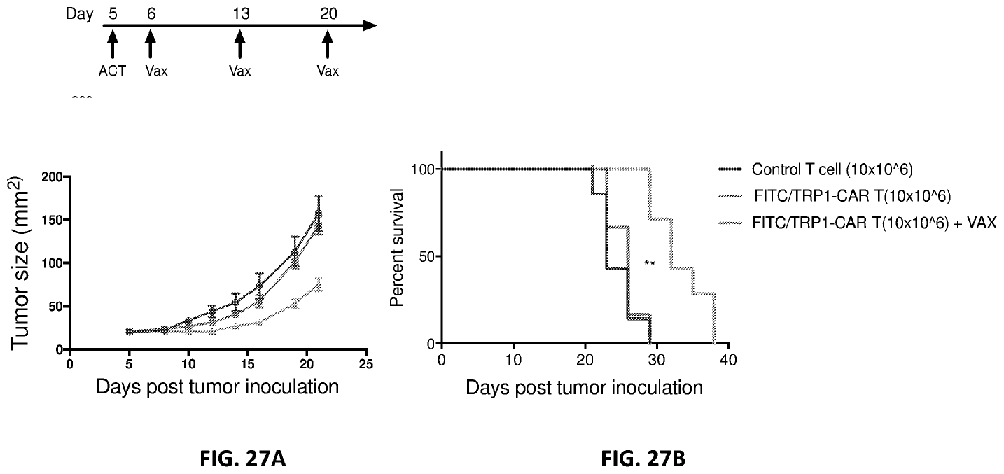

Motivated by the potent CAR-T expansion and functional enhancement with DSPE-PEG-FITC+CDG boosting, the therapeutic efficacy of CAR-T plus DSPE-PEG-FITC+CDG in tumor bearing mice without lymphodepletion preconditioning was evaluated. To facilitate in vivo tracking, CD45.1 FITC/TRP1 CAR-T cells were transferred into CD45.2 recipient mice with B16F10 melanoma, following a similar vaccination scheme. Mice that received CAR-T alone had almost indistinguishable tumor growth as those that received control T cells, whereas the combination treatment with both CAR-T and DSPE-PEG-FITC+CDG vaccination significantly delayed tumor growth and increased animal survival (FIGS. 27A and 27B) with minimal impact on animal weight (data not shown).

Although DSPE-PEG-FITC preferentially traffics to and accumulates in lymph nodes, and incorporate into residing APCs in lymph nodes, a small percentage of amphiphile may leak into peripheral blood and insert into bystander cells making them de novo CAR-T targets. To analyze such unintended toxicity caused by amphiphiles escaped from lymphatic drainage, DSPE-PEG-FITC was intravenously injected into NSG mice, which have severely defective lymphatics, to stimulate FITC CAR-T cells. Nonetheless, there was negligible stimulation of FITC CAR-T proliferation (data not shown).

Overall, these results indicated CAR-T cell therapy is effective in solid tumors with the use of an amphiphile vaccine.

### EQUIVALENTS

Those skilled in the art will recognize or be able to ascertain, using no more than routine experimentation, many equivalents of the specific embodiments described herein described herein. Such equivalents are intended to be encompassed by the following claims.

What is claimed is:

1. An amphiphilic ligand conjugate comprising:
a chimeric antigen receptor (CAR) ligand; and
a lipid operably linked to the CAR ligand.

2. The amphiphilic ligand conjugate of claim 1, wherein the lipid inserts in a cell membrane under physiological conditions, binds albumin under physiological conditions, or both.

3. The amphiphilic ligand conjugate of claim 1 or claim 2, wherein the lipid is diacyl lipid.

4. The amphiphilic ligand conjugate of claim 3, wherein the diacyl lipid comprises acyl chains comprising 12-30 hydrocarbon units, 14-25 hydrocarbon units, 16-20 hydrocarbon units, or 12, 13, 14, 15, 16, 17, 18, 19, 20, 21, 22, 23, 24, 25, 26, 27, 28, 29 or 30 hydrocarbon units.

5. The amphiphilic ligand conjugate of any one of claims 1-4, wherein the CAR ligand is operably linked to the lipid via a linker.

6. The amphiphilic ligand conjugate of claim 5, wherein the linker is selected from the group consisting of hydrophilic polymers, a string of hydrophilic amino acids, polysaccharides, or a combination thereof.

7. The amphiphilic ligand conjugate of claim 5, wherein the linker comprises “N” consecutive polyethylene glycol units, wherein N is between 25-50.

8. An amphiphilic ligand conjugate comprising, a CAR ligand operably linked to a diacyl lipid via a linker, wherein the diacyl lipid comprises acyl chains comprising 12-30 hydrocarbon units, and wherein the linker comprises “N” consecutive polyethylene glycol units, wherein N is between 25-50.

9. The amphiphilic ligand conjugate of any one of claims 1-8, wherein the CAR ligand is a tag.

10. The amphiphilic ligand conjugate of claim 9, wherein the tag is selected from the group consisting of fluorescein isothiocyanate (FITC), streptavidin, biotin, dinitrophenol, peridinin chlorophyll protein complex, green fluorescent protein, phycoerythrin (PE), horse radish peroxidase, palmitoylation, nitrosylation, alkalanine phosphatase, glucose oxidase, and maltose binding protein.

11. The amphiphilic ligand conjugate of any one of claims 1-8, wherein the CAR ligand is a tumor-associated antigen, or a fragment thereof.

12. An amphiphilic ligand conjugate comprising, a lipid operably linked to fluorescein isothiocyanate (FITC) via a polyethylene glycol moiety.

13. The amphiphilic ligand conjugate of any one of claims 8-12, wherein the lipid is 1,2-distearoyl-sn-glycero-3-phosphoethanolamine (DSPE) and wherein the polyethylene glycol moiety is PEG-2000.

14. The amphiphilic ligand conjugate of any one of claims 1-13, wherein the CAR ligand binds to a CAR, and wherein the CAR comprises a co-stimulation domain.

15. The amphiphilic ligand conjugate of claim 14, wherein the CAR comprises a bispecific binding domain.

16. The amphiphilic ligand conjugate of claim 15, wherein the bispecific binding domain comprises a tag binding domain and a tumor-associated antigen binding domain or comprises a first tumor-associated antigen binding domain and a second tumor associated antigen binding domain.

17. The amphiphilic ligand conjugate of claim 16, wherein the bispecific binding domain comprises a tag binding domain and a tumor-associated antigen binding domain, and wherein the CAR ligand is a tag.

18. The amphiphilic ligand conjugate of claim 15, wherein the bispecific binding domain comprises a first tumor-associated antigen binding domain and a second tumor-associated antigen binding domain, and wherein the CAR ligand is the first or second tumor-associated antigen, or fragment thereof.

19. The amphiphilic ligand conjugate of claim 14, wherein the CAR comprises a tag binding domain, and wherein the CAR ligand is a tag.

20. The amphiphilic ligand conjugate of claim 14, wherein the CAR comprises a tumor-associated antigen binding domain, and wherein the CAR ligand is a tumor-associated antigen or a fragment thereof.

21. A composition comprising the amphiphilic ligand conjugate of any one of claims 1-19, and a pharmaceutically acceptable carrier.

22. An immunogenic composition comprising the composition of claim 21, and an adjuvant.

23. The immunogenic composition of claim 22, wherein the adjuvant is an amphiphilic oligonucleotide conjugate comprising an immunostimulatory oligonucleotide conjugated to a lipid, with or without a linker, and optionally a polar compound.

24. The immunogenic composition of claim 23, wherein the immunostimulatory oligonucleotide binds a pattern recognition receptor.

25. The immunogenic composition of claim 24, wherein the immunostimulatory oligonucleotide comprises CpG.

26. The immunogenic composition of claim 23, wherein the immunostimulatory oligonucleotide is a ligand for a toll-like receptor.

27. The immunogenic composition of any one of claims 23-26, wherein the linker is an oligonucleotide linker.

28. The immunogenic composition of claim 27, wherein the oligonucleotide linker comprises “N” consecutive guanines, wherein N is between 0-2.

29. The immunogenic composition of any one of claims 23-28, wherein the lipid is a diacyl lipid.

30. The immunogenic composition of claim 29, wherein the diacyl lipid comprises acyl chains comprising 12-30 hydrocarbon units.

31. The immunogenic composition of claim 22, wherein the adjuvant is a cyclic di-GMP (CDG).

32. A method of activating, expanding or increasing proliferation of CAR-T cells in a subject, comprising administering to the subject the amphiphilic ligand conjugate of any one of claims 1-20, the composition of claim 21, or the immunogenic composition of any one of claims 23-31.

33. The method of claim 32, wherein the proliferation of CAR(−) T cells is not increased in the subject.

34. A method of reducing or decreasing a size of a tumor or inhibiting a tumor growth in a subject in need thereof, comprising administering to the subject the amphiphilic ligand conjugate of any one of claims 1-20, the composition of claim 21, or the immunogenic composition of any one of claims 23-31, wherein the subject is receiving or has received CAR-T cell therapy.

35. A method of inducing an anti-tumor response in a subject with cancer, comprising administering to the subject the amphiphilic ligand conjugate of any one of claims 1-20, the composition of claim 21, or the immunogenic composition of any one of claims 23-31, wherein the subject is receiving or has received CAR-T cell therapy.

36. A method of stimulating an immune response to a target cell population or target tissue expressing an antigen in a subject, the method comprising administering to the subject CAR-T cells targeted to the antigen, and the amphiphilic ligand conjugate of any one of claims 1-20, the composition of claim 21, or the immunogenic composition of any one of claims 23-31.

37. The method of claim 36, wherein the immune response is a T-cell mediated immune response or an anti-tumor immune response.

38. The method of claim 36 or claim 37, wherein the target cell population or target tissue is tumor cells or tumor tissue.

39. A method of treating a subject having a disease, disorder or condition associated with expression or elevated expression of an antigen, comprising administering to the subject CAR-T cells targeted to the antigen, and the amphiphilic ligand conjugate of any one of claims 1-20, the composition of claim 21, or the immunogenic composition of any one of claims 23-31.

40. The method of any one of claims 32-34, wherein the subject is administered the amphiphilic ligand conjugate, the composition or the immunogenic composition prior to receiving CAR T cells.

41. The method of any one of claims 32-34, wherein the subject is administered the amphiphilic ligand conjugate, the composition or the immunogenic composition after receiving CAR-T cells.

42. The method of any one of claims 32-34, wherein the amphiphilic ligand conjugate, the composition or the immunogenic composition, and CAR-T cells are administered simultaneously.

43. The method of any one of claims 32-42, wherein the amphiphilic ligand conjugate is trafficked to the lymph nodes.

44. The method of any one of claims 32-42, wherein the amphiphilic ligand conjugate is trafficked to the inguinal lymph node and auxiliary lymph node.

45. The method of any one of claims 32-44, wherein the amphiphilic ligand conjugate is inserted into the membrane of antigen presenting cells upon trafficking to the lymph nodes.

46. The method of claim 45, wherein the antigen presenting cells are medullary macrophages, CD8+ dendritic cells, and/or CD11b+ dendritic cells.

47. The method of any one of claims 32-46, wherein the CAR ligand is retained in the lymph nodes for at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, or at least 25 days.

48. The method of any one of claims 32-47, wherein the CAR ligand is tag, wherein the CAR comprises a tag binding domain, and wherein the method further comprises administering a formulation of tagged proteins, and wherein the tag binding domain binds the tagged proteins.

49. The method of claim 48, wherein the protein of the tagged protein is an antibody or an antigen-binding fragment thereof.

50. The method of claim 48 or claim 49, wherein the tag binding domain is an antibody or an antigen-binding fragment thereof.

51. The method of any one of claims 48-50, wherein the formulation of tagged proteins is administered to the subject prior to administration of the CAR-T cells and amphiphilic ligand conjugate, composition, or immunogenic composition.

52. The method of any one of claims 48-50, wherein the formulation of tagged proteins is administered to the subject concurrently with administration of the CAR-T cells and amphiphilic ligand conjugate, composition, or immunogenic composition.

53. The method of any one of claims 48-50, wherein the formulation of tagged proteins is administered to the subject after administration of the CAR-T cells and amphiphilic ligand conjugate, composition, or immunogenic composition.

54. The method of any one of claims 51-53, wherein the CAR-T cells are administered prior to administration of the amphiphilic ligand conjugate, composition, or immunogenic composition.

55. The method of any one of claims 51-53, wherein the CAR-T cells are administered after administration of the amphiphilic ligand conjugate, composition, or immunogenic composition.

56. The method of any one of claims 51-53, wherein the CAR-T cells are administered concurrently with administration of the amphiphilic ligand conjugate, composition, or immunogenic composition.

57. The method of any one of claims 32-34 and 49-56, wherein the subject has cancer.

58. The method of any one of claims 32-57, wherein the subject is a human.

59. A kit comprising a container comprising a composition the amphiphilic ligand conjugate of any one of claims 1-20, an optional pharmaceutically acceptable carrier, and a package insert comprising instructions for administration of the composition for treating or delaying progression of cancer in an individual receiving CAR-T cell therapy.

60. A kit comprising a medicament comprising a composition comprising the amphiphilic ligand conjugate of any one of claims 1-20, an optional pharmaceutically acceptable carrier, and a package insert comprising instructions for administration of the medicament alone or in combination with a composition comprising an adjuvant and an optional pharmaceutically acceptable carrier, for treating or delaying progression of cancer in an individual receiving CAR-T cell therapy.

61. A kit comprising a container comprising a composition comprising the amphiphilic ligand conjugate of any one of claims 1-20, an optional pharmaceutically acceptable carrier, and a package insert comprising instructions for administration of composition vaccine for activating, expanding or increasing proliferation of CAR-T cells in an individual receiving CAR-T cell therapy.

62. A kit comprising a medicament comprising a composition comprising the amphiphilic ligand conjugate of any one of claims 1-20, an optional pharmaceutically acceptable carrier, and a package insert comprising instructions for administration of the medicament alone or in combination with a composition comprising an adjuvant and an optional pharmaceutically acceptable carrier, for activating, expanding or increasing proliferation of CAR-T cells in an individual receiving CAR-T cell therapy.

63. The kit of claim 59 or claim 61, further comprising an adjuvant and instructions for administration of the adjuvant for treating or delaying progression of cancer in an individual receiving CAR-T cell therapy.

64. The kit of any one of claims 60, 62 and 63, wherein the adjuvant is an amphiphilic oligonucleotide conjugate comprising an immunostimulatory oligonucleotide conjugated to a lipid with or without a linker, and optionally a polar compound.

65. Use of the amphiphilic ligand conjugate of any one of claims 1-20, the composition of claim 21, or the immunogenic composition of any one of claims 23-31, for activating, expanding or increasing proliferation of CAR-T cells in an individual receiving CAR-T cell therapy.

66. Use of the amphiphilic ligand conjugate of any one of claims 1-20, the composition of claim 21, or the immunogenic composition of any one of claims 23-31, for treating or delaying progression of cancer in an individual.

67. Use of the amphiphilic ligand conjugate of any one of claims 1-20, the composition of claim 21, or the immunogenic composition of any one of claims 23-31, in the manufacture of a medicament for treating or delaying progression of cancer in an individual.

68. The method of any one of claims 32-58, comprising administering the amphiphilic ligand conjugate, the composition or the immunogenic composition parenterally at a non-tumor draining lymph node, parenterally at a tumor-draining lymph node, or intratumorally.

69. The method of claim 36, wherein the target cell population or target tissue is a population of cells or tissue infected with a virus.

70. The method of claim 69, wherein the virus is human immunodeficiency virus (HIV).

71. The method of claim 69 or claim 70, wherein the immune response is a T-cell mediated immune response.

72. The method of claim 39, wherein the antigen is a viral antigen or caner antigen.

73. A kit comprising a medicament comprising a composition comprising the amphiphilic ligand conjugate of any one of claims 1-20, an optional pharmaceutically acceptable carrier, and a package insert comprising instructions for administration of the composition for treating or delaying progression of a viral infection in an individual receiving CAR-T cell therapy.

74. The kit of claim 73, further comprising a formulation of tagged proteins and instructions for administration of the formulation of tagged proteins, wherein the CAR comprises a tag binding domain that binds the tagged proteins.

75. The kit of claim 73 or claim 74, further comprising an adjuvant and instructions for administration of the adjuvant for treating or delaying progression of a viral infection in an individual receiving CAR-T cell therapy.

76. The kit of claim 75, wherein the adjuvant is an amphiphilic oligonucleotide conjugate comprising an immunostimulatory oligonucleotide conjugated to a lipid with or without a linker, and optionally a polar compound.


SEQUENCE LISTING (2 sequences)

SEQ ID NO: 1 - protein, 14 aa, Artificial Sequence
  Synthetic EGFRvIII peptide conjugated to DSPE-PEG
LEEKKGNYVVTDHC

SEQ ID NO: 2 - DNA, 25 bp, Artificial Sequence
  Synthetic lipophilic-CpG oligonucleotide
  misc_feature (1)..(5): at least one “g” is present and up to 4 “g” may be present or absent
  modified_base (1)..(1): Lipophilic compound
gggggtccatgacgttcctgacgtt